SEQUENCE LISTING (218 sequences)

SEQ ID NO: 1 - DNA, 24 bp, Artificial Sequence
  Description of Artificial Sequence Synthetic primer
ctgaaacatcttagtaagcagagg

SEQ ID NO: 2 - DNA, 21 bp, Artificial Sequence
  Description of Artificial Sequence Synthetic primer
gtgtctagtcctactcaggtg

SEQ ID NO: 3 - DNA, 41 bp, Artificial Sequence
  Description of Artificial Sequence Synthetic primer
cctacaaccccgagccttatcaagagattccctgtgtagcg

SEQ ID NO: 4 - DNA, 45 bp, Artificial Sequence
  Description of Artificial Sequence Synthetic primer
ggactcctagttgaacacatctggattctctcctttcgtctacgg

SEQ ID NO: 5 - DNA, 20 bp, Artificial Sequence
  Description of Artificial Sequence Synthetic primer
gctcggtttaggctattccc

SEQ ID NO: 6 - DNA, 25 bp, Artificial Sequence
  Description of Artificial Sequence Synthetic primer
aagatggatgatacagggtgatagt

SEQ ID NO: 7 - DNA, 42 bp, Artificial Sequence
  Description of Artificial Sequence Synthetic primer
atcctttatcctcaatcctacaaccgtagcggcgagcgaaag

SEQ ID NO: 8 - DNA, 18 bp, Artificial Sequence
  Description of Artificial Sequence Synthetic primer
agccttatcagctcggtt

SEQ ID NO: 9 - DNA, 16 bp, Artificial Sequence
  Description of Artificial Sequence Synthetic primer
gacacctgccgaactg

SEQ ID NO: 10 - DNA, 16 bp, Artificial Sequence
  Description of Artificial Sequence Synthetic primer
agccttggagagtggt

SEQ ID NO: 11 - DNA, 44 bp, Artificial Sequence
  Description of Artificial Sequence Synthetic primer
tcctgatcctttatcctcaatcctacgagattccctgtgtagcg

SEQ ID NO: 12 - DNA, 41 bp, Artificial Sequence
  Description of Artificial Sequence Synthetic primer
tcctagttgaacacatctggaaagaaggtgttgaggtcggt

SEQ ID NO: 13 - DNA, 20 bp, Artificial Sequence
  Description of Artificial Sequence Synthetic primer
ccgtagacgaaaggagagaa

SEQ ID NO: 14 - DNA, 19 bp, Artificial Sequence
  Description of Artificial Sequence Synthetic primer
cctgccgaactgaaacatc

SEQ ID NO: 15 - DNA, 18 bp, Artificial Sequence
  Description of Artificial Sequence Synthetic primer
ggtgttgaggtcggtctt

SEQ ID NO: 16 - DNA, 41 bp, Artificial Sequence; identical to SEQ ID NO: 3
  Description of Artificial Sequence Synthetic primer

SEQ ID NO: 17 - DNA, 46 bp, Artificial Sequence
  Description of Artificial Sequence Synthetic primer
ggatcaggactcctagttgaacacactttcgtctacgggactatca

SEQ ID NO: 18 - DNA, 23 bp, Artificial Sequence
  Description of Artificial Sequence Synthetic primer
ctggaaagatggatgatacaggg

SEQ ID NO: 19 - DNA, 24 bp, Artificial Sequence
  Description of Artificial Sequence Synthetic primer
gggttgtaggattgaggataaagg

SEQ ID NO: 20 - DNA, 24 bp, Artificial Sequence
  Description of Artificial Sequence Synthetic primer
ggttcactatcggtcattgactag

SEQ ID NO: 21 - DNA, 48 bp, Artificial Sequence
  Description of Artificial Sequence Synthetic primer
tttctctcctttcgtctacgggactatcaggactcctagttgaacaca

SEQ ID NO: 22 - DNA, 44 bp, Artificial Sequence
  Description of Artificial Sequence Synthetic primer
accgacctcaacacctgagtaggttagccttggagagtggtctc

SEQ ID NO: 23 - DNA, 25 bp, Artificial Sequence
  Description of Artificial Sequence Synthetic primer
caccctgtatcatccatctttccag

SEQ ID NO: 24 - DNA, 24 bp, Artificial Sequence
  Description of Artificial Sequence Synthetic primer
cgtgaaacctagtctgaatctggg

SEQ ID NO: 25 - DNA, 19 bp, Artificial Sequence
  Description of Artificial Sequence Synthetic primer
gtaggattgaggataaagg

SEQ ID NO: 26 - DNA, 18 bp, Artificial Sequence
  Description of Artificial Sequence Synthetic primer
cagtactggttcactatc

SEQ ID NO: 27 - DNA, 39 bp, Artificial Sequence
  Description of Artificial Sequence Synthetic primer
tctttctctcctttcgtctacctagttgaacacatctgg

SEQ ID NO: 28 - DNA, 39 bp, Artificial Sequence
  Description of Artificial Sequence Synthetic primer
aacacctgagtaggactagactagtatttagccttggag

SEQ ID NO: 29 - DNA, 19 bp, Artificial Sequence
  Description of Artificial Sequence Synthetic primer
ccctgtatcatccatcttt

SEQ ID NO: 30 - DNA, 20 bp, Artificial Sequence
  Description of Artificial Sequence Synthetic primer
tgaaacctagtctgaatctg

SEQ ID NO: 31 - DNA, 19 bp, Artificial Sequence
  Description of Artificial Sequence Synthetic primer
cgtaacagctcaccaatcg

SEQ ID NO: 32 - DNA, 18 bp, Artificial Sequence
  Description of Artificial Sequence Synthetic primer
tacgcagttacgcctcaa

SEQ ID NO: 33 - DNA, 39 bp, Artificial Sequence
  Description of Artificial Sequence Synthetic primer
cgctccttccggtacacctttcgataagacacgcggtag

SEQ ID NO: 34 - DNA, 39 bp, Artificial Sequence
  Description of Artificial Sequence Synthetic primer
aatctccctcgccgtaagccgactaacccagggaagacg

SEQ ID NO: 35 - DNA, 22 bp, Artificial Sequence
  Description of Artificial Sequence Synthetic primer
ctctgctgaatactacgctctc

SEQ ID NO: 36 - DNA, 20 bp, Artificial Sequence
  Description of Artificial Sequence Synthetic primer
caaggtttccagggtcaagc

SEQ ID NO: 37 - DNA, 19 bp, Artificial Sequence
  Description of Artificial Sequence Synthetic primer
ccaaggtttccagggtcaa

SEQ ID NO: 38 - DNA, 20 bp, Artificial Sequence
  Description of Artificial Sequence Synthetic primer
ccgaagattccccttgatcg

SEQ ID NO: 39 - DNA, 41 bp, Artificial Sequence
  Description of Artificial Sequence Synthetic primer
ctgctccatcgtctacgcagtttgctcgtcttccctgggtt

SEQ ID NO: 40 - DNA, 43 bp, Artificial Sequence
  Description of Artificial Sequence Synthetic primer
acggagtaagttaagcacgcggtgcggatttgcctactaaccg

SEQ ID NO: 41 - DNA, 19 bp, Artificial Sequence
  Description of Artificial Sequence Synthetic primer
ctcaacttaggggccgact

SEQ ID NO: 42 - DNA, 22 bp, Artificial Sequence
  Description of Artificial Sequence Synthetic primer
acgattggaagagtccgtagag

SEQ ID NO: 43 - DNA, 18 bp, Artificial Sequence
  Description of Artificial Sequence Synthetic primer
tatgcaaagcgacacctg

SEQ ID NO: 44 - DNA, 19 bp, Artificial Sequence
  Description of Artificial Sequence Synthetic primer
ttagccttggagagtggtc

SEQ ID NO: 45 - DNA, 42 bp, Artificial Sequence
  Description of Artificial Sequence Synthetic primer
tcctcaatcctacaaccccgagcgaagagattccctgtgtag

SEQ ID NO: 46 - DNA, 42 bp, Artificial Sequence
  Description of Artificial Sequence Synthetic primer
gggtgatagtcccgtagacgaacgtgtctagtcctactcagg

SEQ ID NO: 47 - DNA, 21 bp, Artificial Sequence
  Description of Artificial Sequence Synthetic primer
gagagaaagaccgacctcaac

SEQ ID NO: 48 - DNA, 19 bp, Artificial Sequence
  Description of Artificial Sequence Synthetic primer
agagattccctgtgtagcg

SEQ ID NO: 49 - DNA, 20 bp, Artificial Sequence
  Description of Artificial Sequence Synthetic primer
ccttcacagtactggttcac

SEQ ID NO: 50 - DNA, 40 bp, Artificial Sequence
  Description of Artificial Sequence Synthetic primer
ctctcctttcgtctacgggactaaccgagctgataaggct

SEQ ID NO: 51 - DNA, 40 bp, Artificial Sequence
  Description of Artificial Sequence Synthetic primer
aagaccgacctcaacacctgattagccttggagagtggtc

SEQ ID NO: 52 - DNA, 22 bp, Artificial Sequence
  Description of Artificial Sequence Synthetic primer
gttcaactaggagtcctgatcc

SEQ ID NO: 53 - DNA, 22 bp, Artificial Sequence
  Description of Artificial Sequence Synthetic primer
gtaggactagacacgtgaaacc

SEQ ID NO: 54 - DNA, 22 bp, Artificial Sequence
  Description of Artificial Sequence Synthetic primer
catgctgaatacataggtatgc

SEQ ID NO: 55 - DNA, 20 bp, Artificial Sequence
  Description of Artificial Sequence Synthetic primer
tctagtcctactcaggtgtt

SEQ ID NO: 56 - DNA, 44 bp, Artificial Sequence
  Description of Artificial Sequence Synthetic primer
tcctttatcctcaatcctacaacccatcgaagagattccctgtg

SEQ ID NO: 57 - DNA, 45 bp, Artificial Sequence
  Description of Artificial Sequence Synthetic primer
actcctagttgaacacatctggaatctttctctcctttcgtctac

SEQ ID NO: 58 - DNA, 22 bp, Artificial Sequence
  Description of Artificial Sequence Synthetic primer
tggatgatacagggtgatagtc

SEQ ID NO: 59 - DNA, 24 bp, Artificial Sequence
  Description of Artificial Sequence Synthetic primer
gggttgtaggattgaggataaagg

SEQ ID NO: 60 - DNA, 24 bp, Artificial Sequence
  Description of Artificial Sequence Synthetic primer
ggttcactatcggtcattgactag

SEQ ID NO: 61 - DNA, 48 bp, Artificial Sequence; identical to SEQ ID NO: 21
  Description of Artificial Sequence Synthetic primer

SEQ ID NO: 62 - DNA, 44 bp, Artificial Sequence; identical to SEQ ID NO: 22
  Description of Artificial Sequence Synthetic primer

SEQ ID NO: 63 - DNA, 25 bp, Artificial Sequence
  Description of Artificial Sequence Synthetic primer
caccctgtatcatccatctttccag

SEQ ID NO: 64 - DNA, 24 bp, Artificial Sequence
  Description of Artificial Sequence Synthetic primer
cgtgaaacctagtctgaatctggg

SEQ ID NO: 65 - DNA, 25 bp, Artificial Sequence
  Description of Artificial Sequence Synthetic primer
cgaactgaaacatcttagtaagcag

SEQ ID NO: 66 - DNA, 21 bp, Artificial Sequence
  Description of Artificial Sequence Synthetic primer
ctcctttcgtctacgggacta

SEQ ID NO: 67 - DNA, 49 bp, Artificial Sequence
  Description of Artificial Sequence Synthetic primer
atcagctcggtttaggctattcccgaaaagaaatcgaagagattccctg

SEQ ID NO: 68 - DNA, 49 bp, Artificial Sequence
  Description of Artificial Sequence Synthetic primer
gctcggggttgtaggattgaggatacctgtatcatccatctttccagat

SEQ ID NO: 69 - DNA, 17 bp, Artificial Sequence
  Description of Artificial Sequence Synthetic primer
ctttcgctcgccgctac

SEQ ID NO: 70 - DNA, 24 bp, Artificial Sequence
  Description of Artificial Sequence Synthetic primer
ggatcaggactcctagttgaacac

SEQ ID NO: 71 - DNA, 18 bp, Artificial Sequence
  Description of Artificial Sequence Synthetic primer
cttacaagcggtcggaga

SEQ ID NO: 72 - DNA, 20 bp, Artificial Sequence
  Description of Artificial Sequence Synthetic primer
caggtactagttcggtcctc

SEQ ID NO: 73 - DNA, 39 bp, Artificial Sequence
  Description of Artificial Sequence Synthetic primer
cccttaacctcgccgtttagccccgtaagggtcaaggtt

SEQ ID NO: 74 - DNA, 38 bp, Artificial Sequence
  Description of Artificial Sequence Synthetic primer
ccggagcgaaagcgagtttgctcacttggtttcgtgtc

SEQ ID NO: 75 - DNA, 18 bp, Artificial Sequence
  Description of Artificial Sequence Synthetic primer
tccctggctcatcatgca

SEQ ID NO: 76 - DNA, 20 bp, Artificial Sequence
  Description of Artificial Sequence Synthetic primer
gagcgaagagtcgtttggtt

SEQ ID NO: 77 - DNA, 18 bp, Artificial Sequence
  Description of Artificial Sequence Synthetic primer
ggagcaattgtttcgacg

SEQ ID NO: 78 - DNA, 18 bp, Artificial Sequence
  Description of Artificial Sequence Synthetic primer
tgtctcagtcccagtgtt

SEQ ID NO: 79 - DNA, 40 bp, Artificial Sequence
  Description of Artificial Sequence Synthetic primer
gcccaaatatcgccacattcgggcggaagggttagtaatg

SEQ ID NO: 80 - DNA, 42 bp, Artificial Sequence
  Description of Artificial Sequence Synthetic primer
gacctttcggttaagggagagtcgacgtcatagccttggtag

SEQ ID NO: 81 - DNA, 20 bp, Artificial Sequence
  Description of Artificial Sequence Synthetic primer
cgtttccaaccgttattccc

SEQ ID NO: 82 - DNA, 18 bp, Artificial Sequence
  Description of Artificial Sequence Synthetic primer
agttggtggggtaaaggc

SEQ ID NO: 83 - DNA, 19 bp, Artificial Sequence
  Description of Artificial Sequence Synthetic primer
ttagtggcggaagggttag

SEQ ID NO: 84 - DNA, 18 bp, Artificial Sequence
  Description of Artificial Sequence Synthetic primer
tctcaatccgcctagacg

SEQ ID NO: 85 - DNA, 42 bp, Artificial Sequence
  Description of Artificial Sequence Synthetic primer
aacgttactcggatgcccaaatggaataacggttggaaacgg

SEQ ID NO: 86 - DNA, 42 bp, Artificial Sequence
  Description of Artificial Sequence Synthetic primer
aggacctttcggttaagggagatagccttggtaggcctttac

SEQ ID NO: 87 - DNA, 21 bp, Artificial Sequence
  Description of Artificial Sequence Synthetic primer
atcgccacattcggtattagc

SEQ ID NO: 88 - DNA, 22 bp, Artificial Sequence
  Description of Artificial Sequence Synthetic primer
gtgatatcagctagttggtggg

SEQ ID NO: 89 - DNA, 15 bp, Artificial Sequence
  Description of Artificial Sequence Synthetic primer
gaacggagcaattgt

SEQ ID NO: 90 - DNA, 20 bp, Artificial Sequence
  Description of Artificial Sequence Synthetic primer
ctgatatcacatagactctc

SEQ ID NO: 91 - DNA, 36 bp, Artificial Sequence
  Description of Artificial Sequence Synthetic primer
ccgtttccaaccgttattctcgacgattgtttagtg

SEQ ID NO: 92 - DNA, 37 bp, Artificial Sequence
  Description of Artificial Sequence Synthetic primer
taccgaatgtggcgatatttcgaaaggtcctaagatc

SEQ ID NO: 93 - DNA, 20 bp, Artificial Sequence
  Description of Artificial Sequence Synthetic primer
ctatgcattactaacccttc

SEQ ID NO: 94 - DNA, 19 bp, Artificial Sequence
  Description of Artificial Sequence Synthetic primer
catccgagtaacgttaaag

SEQ ID NO: 95 - DNA, 18 bp, Artificial Sequence
  Description of Artificial Sequence Synthetic primer
ccggctaaggtcccaaat

SEQ ID NO: 96 - DNA, 19 bp, Artificial Sequence
  Description of Artificial Sequence Synthetic primer
tcgcacttctgatacctcc

SEQ ID NO: 97 - DNA, 40 bp, Artificial Sequence
  Description of Artificial Sequence Synthetic primer
tcgaccagtgagctattacgctgaagtgggaaggcacaga

SEQ ID NO: 98 - DNA, 41 bp, Artificial Sequence
  Description of Artificial Sequence Synthetic primer
ctataaccgaagctgcggatgcatcagcctacagaacgctc

SEQ ID NO: 99 - DNA, 21 bp, Artificial Sequence
  Description of Artificial Sequence Synthetic primer
gcttctaagccaacatcctgg

SEQ ID NO: 100 - DNA, 19 bp, Artificial Sequence
  Description of Artificial Sequence Synthetic primer
cggtttaccggcatggtag

SEQ ID NO: 101 - DNA, 20 bp, Artificial Sequence
  Description of Artificial Sequence Synthetic primer
agagaactcgggagaaggaa

SEQ ID NO: 102 - DNA, 20 bp, Artificial Sequence
  Description of Artificial Sequence Synthetic primer
tcgctaccttaggaccgtta

SEQ ID NO: 103 - DNA, 43 bp, Artificial Sequence
  Description of Artificial Sequence Synthetic primer
cagccacctattctctgcgaccggagaaggtatgccctctaag

SEQ ID NO: 104 - DNA, 40 bp, Artificial Sequence
  Description of Artificial Sequence Synthetic primer
cgtatagggtgtaacgcctgccggcttcgatccgatgctt

SEQ ID NO: 105 - DNA, 19 bp, Artificial Sequence
  Description of Artificial Sequence Synthetic primer
ggcttacggagcaagtcct

SEQ ID NO: 106 - DNA, 20 bp, Artificial Sequence
  Description of Artificial Sequence Synthetic primer
cggtgccggaaggttaattg

SEQ ID NO: 107 - DNA, 18 bp, Artificial Sequence
  Description of Artificial Sequence Synthetic primer
ggagaaggaactcggcaa

SEQ ID NO: 108 - DNA, 18 bp, Artificial Sequence
  Description of Artificial Sequence Synthetic primer
cttcgatccgatgcttgc

SEQ ID NO: 109 - DNA, 41 bp, Artificial Sequence
  Description of Artificial Sequence Synthetic primer
agccacctattctctgcgaccggagaaggtatgccctctaa

SEQ ID NO: 110 - DNA, 40 bp, Artificial Sequence
  Description of Artificial Sequence Synthetic primer
gagcactcttgccaacacgaacaattaaccttccggcacc

SEQ ID NO: 111 - DNA, 22 bp, Artificial Sequence
  Description of Artificial Sequence Synthetic primer
cttacggagcaagtccttaacc

SEQ ID NO: 112 - DNA, 20 bp, Artificial Sequence
  Description of Artificial Sequence Synthetic primer
gtatagggtgtaacgcctgc

SEQ ID NO: 113 - DNA, 38 bp, Artificial Sequence
  Description of Artificial Sequence Synthetic primer
agcactctgccaacacgaaccacggccttccaattaac

SEQ ID NO: 114 - DNA, 20 bp, Artificial Sequence
  Description of Artificial Sequence Synthetic primer
gtatagggtgtcacgcctgc

SEQ ID NO: 115 - DNA, 19 bp, Artificial Sequence
  Description of Artificial Sequence Synthetic primer
agagaataggtggctgcga

SEQ ID NO: 116 - DNA, 18 bp, Artificial Sequence
  Description of Artificial Sequence Synthetic primer
agacagtgtggccatcgt

SEQ ID NO: 117 - DNA, 47 bp, Artificial Sequence
  Description of Artificial Sequence Synthetic primer
gcaggcgtcacaccctatacggtttattaaaaacacagcactctgcc

SEQ ID NO: 118 - DNA, 37 bp, Artificial Sequence
  Description of Artificial Sequence Synthetic primer
cggtaaacggcggccgtaaattcgtgcgggtcggaac

SEQ ID NO: 119 - DNA, 22 bp, Artificial Sequence
  Description of Artificial Sequence Synthetic primer
ctatacgtccactttcgtgttg

SEQ ID NO: 120 - DNA, 21 bp, Artificial Sequence
  Description of Artificial Sequence Synthetic primer
taacggtcctaaggtagcgaa

SEQ ID NO: 121 - DNA, 18 bp, Artificial Sequence
  Description of Artificial Sequence Synthetic primer
gagcgcaacccttgtcat

SEQ ID NO: 122 - DNA, 19 bp, Artificial Sequence
  Description of Artificial Sequence Synthetic primer
tccgacttcatgcactcga

SEQ ID NO: 123 - DNA, 42 bp, Artificial Sequence
  Description of Artificial Sequence Synthetic primer
gaggacttgacgtcatccccacgggcactctaatgagactgc

SEQ ID NO: 124 - DNA, 42 bp, Artificial Sequence
  Description of Artificial Sequence Synthetic primer
atggtcggtacagagggtagccagtgcaatccggactacgat

SEQ ID NO: 125 - DNA, 20 bp, Artificial Sequence
  Description of Artificial Sequence Synthetic primer
cttcctccggcttgtcaccg

SEQ ID NO: 126 - DNA, 23 bp, Artificial Sequence
  Description of Artificial Sequence Synthetic primer
aggcggagccaatctcacaaaac

SEQ ID NO: 127 - DNA, 18 bp, Artificial Sequence
  Description of Artificial Sequence Synthetic primer
atgccgaaagctatttgg

SEQ ID NO: 128 - DNA, 16 bp, Artificial Sequence
  Description of Artificial Sequence Synthetic primer
gccgtctttcgggtta

SEQ ID NO: 129 - DNA, 38 bp, Artificial Sequence
  Description of Artificial Sequence Synthetic primer
cttcttccaacgtaaccgcatagtggcggaaggtatcg

SEQ ID NO: 130 - DNA, 33 bp, Artificial Sequence
  Description of Artificial Sequence Synthetic primer
gccggtaaaccgtctgcccgaagtcctgtaccg

SEQ ID NO: 131 - DNA, 16 bp, Artificial Sequence
  Description of Artificial Sequence Synthetic primer
cgtccaacgccttagc

SEQ ID NO: 132 - DNA, 16 bp, Artificial Sequence
  Description of Artificial Sequence Synthetic primer
ttgccgaaggactggt

SEQ ID NO: 133 - DNA, 15 bp, Artificial Sequence
  Description of Artificial Sequence Synthetic primer
ctggtggcggaaggt

SEQ ID NO: 134 - DNA, 15 bp, Artificial Sequence
  Description of Artificial Sequence Synthetic primer
atccgttcgccgtct

SEQ ID NO: 135 - DNA, 31 bp, Artificial Sequence
  Description of Artificial Sequence Synthetic primer
ggcacggcttcttccaaatcgcggctaaggc

SEQ ID NO: 136 - DNA, 31 bp, Artificial Sequence
  Description of Artificial Sequence Synthetic primer
tttgccgaaggactggtgatacgccgcctgc

SEQ ID NO: 137 - DNA, 19 bp, Artificial Sequence
  Description of Artificial Sequence Synthetic primer
gtaaccgcatattcgtcca

SEQ ID NO: 138 - DNA, 17 bp, Artificial Sequence
  Description of Artificial Sequence Synthetic primer
tcggtacaggacttcgg

SEQ ID NO: 139 - DNA, 21 bp, Artificial Sequence
  Description of Artificial Sequence Synthetic primer
aagctatttggaaaaactggt

SEQ ID NO: 140 - DNA, 37 bp, Artificial Sequence
  Description of Artificial Sequence Synthetic primer
cttcttccaacgtaaccgcatagaaggtatcgcggct

SEQ ID NO: 141 - DNA, 30 bp, Artificial Sequence
  Description of Artificial Sequence Synthetic primer
cgtgccggtaaaccgtcgccgaagtcctgt

SEQ ID NO: 142 - DNA, 20 bp, Artificial Sequence
  Description of Artificial Sequence Synthetic primer
catattcgtccaacgcctta

SEQ ID NO: 143 - DNA, 15 bp, Artificial Sequence
  Description of Artificial Sequence Synthetic primer
tgccgaaggactggt

SEQ ID NO: 144 - DNA, 18 bp, Artificial Sequence
  Description of Artificial Sequence Synthetic primer
ttggacgaatatgcggtt

SEQ ID NO: 145 - DNA, 17 bp, Artificial Sequence
  Description of Artificial Sequence Synthetic primer
ccgtctgaaagcccaaa

SEQ ID NO: 146 - DNA, 34 bp, Artificial Sequence
  Description of Artificial Sequence Synthetic primer
agatacgccgcctgctgcgttggaagaagccgtg

SEQ ID NO: 147 - DNA, 35 bp, Artificial Sequence
  Description of Artificial Sequence Synthetic primer
tggaactggcggactgcggcgatattgtcctccac

SEQ ID NO: 148 - DNA, 18 bp, Artificial Sequence
  Description of Artificial Sequence Synthetic primer
ccgaagtcctgtaccgac

SEQ ID NO: 149 - DNA, 17 bp, Artificial Sequence
  Description of Artificial Sequence Synthetic primer
cgttaccgccttggaca

SEQ ID NO: 150 - DNA, 19 bp, Artificial Sequence
  Description of Artificial Sequence Synthetic primer
ttggacgaatatgcggtta

SEQ ID NO: 151 - DNA, 33 bp, Artificial Sequence
  Description of Artificial Sequence Synthetic primer
cgccgtctttcgggttaaggagccgtgccggta

SEQ ID NO: 152 - DNA, 41 bp, Artificial Sequence
  Description of Artificial Sequence Synthetic primer
gcatattttggaactggcggacgatattgtcctccacacgc

SEQ ID NO: 153 - DNA, 15 bp, Artificial Sequence
  Description of Artificial Sequence Synthetic primer
acgccgaagtcctgt

SEQ ID NO: 154 - DNA, 16 bp, Artificial Sequence
  Description of Artificial Sequence Synthetic primer
cgttaccgccttggac

SEQ ID NO: 155 - DNA, 21 bp, Artificial Sequence
  Description of Artificial Sequence Synthetic primer
gcaatgccgaaagctatttgg

SEQ ID NO: 156 - DNA, 20 bp, Artificial Sequence
  Description of Artificial Sequence Synthetic primer
aaatccgttcgccgtctttc

SEQ ID NO: 157 - DNA, 41 bp, Artificial Sequence
  Description of Artificial Sequence Synthetic primer
ggcttcttccaacgtaaccgcattggtggcggaaggtatcg

SEQ ID NO: 158 - DNA, 38 bp, Artificial Sequence
  Description of Artificial Sequence Synthetic primer
tgcccggttttgccgaaggtaaggagatacgccgcctg

SEQ ID NO: 159 - DNA, 20 bp, Artificial Sequence
  Description of Artificial Sequence Synthetic primer
attcgtccaacgccttagcc

SEQ ID NO: 160 - DNA, 21 bp, Artificial Sequence
  Description of Artificial Sequence Synthetic primer
actggtgtcggtacaggactt

SEQ ID NO: 161 - DNA, 27 bp, Artificial Sequence
  Description of Artificial Sequence Synthetic primer
ccgtctgcccggtcgccgaagtcctgt

SEQ ID NO: 162 - DNA, 19 bp, Artificial Sequence
  Description of Artificial Sequence Synthetic primer
ttggaacagaggcattggt

SEQ ID NO: 163 - DNA, 21 bp, Artificial Sequence
  Description of Artificial Sequence Synthetic primer
caacttgtttatccgagccag

SEQ ID NO: 164 - DNA, 42 bp, Artificial Sequence
  Description of Artificial Sequence Synthetic primer
gctgactaatgcgcgagcaggcattctgatgtagccattgaa

SEQ ID NO: 165 - DNA, 44 bp, Artificial Sequence
  Description of Artificial Sequence Synthetic primer
ggttatcgtgctcaagcacaaggtctgtttggctaggagtttga

SEQ ID NO: 166 - DNA, 22 bp, Artificial Sequence
  Description of Artificial Sequence Synthetic primer
accagacattgcatttgcttgt

SEQ ID NO: 167 - DNA, 18 bp, Artificial Sequence
  Description of Artificial Sequence Synthetic primer
tgcctgaaggtgtctccg

SEQ ID NO: 168 - DNA, 20 bp, Artificial Sequence
  Description of Artificial Sequence Synthetic primer
gcattctgatgtagccattg

SEQ ID NO: 169 - DNA, 18 bp, Artificial Sequence
  Description of Artificial Sequence Synthetic primer
ctctgtttggctaggagt

SEQ ID NO: 170 - DNA, 44 bp, Artificial Sequence
  Description of Artificial Sequence Synthetic primer
tcttctcaaaaccttctgaaacaccaagcaaatgcaatgtctgg

SEQ ID NO: 171 - DNA, 38 bp, Artificial Sequence
  Description of Artificial Sequence Synthetic primer
cgtgctcaagcacaaggtaattgaacggagacaccttc

SEQ ID NO: 172 - DNA, 21 bp, Artificial Sequence
  Description of Artificial Sequence Synthetic primer
accatattgctgactaatgcg

SEQ ID NO: 173 - DNA, 22 bp, Artificial Sequence
  Description of Artificial Sequence Synthetic primer
atccgatcgtatatgaaatgcc

SEQ ID NO: 174 - DNA, 19 bp, Artificial Sequence
  Description of Artificial Sequence Synthetic primer
gcagtaaacaagcaaatgc

SEQ ID NO: 175 - DNA, 43 bp, Artificial Sequence
  Description of Artificial Sequence Synthetic primer
tcttctcaaaaccttctgaaacaccaatgtctggtactgctcg

SEQ ID NO: 176 - DNA, 43 bp, Artificial Sequence
  Description of Artificial Sequence Synthetic primer
cttctcaaaaccttctgaaacacctaatgtctggtactgctcg

SEQ ID NO: 177 - DNA, 44 bp, Artificial Sequence
  Description of Artificial Sequence Synthetic primer
tcttctcaaaaccttctgaaacaccgcagtaaacaagcaaatgc

SEQ ID NO: 178 - DNA, 19 bp, Artificial Sequence
  Description of Artificial Sequence Synthetic primer
ttggaacagaggcattggt

SEQ ID NO: 179 - DNA, 21 bp, Artificial Sequence
  Description of Artificial Sequence Synthetic primer
caacttgtttatccgagccag

SEQ ID NO: 180 - DNA, 38 bp, Artificial Sequence
  Description of Artificial Sequence Synthetic probe
cgcgatcaggactcctagttgaacacatctggatcgcg

SEQ ID NO: 181 - DNA, 33 bp, Artificial Sequence
  Description of Artificial Sequence Synthetic probe
cgtccaggactcctagttgaacacatctggacg

SEQ ID NO: 182 - DNA, 40 bp, Artificial Sequence
  Description of Artificial Sequence Synthetic probe
cgcgactcaggactcctagttgaacacatctggagtcgcg

SEQ ID NO: 183 - DNA, 37 bp, Artificial Sequence
  Description of Artificial Sequence Synthetic probe
cgcgagtaggattgaggataaaggatcaggactcgcg

SEQ ID NO: 184 - DNA, 38 bp, Artificial Sequence
  Description of Artificial Sequence Synthetic probe
cgcgcacatctggaaagatggatgatacagggtgcgcg

SEQ ID NO: 185 - DNA, 42 bp, Artificial Sequence
  Description of Artificial Sequence Synthetic probe
cgcgatccggataaaggatcaggactcctagttgggatcgcg

SEQ ID NO: 186 - DNA, 34 bp, Artificial Sequence
  Description of Artificial Sequence Synthetic probe
cgcgatcagaccgacctcaacacctgagatcgcg

SEQ ID NO: 187 - DNA, 33 bp, Artificial Sequence
  Description of Artificial Sequence Synthetic probe
cgcagtgagagaaagaccgacctcaacactgcg

SEQ ID NO: 188 - DNA, 34 bp, Artificial Sequence
  Description of Artificial Sequence Synthetic probe
cgcgatcctgtgtagcggcgagcgaaagatcgcg

SEQ ID NO: 189 - DNA, 35 bp, Artificial Sequence
  Description of Artificial Sequence Synthetic probe
cgcgatcagatccatggcataagtaacggatcgcg

SEQ ID NO: 190 - DNA, 41 bp, Artificial Sequence
  Description of Artificial Sequence Synthetic probe
cgcgatcggtgaagatccatggcataagtaacgcgatcgcg

SEQ ID NO: 191 - DNA, 44 bp, Artificial Sequence
  Description of Artificial Sequence Synthetic probe
cgcgatccatggcataagtaacgataaagggagtgaggatcgcg

SEQ ID NO: 192 - DNA, 37 bp, Artificial Sequence
  Description of Artificial Sequence Synthetic probe
cgcgatcatgacggagtaagttaagcacgcgatcgcg

SEQ ID NO: 193 - DNA, 36 bp, Artificial Sequence
  Description of Artificial Sequence Synthetic probe
cgcgatgtctggaaagatggatgatacagcatcgcg

SEQ ID NO: 194 - DNA, 36 bp, Artificial Sequence
  Description of Artificial Sequence Synthetic probe
cgcgatctgaggataaaggatcaggactcgatcgcg

SEQ ID NO: 195 - DNA, 33 bp, Artificial Sequence
  Description of Artificial Sequence Synthetic probe
cgcgatccctgtgtagcggcgagcgagatcgcg

SEQ ID NO: 196 - DNA, 33 bp, Artificial Sequence
  Description of Artificial Sequence Synthetic probe
cgcgatcgcaaaaggcacgccgtcaagatcgcg

SEQ ID NO: 197 - DNA, 34 bp, Artificial Sequence
  Description of Artificial Sequence Synthetic probe
cggctcggggttgtaggattgaggatacgagccg

SEQ ID NO: 198 - DNA, 35 bp, Artificial Sequence
  Description of Artificial Sequence Synthetic probe
ccggagcctacaaccccgagccttatcagctccgg

SEQ ID NO: 199 - DNA, 33 bp, Artificial Sequence
  Description of Artificial Sequence Synthetic probe
cgcgcagctcggtttaggctattcccctgcgcg

SEQ ID NO: 200 - DNA, 31 bp, Artificial Sequence
  Description of Artificial Sequence Synthetic probe
cgcggtctctcctttcgtctacgggaccgcg

SEQ ID NO: 201 - DNA, 37 bp, Artificial Sequence
  Description of Artificial Sequence Synthetic probe
cgcgatcatccgagtaacgttaaagaaggggatcgcg

SEQ ID NO: 202 - DNA, 35 bp, Artificial Sequence
  Description of Artificial Sequence Synthetic probe
cgcgatctggcgatatttgggcatccgagatcgcg

SEQ ID NO: 203 - DNA, 35 bp, Artificial Sequence
  Description of Artificial Sequence Synthetic probe
cgcgatccacgagcactcttgccaacacgatcgcg

SEQ ID NO: 204 - DNA, 39 bp, Artificial Sequence
  Description of Artificial Sequence Synthetic probe
cgcgatcggagcactcttgccaacacgaaagcgatcgcg

SEQ ID NO: 205 - DNA, 34 bp, Artificial Sequence
  Description of Artificial Sequence Synthetic probe
cgcgatcagcactctgccaacacgaaagatcgcg

SEQ ID NO: 206 - DNA, 38 bp, Artificial Sequence
  Description of Artificial Sequence Synthetic probe
cgcgatccgtcctgcgcggaagatgtaacgggatcgcg

SEQ ID NO: 207 - DNA, 32 bp, Artificial Sequence
  Description of Artificial Sequence Synthetic probe
cgcgatcctgcgcggaagatgtaacgatcgcg

SEQ ID NO: 208 - DNA, 32 bp, Artificial Sequence
  Description of Artificial Sequence Synthetic probe
cgcgatcgcacgagcactcttgcccgatcgcg

SEQ ID NO: 209 - DNA, 34 bp, Artificial Sequence
  Description of Artificial Sequence Synthetic probe
cgcgatcacttgtttattaaaaacacggatcgcg

SEQ ID NO: 210 - DNA, 41 bp, Artificial Sequence
  Description of Artificial Sequence Synthetic probe
cgcgacccgacttgtttattaaaaacacgagcacggtcgcg

SEQ ID NO: 211 - DNA, 43 bp, Artificial Sequence
  Description of Artificial Sequence Synthetic probe
cgcgaccccgacttgtttattaaaaacacgagcacgggtcgcg

SEQ ID NO: 212 - DNA, 36 bp, Artificial Sequence
  Description of Artificial Sequence Synthetic probe
cgcgatcagcagccatcatttaaagaaaggatcgcg

SEQ ID NO: 213 - DNA, 35 bp, Artificial Sequence
  Description of Artificial Sequence Synthetic probe
caggccggttttgccgaaggactggtgtcggcctg

SEQ ID NO: 214 - DNA, 33 bp, Artificial Sequence
  Description of Artificial Sequence Synthetic probe
cgcgaaggactggtgtcggtacaggacttcgcg

SEQ ID NO: 215 - DNA, 24 bp, Artificial Sequence
  Description of Artificial Sequence Synthetic probe
ctagcgaaggactggtgtcgctag

SEQ ID NO: 216 - DNA, 33 bp, Artificial Sequence
  Description of Artificial Sequence Synthetic probe
cgcgatgtttgccgaaggactggtgtcatcgcg

SEQ ID NO: 217 - DNA, 38 bp, Artificial Sequence
  Description of Artificial Sequence Synthetic probe
cgcgatcgaagaaattacaattgatgggcgtgatcgcg

SEQ ID NO: 218 - DNA, 37 bp, Artificial Sequence
  Description of Artificial Sequence Synthetic probe
cgcgatcgaagaaattacaattgataggcggatcgcg

(19) United States
(12) Patent Application Publication
(10) Pub. No.: US 2021/0254139 A1
(43) Pub. Date: Aug. 19, 2021

(54) PROBE DETECTION OF LOOP-MEDIATED AMPLIFICATION PRODUCTS

(71) Applicant: Talis Biomedical Corporation, Menlo Park, CA (US)

(72) Inventors: Andrea C. Dedent, San Francisco, CA (US); Matthew B. Lee, San Jose, CA (US); Hedia Maamar, San Jose, CA (US)

(21) Appl. No.: 16/349,190

(22) PCT Filed: Nov. 13, 2017
(86) PCT No.: PCT/US2017/061403

§ 371 Date: May 10, 2019

Related U.S. Application Data
(60) Provisional application No. 62/420,488, filed on Nov. 10, 2016.
(60) Provisional application No. 62/420,496, filed on Nov. 10, 2016.

(51) Int. Cl. C12Q 1/6813; C12Q 1/6816

(52) U.S. Cl. CPC C12Q 1/6813; C12Q 1/6816

(57) ABSTRACT

Disclosed herein are methods and compositions for detecting amplicon generated using loop-mediated amplification (LAMP). In particular, the invention relates to compositions comprising molecular beacons and/or LAMP primers and methods for generating and detecting LAMP amplicons. In particular, molecular beacons that bind to novel regions of a LAMP amplicon, and methods of using these probes, are provided.

### CROSS-REFERENCE TO RELATED APPLICATIONS

This application claims the benefit of U.S. Provisional Application No. 62/420,488, filed Nov. 10, 2016, and U.S. Provisional Application No. 62/420,496, filed Nov. 10, 2016, the contents of which are each incorporated by reference in their entirety.

### STATEMENT REGARDING FEDERALLY SPONSORED RESEARCH OR DEVELOPMENT

This invention was made with government support under contract number HR0011-11-2-0006 awarded by the Department of Defense. The government has certain rights in the invention.

### FIELD OF THE INVENTION

The present invention relates to the fields of molecular biology and nucleic acid chemistry. The invention provides methods and reagents for detecting amplicon generated using loop-mediated amplification (LAMP). In particular, the invention relates to compositions comprising molecular beacons and/or LAMP primers and methods for generating and detecting LAMP amplicons.

### BACKGROUND

Loop mediated isothermal amplification (LAMP) is an isothermal nucleic acid amplification technique that is a rapid and reliable sequence-specific real-time detection technique for low-cost or point-of-care diagnostics. The technique can be coupled with reverse transcription (RT-LAMP) for detection of RNA targets. A major challenge for LAMP in point-of-care applications is multiplexed detection to distinguish multiple targets in a single reaction, e.g., for syndromic panels or variant strains of pathogens. Also, some reactions require a high sensitivity and specificity, while maintaining a fast detection time from the start of an amplification reaction.

Measurement of the presence or absence of LAMP amplicons is generally performed via non-sequence specific techniques, such as fluorescent dye intercalation into dsDNA, bioluminescence through pyrophosphate conversion, or turbidity detection of precipitated magnesium pyrophosphate. These methods are limited to measurement of a single product, and have limited sensitivity and specificity.

Oligonucleotide probes have been used for sequence-specific detection of LAMP amplification (Tanner et al., Simultaneous multiple target detection in real-time loop-mediated isothermal amplification, Biotechniques 2012, 53, 81-89). However, these probes specifically target only the loop region of the amplicon, specifically, the F2 region. This is consistent with primers used to generate amplicons during LAMP, which also bind to loop regions of the amplicon to continue amplification during LAMP. However, there are some drawbacks with the use of oligonucleotide probes, as they may interfere with LAMP amplification, and the sequences in the loop region may not provide the desired specificity, sensitivity, and reaction speed needed for an assay using oligonucleotide probes for detection. What is needed therefore, are new compositions and methods for LAMP amplicon detection that are rapid, sensitive and specific, and facilitate multiplexed detection.

### SUMMARY OF THE INVENTION

Provided herein, according to some embodiments, is a composition comprising a LAMP primer set and an oligonucleotide probe comprising a detectable label, wherein the LAMP primer set, when used in a LAMP amplification reaction in the presence of a target nucleic acid, generates an amplicon comprising a first region or a second region, wherein the first region comprises a B1 region and an F1c region and extends from the 5′ end of the B1 region to the 3′ end of the F1c region, and wherein the second region comprises an F1 region and a B1c region and extends from the 5′ end of the F1 region to the 3′ end of the B1c region, and wherein the amplicon further comprises a probe target sequence; and wherein the oligonucleotide probe binds specifically to the amplicon at the probe target sequence, wherein the probe target sequence overlaps with the first region or the second region. In some embodiments, the composition also comprises the target nucleic acid.

In some embodiments, the target nucleic acid comprises a B2 and a B1 region in this order from a 5′ terminal side, and an F2c and an F1c region in this order from a 3′ terminal side.

In some embodiments, the LAMP primer set comprises: a forward inner primer comprising an F1c region and an F2 region, wherein the F1c region of the forward inner primer comprises a sequence substantially identical to the F1c region of the target nucleic acid and wherein the F2 region of the forward inner primer comprises a sequence substantially complementary to the F2c region of the target nucleic acid; and a backward inner primer comprising a B1c region and a B2 region, wherein the B1c region of the backward inner primer comprises a sequence substantially complementary to a the B1 region of the target nucleic acid and wherein the B2 region of the backward inner primer comprises a sequence substantially identical to the B2 region of the target nucleic acid sequence.

In some embodiments, the target nucleic acid comprises an F3c region 3′ of the F2c region and a B3 region 5′ of the B2 region, and wherein the LAMP primer set further comprises a forward outer primer and a backward outer primer, wherein the forward outer primer comprises a sequence substantially complementary to the F3c region of the target nucleic acid and wherein the backward outer primer comprises a sequence substantially identical to the B3 region of the target nucleic acid.

In some embodiments, the LAMP primer set comprises a loop forward primer and a loop backward primer, wherein the loop forward primer comprises a sequence substantially identical to a sequence between the F1c and the F2c region of the target nucleic acid, and wherein the loop backward primer comprises a sequence substantially complementary to a sequence between the B1 and the B2 region of the target nucleic acid.

In some embodiments, the oligonucleotide probe comprises a sequence substantially complementary to the probe target sequence. In some embodiments, the probe target sequence overlaps the first region or the second region of the amplicon by at least 3 nucleotides. In some embodiments, the probe target sequence overlaps the first region or the second region of the amplicon by at least 7 nucleotides. In some embodiments, the probe target sequence overlaps the first region or the second region of the amplicon by at least 10 nucleotides. In some embodiments, the probe target sequence is located completely within the first region or the second region of the amplicon.

In some embodiments, the probe target sequence overlaps with at least 3 nucleotides, at least 7 nucleotides, at least 10 nucleotides, or all of the F1 region, the F1c region, the B1 region, or the B1c region of the amplicon. In some embodiments, the probe target sequence is at least 8, 9, 10, 11, 12, 13, 14, 15, 16, 17, 18, 19 or 20 nucleotides in length.

In some embodiments, the detectable label is covalently bound to a terminus of the oligonucleotide probe. In some embodiments, the detectable label is a fluorophore. In some embodiments, the oligonucleotide probe further comprises a quencher. In some embodiments, the quencher is covalently bound to a terminus of the oligonucleotide probe. In some embodiments, the detectable label is FAM and wherein the quencher is BHQ1. In some embodiments, the detectable label is ATTO 565 and wherein the quencher is BHQ1 or BHQ2. In some embodiments, the oligonucleotide probe is a molecular beacon.

Also provided herein is a method of detecting the presence or absence of a target nucleic acid in a test sample, the method comprising: mixing the test sample with a reaction mixture comprising a strand displacement DNA polymerase and a LAMP primer set; exposing the test sample to loop-mediated amplification reaction conditions to generate an amplicon from the target nucleic acid, if present in the test sample, wherein the amplicon comprises a probe target sequence; contacting the test sample with an oligonucleotide probe comprising a detectable label, wherein the oligonucleotide probe binds specifically to the amplicon at the probe target sequence, if present, wherein the probe target sequence overlaps with a first region or a second region, wherein the first region comprises a B1 region and an F1c region and extends from the 5′ end of the B1 region to the 3′ end of the F1c region, and wherein the second region comprises an F1region and a B1c region and extends from the 5′ end of the F1 region to the 3′ end of the B1c region; and detecting the presence or absence of a signal from the detectable label, wherein the presence of the signal is indicative of the presence of the target nucleic acid in the test sample.

In some embodiments, the loop-mediated amplification reaction is performed at a temperature of between about 60° C. and about 67° C. In some embodiments, the oligonucleotide probe is a molecular beacon. In some embodiments, the reaction mixture comprises a reverse transcriptase.

In some embodiments, the loop-mediated amplification reaction is performed for less than 30 minutes. In some embodiments, the loop-mediated amplification reaction is performed for less than 15 minutes. In some embodiments, the loop-mediated amplification reaction is performed for less than nine minutes.

Also provided herein is a method of detecting the presence or absence of a target nucleic acid in a test sample, the method comprising: providing a test sample suspected of comprising a target nucleic acid, wherein the test sample comprises a LAMP primer set and an oligonucleotide probe according to any of the embodiments provided herein, and a strand displacement DNA polymerase; exposing the test sample to conditions sufficient to generate an amplicon from the target nucleic acid, if present in the test sample, via a loop-mediated amplification reaction; and detecting the presence or absence of a signal from the detectable label, wherein the presence of the signal is indicative of the presence of the target nucleic acid in the test sample.

In some embodiments, provided herein is a kit comprising a LAMP primer set, an oligonucleotide probe comprising a detectable label, and instructions for use, wherein the LAMP primer set, when used in a LAMP amplification reaction in the presence of a target nucleic acid, generates an amplicon comprising a first region or a second region, wherein the first region comprises a B1 region and an F1c region and extends from the 5′ end of the B1 region to the 3′ end of the F1c region, and wherein the second region comprises an F1 region and a B1c region and extends from the 5′ end of the F1 region to the 3′ end of the B1c region, and wherein the target nucleic acid further comprises a probe target sequence; and wherein the oligonucleotide probe binds specifically to the amplicon at the probe target sequence, wherein the probe target sequence overlaps with the first region or the second region.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention.

FIG. 1 is a diagram of LAMP amplification showing regions on the target nucleic acid, primers, and amplicons used herein.

FIG. 2A is a diagram of a LAMP amplicon, according to an embodiment of the invention, and examples of oligonucleotide probe binding to selected regions of the amplicon.

FIG. 2B is a diagram of a LAMP amplicon, according to another embodiment of the invention, and examples of oligonucleotide probe binding to selected regions of the amplicon.

FIG. 3 is a diagram of RT-LAMP, according to an embodiment of the invention, and regions on the RNA target nucleic acid, cDNA, and examples of primer binding to regions of sense and antisense strands generated from RT-LAMP, according to an embodiment of the invention.

FIG. 4 is a table showing results of detection of 23S target nucleic acid from samples containing C. trachomatis using LAMP primer sets and oligonucleotide probes that bind to DS and Loop regions of the amplicon generated by the LAMP primer set from target nucleic acid. Diagrams of oligonucleotide probe binding to each amplicon are shown.

FIGS. 5A-5H are diagrams of an amplicon generated via LAMP amplification with primer set-1 directed to C. trachomatis (i.e., “CT”) 23S target nucleic acid, and embodiments of oligonucleotide probe binding to the amplicon at different probe target sequences of the amplicon, according to embodiments of the invention provided herein.

FIG. 6 is a diagram of an amplicon generated via LAMP amplification with primer set-2/set-11 directed to CT 23S target nucleic acid, and embodiments of oligonucleotide probe binding to the amplicon at a probe target sequence of the amplicon, according to an embodiment of the invention provided herein.

FIGS. 7A-7B are diagrams of an amplicon generated via LAMP amplification with primer set-3 directed to CT 23S target nucleic acid, and embodiments of oligonucleotide probe binding to the amplicon at different probe target sequences of the amplicon, according to embodiments of the invention provided herein.

FIG. 8 is a diagram of an amplicon generated via LAMP amplification with primer set-4 directed to CT 23S target nucleic acid, and embodiments of oligonucleotide probe binding to the amplicon at a probe target sequence of the amplicon, according to an embodiment of the invention provided herein.

FIGS. 9A-9B are diagrams of an amplicon generated via LAMP amplification with primer set-5/set-12 directed to CT 23S target nucleic acid, and embodiments of oligonucleotide probe binding to the amplicon at different probe target sequences of the amplicon, according to embodiments of the invention provided herein.

FIGS. 10A-10C are diagrams of an amplicon generated via LAMP amplification with primer set-37 directed to CT 23S target nucleic acid, and embodiments of oligonucleotide probe binding to the amplicon at different probe target sequences of the amplicon, according to embodiments of the invention provided herein.

FIG. 11 is a diagram of an amplicon generated via LAMP amplification with primer set-38 directed to CT 23S target nucleic acid, and embodiments of oligonucleotide probe binding to the amplicon at a probe target sequence of the amplicon, according to an embodiment of the invention provided herein.

FIG. 12 is a diagram of an amplicon generated via LAMP amplification with primer set-39 directed to CT 23S target nucleic acid, and embodiments of oligonucleotide probe binding to the amplicon at a probe target sequence of the amplicon, according to an embodiment of the invention provided herein.

FIG. 13 is a diagram of an amplicon generated via LAMP amplification with primer set-40 directed to CT 23S target nucleic acid, and embodiments of oligonucleotide probe binding to the amplicon at a probe target sequence of the amplicon, according to an embodiment of the invention provided herein.

FIGS. 14A-14B are diagrams of an amplicon generated via LAMP amplification with primer set-43 directed to CT 23S target nucleic acid, and embodiments of oligonucleotide probe binding to the amplicon at different probe target sequences of the amplicon, according to embodiments of the invention provided herein.

FIG. 15 is a diagram of an amplicon generated via LAMP amplification with primer set-44 directed to CT 23S target nucleic acid, and embodiments of oligonucleotide probe binding to the amplicon at a probe target sequence of the amplicon, according to an embodiment of the invention provided herein.

FIG. 16 is a diagram of an amplicon generated via LAMP amplification with primer set-49 directed to CT 16S target nucleic acid, and embodiments of oligonucleotide probe binding to the amplicon at a probe target sequence of the amplicon, according to an embodiment of the invention provided herein.

FIGS. 17A-17B are diagrams of an amplicon generated via LAMP amplification with primer set-59 directed to N. Gonorrhoeae (i.e., “NG”) 23S target nucleic acid, and embodiments of oligonucleotide probe binding to the amplicon at different probe target sequences of the amplicon, according to embodiments of the invention provided herein.

FIGS. 18A-18B are diagrams of an amplicon generated via LAMP amplification with primer set-60 directed to NG 23S target nucleic acid, and embodiments of oligonucleotide probe binding to the amplicon at different probe target sequences of the amplicon, according to embodiments of the invention provided herein.

FIGS. 19A-19C are diagrams of an amplicon generated via LAMP amplification with primer set-61 directed to NG 23S target nucleic acid, and embodiments of oligonucleotide probe binding to the amplicon at different probe target sequences of the amplicon, according to embodiments of the invention provided herein.

FIGS. 20A-20B are diagrams of an amplicon generated via LAMP amplification with primer set-80 directed to NG rsmB target nucleic acid, and embodiments of oligonucleotide probe binding to the amplicon at different probe target sequences of the amplicon, according to embodiments of the invention provided herein.

FIG. 21 is a diagram of an amplicon generated via LAMP amplification with primer set-81 directed to NG rsmB target nucleic acid, and embodiments of oligonucleotide probe binding to the amplicon at a probe target sequence of the amplicon, according to an embodiment of the invention provided herein.

FIG. 22 is a diagram of an amplicon generated via LAMP amplification with primer set-82 directed to NG rsmB target nucleic acid, and embodiments of oligonucleotide probe binding to the amplicon at a probe target sequence of the amplicon, according to an embodiment of the invention provided herein.

FIG. 23 is a diagram of an amplicon generated via LAMP amplification with primer set-91 directed to NG rsmB target nucleic acid, and embodiments of oligonucleotide probe binding to the amplicon at a probe target sequence of the amplicon, according to an embodiment of the invention provided herein.

FIG. 24 is a diagram of an amplicon generated via LAMP amplification with primer set-83 directed to NG rsmB target nucleic acid, and embodiments of oligonucleotide probe binding to the amplicon at a probe target sequence of the amplicon, according to an embodiment of the invention provided herein.

FIG. 25 is a diagram of an amplicon generated via LAMP amplification with primer set-84 directed to NG rsmB target nucleic acid, and embodiments of oligonucleotide probe binding to the amplicon at a probe target sequence of the amplicon, according to an embodiment of the invention provided herein.

FIGS. 26A-26D are diagrams of an amplicon generated via LAMP amplification with primer set-85 directed to NG rsmB target nucleic acid, and embodiments of oligonucleotide probe binding to the amplicon at different probe target sequences of the amplicon, according to embodiments of the invention provided herein.

FIG. 27 is a diagram of an amplicon generated via LAMP amplification with primer set-57 directed to NG rpIF target nucleic acid, and embodiments of oligonucleotide probe binding to the amplicon at a probe target sequence of the amplicon, according to an embodiment of the invention provided herein.

FIG. 28 is a diagram of an amplicon generated via LAMP amplification with primer set-58 directed to NG rpIF target nucleic acid, and embodiments of oligonucleotide probe binding to the amplicon at a probe target sequence of the amplicon, according to an embodiment of the invention provided herein.

FIG. 29 is a diagram of an amplicon generated via LAMP amplification with primer set-59 directed to NG rpIF target nucleic acid, and embodiments of oligonucleotide probe binding to the amplicon at a probe target sequence of the amplicon, according to an embodiment of the invention provided herein.

FIG. 30 is a diagram of an amplicon generated via LAMP amplification with primer set-60 directed to NG rpIF target nucleic acid, and embodiments of oligonucleotide probe binding to the amplicon at a probe target sequence of the amplicon, according to an embodiment of the invention provided herein.

FIG. 31 is a diagram of an amplicon generated via LAMP amplification with primer set-61 directed to NG rpIF target nucleic acid, and embodiments of oligonucleotide probe binding to the amplicon at a probe target sequence of the amplicon, according to an embodiment of the invention provided herein.

FIGS. 32A-32B are diagrams of an amplicon generated via LAMP amplification with primer set-62 directed to NG rpIF target nucleic acid, and embodiments of oligonucleotide probe binding to the amplicon at different probe target sequences of the amplicon, according to embodiments of the invention provided herein.

### DETAILED DESCRIPTION

The details of various embodiments of the invention are set forth in the description below. Other features, objects, and advantages of the invention will be apparent from the description and the drawings, and from the claims.

Detecting low concentrations of species (down to a few molecules or microorganisms in a sample) is a challenge in research, and especially in diagnostic medicine. The present invention relates to the rapid, selective detection of nucleic acids. In particular, based on new detection strategies utilizing nucleic acid amplification, particularly RT-LAMP, and molecular beacon detection, low concentrations of nucleic acids can be rapidly detected using the methods and reagents described herein. Using RNA (e.g., ribosomal RNA (rRNA) or messenger RNA) as the target regions provides multiple copies of the target nucleic per host genome. Additionally, molecular beacon detection reagents described herein provide additional specificity, failing to bind, in most cases, to off target amplified DNA, thereby minimizing the occurrence of, e.g., false positives. Many other features of the invention are also described herein.

### Definitions

All scientific and technical terms used in this application have meanings commonly used in the art unless otherwise specified. As used in this application, the following words or phrases have the meanings specified.

As used herein, “nucleic acid” includes both DNA and RNA, including DNA and RNA containing non-standard nucleotides. A “nucleic acid” contains at least one polynucleotide (a “nucleic acid strand”). A “nucleic acid” may be single-stranded or double-stranded. The term “nucleic acid” refers to nucleotides and nucleosides which make up, for example, deoxyribonucleic acid (DNA) macromolecules and ribonucleic acid (RNA) macromolecules. Nucleic acids may be identified by the base attached to the sugar (e.g., deoxyribose or ribose).

A “target sequence” or a “target nucleic acid,” as used herein, refers to a nucleic acid sequence of interest, or complement thereof, that, if present in a test sample, is amplified, detected, or both amplified and detected using one or more of the oligonucleotides (e.g., LAMP primers and oligonucleotide probes) provided herein. Additionally, while the term target sequence sometimes refers to a double stranded nucleic acid sequence, those skilled in the art will recognize that the target sequence can also be single stranded, e.g., RNA. A target sequence may be selected that is more or less specific for a particular organism. For example, the target sequence may be specific to an entire genus, to more than one genus, to a species or subspecies, serogroup, auxotype, serotype, strain, isolate or other subset of organisms. In some embodiments, the invention comprises LAMP primers and probes that bind specifically to the target nucleic acid or an amplicon generated using LAMP amplification.

As used herein, a “polynucleotide” or “oligonucleotide” refers to a polymeric chain containing two or more nucleotides, which contain deoxyribonucleotides, ribonucleotides, and/or their analog, such as those containing modified backbones (e.g. peptide nucleic acids (PNAs) or phosphorothioates) or modified bases. Polynucleotides or oligonucleotides include primers, nucleic acid strands, etc. A polynucleotide or oligonucleotide may contain standard or non-standard nucleotides. Thus the term includes mRNA, tRNA, rRNA, ribozymes, DNA, cDNA, recombinant nucleic acids, branched nucleic acids, plasmids, vectors, probes, primers, etc. Typically, a polynucleotide or oligonucleotide contains a 5′ phosphate at one terminus (“5′ terminus”) and a 3′ hydroxyl group at the other terminus (“3′ terminus”) of the chain. The most 5′ nucleotide of an oligonucleotide may be referred to herein as the “5′ terminal nucleotide” of the oligonucleotide. The most 3′ nucleotide of an oligonucleotide may be referred to herein as the “3′ terminal nucleotide” of the oligonucleotide. Where nucleic acid of the invention takes the form of RNA, it may or may not have a 5′ cap.

The term “primer” as used herein refers to an oligonucleotide, which is capable of acting as a point of initiation of synthesis when placed under conditions in which synthesis of primer extension product which is complementary to a nucleic acid strand (template) is induced, i.e., in the presence of nucleotides and an agent for polymerization, such as DNA polymerase, and at a suitable temperature and pH.

As used herein, the term “amplicon” refers to an amplification product from a nucleic acid amplification reaction, e.g., an amplification product generated from a target nucleic acid in the presence of LAMP primers under conditions for LAMP amplification.

As used herein, “oligonucleotide probe” refers to an oligonucleotide having a nucleotide sequence sufficiently complementary to a probe target sequence on an amplicon to be able to form a detectable hybrid probe:target duplex via hybridization. An oligonucleotide probe is an isolated chemical species and may include additional nucleotides outside of the targeted region as long as such nucleotides do not prevent. Non-complementary sequences, such as promoter sequences, restriction endonuclease recognition sites, or sequences that confer a desired secondary or tertiary structure such as a catalytic active site can be used to facilitate detection using the invented probes. An oligonucleotide probe optionally may be labeled with a detectable label such as a radioisotope, a fluorescent moiety, a chemiluminescent moiety, an enzyme or a ligand, which can be used to detect or confirm probe hybridization to its target sequence. “Probe specificity” refers to the ability of a probe to distinguish between target and non-target sequences.

The term “label” or “detectable label” as used herein means a molecule or moiety having a property or characteristic which is capable of detection and, optionally, of quantitation. A label can be directly detectable, as with, for example (and without limitation), radioisotopes, fluorophores, chemiluminophores, enzymes, colloidal particles, fluorescent microparticles and the like; or a label may be indirectly detectable, as with, for example, specific binding members. It will be understood that directly detectable labels may require additional components such as, for example, substrates, triggering reagents, quenching moieties, light, and the like to enable detection and/or quantitation of the label. When indirectly detectable labels are used, they are typically used in combination with a “conjugate”. A conjugate is typically a specific binding member that has been attached or coupled to a directly detectable label. Coupling chemistries for synthesizing a conjugate are well known in the art and can include, for example, any chemical means and/or physical means that does not destroy the specific binding property of the specific binding member or the detectable property of the label. As used herein, “specific binding member” means a member of a binding pair, i.e., two different molecules where one of the molecules through, for example, chemical or physical means specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors or substrates and enzymes; and the like.

The term “quencher” as used herein, refers to a molecule or part of a compound that is capable of reducing light emission (e.g. fluorescence emission) from a detectable label. Quenching may occur by any of several mechanisms, including resonance energy transfer (RET), fluorescence resonance energy transfer (FRET), photo-induced electron transfer, paramagnetic enhancement of intersystem crossing, Dexter exchange coupling, dark quenching, and excitation coupling (e.g., the formation of dark complexes).

As used herein, “molecular beacon” refers to a single stranded hairpin-shaped oligonucleotide probe designed to report the presence of specific nucleic acids in a solution. A molecular beacon consists of four components; a stem, hairpin loop, end labelled fluorophore and opposite end-labelled quencher (Tyagi et al., (1998) Nature Biotechnology 16:49-53). When the hairpin-like beacon is not bound to a target, the fluorophore and quencher lie close together and fluorescence is suppressed. In the presence of a complementary target nucleotide sequence, the stem of the beacon opens to hybridize to the target. This separates the fluorophore and quencher, allowing the fluorophore to fluoresce. Alternatively, molecular beacons also include fluorophores that emit in the proximity of an end-labelled donor. “Wavelength-shifting Molecular Beacons” incorporate an additional harvester fluorophore enabling the fluorophore to emit more strongly. Current reviews of molecular beacons include Wang et al., 2009, Angew Chem Int Ed Engl, 48(5):856-870; Cissell et al., 2009, Anal Bioanal Chem 393(1):125-35; Li et al., 2008, Biochem Biophys Res Comm 373(4):457-61; and Cady, 2009, Methods Mol Biol 554:367-79.

The term “test sample” as used herein, means a sample taken from an organism or biological fluid that is suspected of containing or potentially contains a target sequence. The test sample can be taken from any biological source, such as for example, tissue, blood, saliva, sputa, mucus, sweat, urine, urethral swabs, cervical swabs, vaginal swabs, urogenital or anal swabs, conjunctival swabs, ocular lens fluid, cerebral spinal fluid, milk, ascites fluid, synovial fluid, peritoneal fluid, amniotic fluid, fermentation broths, cell cultures, chemical reaction mixtures and the like. The test sample can be used (i) directly as obtained from the source or (ii) following a pre-treatment to modify the character of the sample. Thus, the test sample can be pre-treated prior to use by, for example, preparing plasma or serum from blood, disrupting cells or viral particles, preparing liquids from solid materials, diluting viscous fluids, filtering liquids, distilling liquids, concentrating liquids, inactivating interfering components, adding reagents, purifying nucleic acids, and the like.

As used herein, the term “specific binding” or “binds specifically to” refers to the targeted binding of an oligonucleotide probe or a primer to a complementary or substantially complementary sequence on a target nucleic acid or an amplicon thereof via hydrogen bonding, i.e., hybridization under. Hybridization is the process by which two complementary or substantially complementary strands of nucleic acid combine to form a double-stranded structure (“hybrid” or “duplex”). The amount of complementarity needed between two nucleic acid strands to form a hybrid can vary based on the temperature and solvent compositions existing during hybridization. Thus, in some embodiments, specific binding referes to the targeted binding of an oligonucleotide probe or a primer to a complementary or substantially complementary sequence on a target nucleic acid or amplicon under LAMP assay conditions. Specifically, in some embodiments, “specific binding” or “binds specifically to” refers to the preferential hybridization of an oligonucleotide probe or primer to its target nucleic acid or target amplicon, under amplification reaction conditions, to form stable primer/probe:target hybrids without forming stable primer/probe:non-target hybrids (that would indicate the presence of non-target nucleic acids in the test sample). Thus, the oligonucleotide hybridizes to its target nucleic acid or target amplicon to a sufficiently greater extent than to non-target nucleic acids to enable one skilled in the art to accurately detect the presence or absence of the relevant target nucleic acid in the test sample. Preferential hybridization can be measured using techniques known in the art.

As used herein, “complementarity” is a property conferred by the base sequence of a single strand of DNA or RNA which may form a hybrid or double-stranded DNA:DNA, RNA:RNA or DNA:RNA through hydrogen bonding between Watson-Crick base pairs on the respective strands. Adenine (A) ordinarily complements thymine (T) or Uracil (U), while guanine (G) ordinarily complements cytosine (C). “Fully complementary”, when describing a probe with respect to its target sequence, means that complementarity is present along the full length of the probe.

One skilled in the art will understand that substantially complementary probes of the invention can vary from the referred-to sequence and still hybridize preferentially to the same target nucleic acid sequence. This variation from the nucleic acid may be stated in terms of a percentage of identical bases within the sequence or the percentage of perfectly complementary bases between the probe and its target sequence. Probes of the present invention are substantially identical to a nucleic acid sequence if these percentages are from 100% to 80% or from 0 base mismatches in a 10 nucleotide target sequence to 2 bases mismatched in a 10 nucleotide target sequence. In preferred embodiments, the percentage is from 100% to 85%. In more preferred embodiments, this percentage is from 90% to 100%; in other preferred embodiments, this percentage is from 95% to 100%.

In the context of LAMP amplification and/or detection, “substantially identical” oligonucleotides have a sufficient amount of contiguous complementary nucleotides to preferentially hybridize or bind specifically to the same complementary sequence under amplification reaction conditions.

In the context of LAMP amplification and/or detection, “substantially complementary” oligonucleotides refer to oligonucleotides having a sufficient amount of contiguous complementary nucleotides to preferentially form a hybrid with a target nucleic acid or target amplicon under amplification reaction conditions. When primers are substantially complementary to a target sequence, they are sufficiently complementary to form a hybrid to further LAMP amplification via template-directed synthesis based on the complementary target sequence from the 3′ end of the primer. In the context of oligonucleotide probe binding, substantially complementary refers to an oligonucleotide probe having a sufficient amount of contiguous complementary nucleotides to preferentially hybridize with a sequence on a target nucleic acid or amplicon under assay conditions (e.g., LAMP assay conditions) to facilitate detection of the presence or absence of the target nucleic acid or amplicon, e.g. during or after a LAMP amplification reaction.

### Overview

LAMP is a nucleic acid amplification method that relies on auto-cycle strand-displacement DNA synthesis performed by a Bst DNA polymerase, or other strand displacement polymerases. The amplified products are stem-loop structures with several repeated sequences of the target, and have multiple loops. The principal merit of this method is that denaturation of the DNA template is not required, and thus the LAMP reaction can be conducted under isothermal conditions (ranging from 60 to 67° C.) LAMP typically requires only one enzyme and four types of primers that recognize six distinct hybridization sites in the target sequence (inner and outer primers). The reaction can be accelerated by the addition of two additional primers (loop primers). The method produces a large amount of amplified product, resulting in easier detection, such as detection by visual judgment of the turbidity or fluorescence of the reaction mixture.

In brief, the reaction is initiated by annealing and extension of a pair of ‘loop-forming’ primers (forward and backward inner primers, FIP and BIP, respectively). In some embodiments, this is followed by annealing and extension of a pair of flanking primers (F3 and B3).

Extension of these primers results in strand-displacement of the loop-forming elements, which fold up to form terminal hairpin-loop structures. Once these key structures have appeared, the amplification process becomes self-sustaining, and proceeds at constant temperature in a continuous and exponential manner (rather than a cyclic manner, like PCR) until all of the nucleotides (dATP, dTTP, dCTP & dGTP) in the reaction mixture have been incorporated into the amplified DNA. Optionally, an additional pair of primers can be included to accelerate the reaction. These primers, termed Loop primers, hybridize to non-inner primer bound terminal loops of the inner primer dumbbell shaped products.

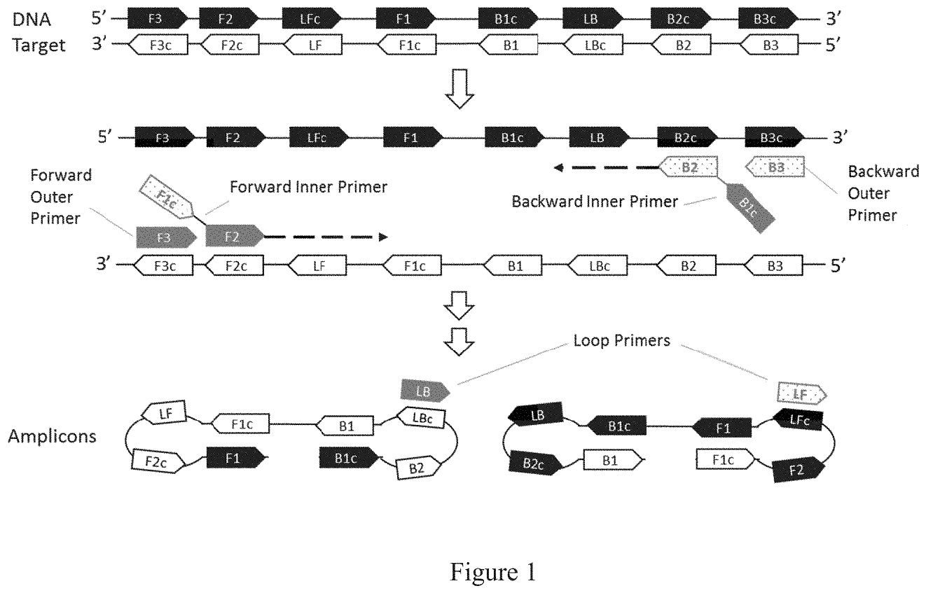

Shown in FIG. 1 is a diagram showing binding of primers that can be used in LAMP to target sequences and amplicons. Specifically FIG. 1 shows a nucleic acid target (DNA). A first strand, designated as the sense strand, includes regions that are related to their sequence identity or sequence complementarity to the primer sequences used in the LAMP reaction. The F1, F2, B1, and B2 regions comprises sequences substantially complementary to or substantially identical to sequences in the inner primers (i.e., Forward Inner Primer (FIP) and Backward Inner Primer (BIP). Regions ending with a “c,” such as F1c, F2c, B1c, B2c are identified as regions with a sequence complementary to the region without a C, i.e., F1c has a sequence complementary to F1. The F3c and B3c regions comprise sequences substantially complementary to outer primers F3 and B3. The LFc and LBc regions of the target nucleic acid comprise sequences substantially complementary to the loop primers LF and LB, respectively.

To initiate a LAMP reaction, an inner primer binds to a complementary sequence on the target nucleic acid. A forward inner primer (FIP) or a backward inner primer (BIP) can initiate generation of an amplicon from the target nucleic acid, each of which generate a distinct seed amplicon.

In some embodiments, a forward inner primer (FIP) initiates template directed synthesis from a target nucleic acid to generate an amplicon. First, an F2 region of the FIP hybridizes to a substantially complementary region F2c on the target nucleic acid (FIG. 1). A strand displacement polymerase then catalyzes template-dependent polymerization to generate a polynucleotide strand with a 5′ end containing, in the 5′ to 3′ direction, an F1c region, an F2 region, and a F1 region. The synthesized strand can be displaced by using a forward outer primer, F3, which has a sequence substantially complementary with the F3c region of the target nucleic acid to allow specific hybridization. Elongation of the F3 primer by a strand displacement polymerase induces displacement of the strand synthesized from the forward inner primer. In a subsequent step, a backwards inner primer, binds to the displaced strand to catalyze template-dependent polymerization to generate a polynucleotide strand with a 5′ end containing, in the 5′ to 3′ direction, a B1c region, a B2 region, and a B1 region, and a 3′ end containing, in the to 3′ to 5′ direction, an F1 region, an F2c region, and an F1c region. This strand can be displaced from the template using a B3 primer and a strand displacement polymerase, forming a seed amplicon that includes two loop regions and a central region between the loops (FIG. 1). The loops are formed due to the presence of the substantially complementary F1c and F1 regions on the 3′ end and the substantially complementary B1c and B1 regions on the 5′ end.

In some embodiments, a backward inner primer (BIP) initiates template-directed synthesis from a target nucleic acid to generate an amplicon. First, a B2 region of the BIP hybridizes to a substantially complementary region B2c on the target nucleic acid (FIG. 1). A strand displacement polymerase then catalyzes template-dependent polymerization to generate a polynucleotide strand with a 5′ end containing, in the 5′ to 3′ direction, a B1c region, a B2 region, and a B1 region. As described in the previous paragraph, a B3 primer can then be used to displace the newly synthesized strand, followed by subsequent template directed polymerization from hybridization of the FIP to the displaced strand. Displacement of the newly synthesized strand using an F3 primer results in the formation of a seed amplicon that includes two loops and a central region between the loops.

The two different types of seed amplicons formed are shown in FIG. 1. Each amplicon includes a “DS” region, which is defined herein the portion of the amplicon extending from the 5′ end of the B1 region to the 3′ end of the F1c region, or as the portion of the amplicon extending from the 5′ end of the F1 region to the 3′ end of the B1c region. The portion of the amplicons shown in FIG. 1 outside of the DS regions are referred to herein as the “loop” regions. Numerous amplicons of varying structure can be generated via LAMP, having a stem (double-stranded) and loop (single-stranded) structure. The loops in these amplicons are outside of the DS regions, and thus do not include the portion of the amplicon defined by the B1-F1c or F1-B1c regions. Since the loops comprise a single-stranded polynucleotide, their sequences are accessible for binding to other oligonucleotides, such as inner primers, loop primers, and oligonucleotide probes. As such, the DS regions of amplicons have not been used as probe target sequences for oligonucleotide probes.

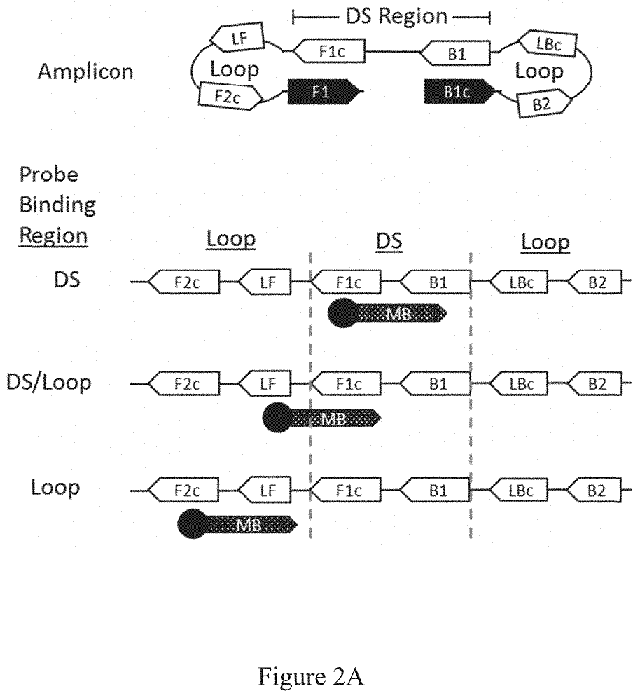

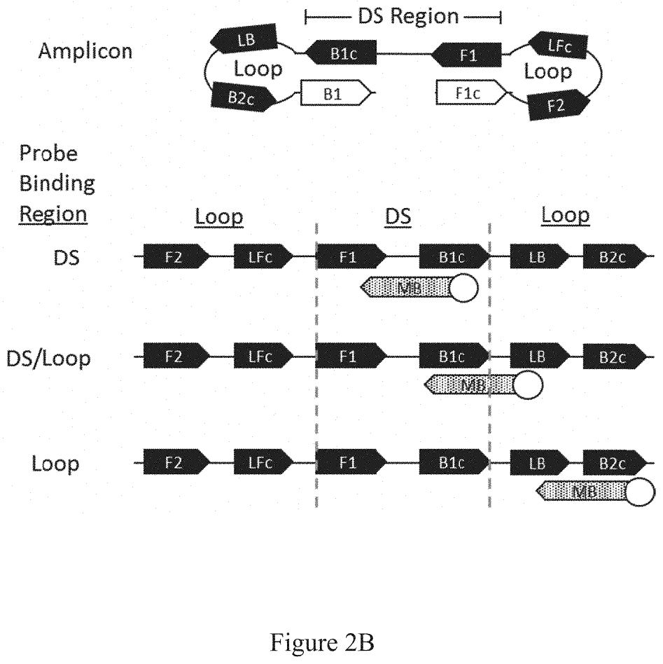

Provided herein, according to some embodiments, are novel oligonucleotide probes for detection of the presence or absence of amplicons generated from a target sequence via LAMP. In some embodiments, the oligonucleotide probes bind specifically to a portion of the amplicon (i.e., the probe target sequence) that is within or overlaps a DS region of an amplicon. FIG. 2A and 2B shows embodiments of probe binding to regions of a LAMP amplicon. FIG. 2A shows one type of seed amplicon comprising a DS region extending from the 5′ end of the B1 region to the 3′ end of the F1c region (i.e., a first region). FIG. 2B shows another type of seed amplicon comprising a DS region extending from the 5′ end of the F1 region to the 3′ end of the B1c region (i.e., a second region). Frequently, LAMP amplicons will comprise both complementary strands of the DS region (i.e., the first region and second region) forming a double-stranded portion of an amplicon. Throughout the specification, embodiments of oligonucleotides that bind within the DS region (DS), to a portion of the DS region and a portion of the loop region (DS/Loop), or completely outside of the DS region (Loop) are provided and tested. Exemplary embodiments of each type of oligonucleotide probe binding are shown in FIG. 2A and FIG. 2B.

Regions of the target nucleic acid, amplicons, and primers are discussed in the context of regions of these nucleic acids that are related to primers that can be used in LAMP, i.e., F1, F1c, F2, F2c, F3, F3c, B1, B1c, B2, B2c, B3, B3c, LF, LFc, LB, and LBc. As used herein, these regions are defined by their relationship along the length of a target nucleic acid, an amplicon, or a primer, and are based on primer-target nucleic acid hybridization, primer-amplicon hybridization, or loop structure formation in an amplicon via hybridization of F1 and F1c or B1 and B1c. The structure of the amplicon can also be described with respect to these regions.

As used herein, regions that are labeled as the same in a target nucleic acid, amplicon, and primer have a substantially identical nucleotide sequence. Regions that are labeled ending in a ‘c’ are substantially complementary to their counterpart regions that do not end in a ‘c’ (i.e., F1c and F1). Sequence variability between the same regions on different types of molecules (i.e., target nucleic acids, amplicons, and primers) can occur due to the presence of one or more mismatches along the length of the complementary region. If the sequences are substantially complementary, then hybridization will still occur to generate LAMP amplicons from a target nucleic acid, while the sequence of the same region on the primer may vary from the target nucleic acid, and thus certain sequences incorporated from the primer will vary in the amplicon as compared to the target nucleic acid. For example, an F1c region of a forward inner primer may vary by one or more polynucleotides but still hybridize to an F1 region of a target nucleic acid to allow generation of a complementary strand subsequent to the F1c region. After displacement, a backward inner primer can initiate template directed synthesis from the displaced strand to generate a seed amplicon, such that the seed amplicon has B1c and B2 regions that have the sequence of the primer, F1 and F2c regions that are complementary to the outer primer, such that the F2c region has a substantially identical, but not 100% identical sequence to the F2c region of the target nucleic acid. This level of flexibility is provided as specific binding via hybridization to induce LAMP amplification can occur without a perfect sequence complement.

In some embodiments, the regions of the target nucleic acid are spaced close together to generate a shorter amplicon. In some embodiments, the amplicon is generated from a portion of the target nucleic acid including and extending from the F2 region to the B2c region or the F2c region to the B2 region that is less than 200 base pairs. In some embodiments, this portion of the target nucleic acid is less than 200 bp, less than 190 bp, less than 180 bp, less than 170 bp, less than 160 bp, less than 150 bp, less than 140 bp, less than 130 bp, or less than 120 bp in length as measured from the F2 region to the B2c region (or the F2c region to the B2 region) of a target nucleic acid. In some embodiments, the largest space between two primer binding regions is no more than 1 bp, no more than 2 bp, no more than 3 bp, no more than 4 bp, no more than 5 bp, no more than 6 bp, no more than 7 bp, no more than 8 bp, no more than 9 bp, or no more than 10 bp.

LAMP allows amplification of target DNA sequences with higher sensitivity and specificity than PCR, often with reaction times of below 30 minutes, which is equivalent to or better than the fastest real-time PCR tests. The target sequence which is amplified is conventionally 200-300 base-pairs (bp) in length, and the reaction relies upon recognition of between 120 bp and 160 bp of this sequence by several primers simultaneously during the amplification process. The present disclosure demonstrates that the target sequence can be as short as approximately 119 bp (measured from the F2 region to the B2 region), of which nearly all bases in the target sequence are recognized by a primer during the amplification process. This high level of complementarity makes the amplification highly specific, such that the appearance of amplified DNA in a reaction occurs only if the entire target sequence was initially present.

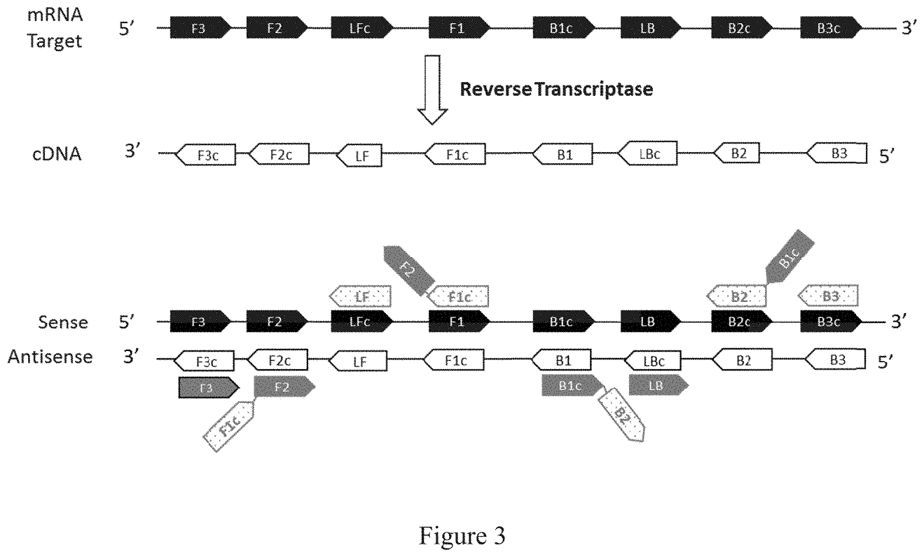

Applications for LAMP have been further extended to include detection of RNA molecules by addition of Reverse Transcriptase enzyme (RT) (FIG. 3). By including RNA detection, the types of targets for which LAMP can be applied are also expanded and add the ability to additionally target RNA based viruses, important regulatory non-coding RNA (sRNA, miRNA), and RNA molecules that have been associated with particular disease or physiological states. The ability to detect RNA also has the potential to increase assay sensitivity, for instance in choosing highly expressed, stable, and/or abundant messenger RNA (mRNA) or ribosomal RNA (rRNA) targets. This preliminary phase of amplification involves the reverse transcription of RNA molecules to complementary DNA (cDNA). The cDNA then serves as template for the strand displacing DNA polymerase. Use of a thermostable RT enzyme (i.e. NEB RTx) enables the reaction to be completed at a single temperature and in a one step, single mix reaction.

As shown in FIG. 3, a sense strand of mRNA can be converted to an antisense nucleic acid template for subsequent LAMP amplification. The sense strand regions are shown in black, and the antisense strand regions are shown in white. The regions are defined by the LAMP primer binding, as shown in FIG. 3 and described above.

### LAMP Primers

The speed, specificity and sensitivity of the primers/probe compositions and method described herein result from several aspects. Exemplary primers for use in the compositions and methods according to the present invention are provided in Table 1.

TABLE 1
Primer Sequences

| Organism | Target | Primer ID | SEQ ID NO: | Sequence (5′ to 3′) |
|---|---|---|---|---|
| CT | 23S | 23S-F3, s6 | SEQ ID NO: 1 | CTGAAACATCTTAGTAAGCAGAGG |
| CT | 23S | 23S-B3, s6 | SEQ ID NO: 2 | GTGTCTAGTCCTACTCAGGTG |
| CT | 23S | 23S-FIP, s6 | SEQ ID NO: 3 | CCTACAACCCCGAGCCTTATCAAGAGATTCCCTGTGT |
| | | | | AGCG |
| CT | 23S | 23S-BIP, s6 | SEQ ID NO: 4 | GGACTCCTAGTTGAACACATCTGGATTCTCTCCTTTC |
| | | | | GTCTACGG |
| CT | 23S | 23S-LF, S1 | SEQ ID NO: 5 | GCTCGGTTTAGGCTATTCCC |
| CT | 23S | 23S-LB, s6 | SEQ ID NO: 6 | AAGATGGATGATACAGGGTGATAGT |
| CT | 23S | 23S-FIP, s7bis | SEQ ID NO: 7 | ATCCTTTATCCTCAATCCTACAACCGTAGCGGCGAGC |
| | | | | GAAAG |
| CT | 23S | 23S-LF, s7 | SEQ ID NO: 8 | AGCCTTATCAGCTCGGTT |
| CT | 23S | 23S-F3, s8 | SEQ ID NO: 9 | GACACCTGCCGAACTG |
| CT | 23S | 23S-B3, s8 | SEQ ID NO: 10 | AGCCTTGGAGAGTGGT |
| CT | 23S | 23S-FIP, s8 | SEQ ID NO: 11 | TCCTGATCCTTTATCCTCAATCCTACGAGATTCCCTGT |
| | | | | GTAGCG |
| CT | 23S | 23S-BIP, s8 | SEQ ID NO: 12 | TCCTAGTTGAACACATCTGGAAAGAAGGTGTTGAGG |
| | | | | TCGGT |
| CT | 23S | 23S-LB, s8 | SEQ ID NO: 13 | CCGTAGACGAAAGGAGAGAA |
| CT | 23S | 23S-F3, s11 | SEQ ID NO: 14 | CCTGCCGAACTGAAACATC |
| CT | 23S | 23S-B3, s11 | SEQ ID NO: 15 | GGTGTTGAGGTCGGTCTT |
| CT | 23S | 23S-FIP, s11 | SEQ ID NO: 16 | CCTACAACCCCGAGCCTTATCAAGAGATTCCCTGTGT |
| | | | | AGCG |
| CT | 23S | 23S-BIP, s11 | SEQ ID NO: 17 | GGATCAGGACTCCTAGTTGAACACACTTTCGTCTACG |
| | | | | GGACTATCA |
| CT | 23S | 23S-LB, 511 | SEQ ID NO: 18 | CTGGAAAGATGGATGATACAGGG |
| CT | 23S | 23S-F3, s12 | SEQ ID NO: 19 | GGGTTGTAGGATTGAGGATAAAGG |
| CT | 23S | 23S-B3, s12 | SEQ ID NO: 20 | GGTTCACTATCGGTCATTGACTAG |
| CT | 23S | 23S-FIP, s12 | SEQ ID NO: 21 | TTTCTCTCCTTTCGTCTACGGGACTATCAGGACTCCTA |
| | | | | GTTGAACACA |
| CT | 23S | 23S-BIP, s12 | SEQ ID NO: 22 | ACCGACCTCAACACCTGAGTAGGTTAGCCTTGGAGA |
| | | | | GTGGTCTC |
| CT | 23S | 23S-LF, s12 | SEQ ID NO: 23 | CACCCTGTATCATCCATCTTTCCAG |
| CT | 23S | 23S-LB, s12 | SEQ ID NO: 24 | CGTGAAACCTAGTCTGAATCTGGG |
| CT | 23S | 23S-F3, s13 | SEQ ID NO: 25 | GTAGGATTGAGGATAAAGG |
| CT | 23S | 23S-B3, s13 | SEQ ID NO: 26 | CAGTACTGGTTCACTATC |
| CT | 23S | 23S-FIP, s13 | SEQ ID NO: 27 | TCTTTCTCTCCTTTCGTCTACCTAGTTGAACACATCTG |
| | | | | G |
| CT | 23S | 23S-BIP, s13 | SEQ ID NO: 28 | AACACCTGAGTAGGACTAGACTAGTATTTAGCCTTG |
| | | | | GAG |
| CT | 23S | 23S-LF, s13 | SEQ ID NO: 29 | CCCTGTATCATCCATCTTT |
| CT | 23S | 23S-LB, s13 | SEQ ID NO: 30 | TGAAACCTAGTCTGAATCTG |
| CT | 23S | Ctr23S-F3, n2 | SEQ ID NO: 31 | CGTAACAGCTCACCAATCG |
| CT | 23S | Ctr23S-B3, n2 | SEQ ID NO: 32 | TACGCAGTTACGCCTCAA |
| CT | 23S | Ctr23S-FIP, | SEQ ID NO: 33 | CGCTCCTTCCGGTACACCTTTCGATAAGACACGCGGT |
| | | n2 | | AG |
| CT | 23S | Ctr23S-BIP, | SEQ ID NO: 34 | AATCTCCCTCGCCGTAAGCCGACTAACCCAGGGAAG |
| | | n2 | | ACG |
| CT | 23S | Ctr23S-LF, n2 | SEQ ID NO: 35 | CTCTGCTGAATACTACGCTCTC |
| CT | 23S | Ctr23S-LB, n2 | SEQ ID NO: 36 | CAAGGTTTCCAGGGTCAAGC |
| CT | 23S | 23S-F3, n3 | SEQ ID NO: 37 | CCAAGGTTTCCAGGGTCAA |
| CT | 23S | 23S-B3, n3 | SEQ ID NO: 38 | CCGAAGATTCCCCTTGATCG |
| CT | 23S | 23S FIP, n3 | SEQ ID NO: 39 | CTGCTCCATCGTCTACGCAGTTTGCTCGTCTTCCCTG |
| | | | | GGTT |
| CT | 23S | 23S BIP, n3 | SEQ ID NO: 40 | ACGGAGTAAGTTAAGCACGCGGTGCGGATTTGCCTA |
| | | | | CTAACCG |
| CT | 23S | 23S-LF, n3 | SEQ ID NO: 41 | CTCAACTTAGGGGCCGACT |
| CT | 23S | 23S-LB, n3 | SEQ ID NO: 42 | ACGATTGGAAGAGTCCGTAGAG |
| CT | 23S | 23S-F3, S1 | SEQ ID NO: 43 | TATGCAAAGCGACACCTG |
| CT | 23S | 23S-B3, S1 | SEQ ID NO: 44 | TTAGCCTTGGAGAGTGGTC |
| CT | 23S | 23S FIP, s1 | SEQ ID NO: 45 | TCCTCAATCCTACAACCCCGAGCGAAGAGATTCCCTG |
| | | | | TGTAG |
| CT | 23S | 23S BIP, s1 | SEQ ID NO: 46 | GGGTGATAGTCCCGTAGACGAACGTGTCTAGTCCTA |
| | | | | CTCAGG |
| CT | 23S | 23S-LB, s1 | SEQ ID NO: 47 | GAGAGAAAGACCGACCTCAAC |
| CT | 23S | 23S-F3, s2(1) | SEQ ID NO: 48 | AGAGATTCCCTGTGTAGCG |
| CT | 23S | 23S-B3, s2(1) | SEQ ID NO: 49 | CCTTCACAGTACTGGTTCAC |
| CT | 23S | 23S-FIP, s2(1) | SEQ ID NO: 50 | CTCTCCTTTCGTCTACGGGACTAACCGAGCTGATAAG |
| | | | | GCT |
| CT | 23S | 23S-BIP, s2(1) | SEQ ID NO: 51 | AAGACCGACCTCAACACCTGATTAGCCTTGGAGAGT |
| | | | | GGTC |
| CT | 23S | 23S-LF, s2(1) | SEQ ID NO: 52 | GTTCAACTAGGAGTCCTGATCC |
| CT | 23S | 23S-LB, s2(1) | SEQ ID NO: 53 | GTAGGACTAGACACGTGAAACC |
| CT | 23S | 23S-F3, s7 | SEQ ID NO: 54 | CATGCTGAATACATAGGTATGC |
| CT | 23S | 23S-B3, s7 | SEQ ID NO: 55 | TCTAGTCCTACTCAGGTGTT |
| CT | 23S | 23S FIP, s7 | SEQ ID NO: 56 | TCCTTTATCCTCAATCCTACAACCCATCGAAGAGATTC |
| | | | | CCTGTG |
| CT | 23S | 23S BIP, s7 | SEQ ID NO: 57 | ACTCCTAGTTGAACACATCTGGAATCTTTCTCTCCTTT |
| | | | | CGTCTAC |
| CT | 23S | 23S-LB, s7 | SEQ ID NO: 58 | TGGATGATACAGGGTGATAGTC |
| CT | 23S | 23S-F3, s12 | SEQ ID NO: 59 | GGGTTGTAGGATTGAGGATAAAGG |
| CT | 23S | 23S-B3, s12 | SEQ ID NO: 60 | GGTTCACTATCGGTCATTGACTAG |
| CT | 23S | 23S-FIP, s12 | SEQ ID NO: 61 | TTTCTCTCCTTTCGTCTACGGGACTATCAGGACTCCTA |
| | | | | GTTGAACACA |
| CT | 23S | 23S-BIP, s12 | SEQ ID NO: 62 | ACCGACCTCAACACCTGAGTAGGTTAGCCTTGGAGA |
| | | | | GTGGTCTC |
| CT | 23S | 23S-LF, s12 | SEQ ID NO: 63 | CACCCTGTATCATCCATCTTTCCAG |
| CT | 23S | 23S-LB, s12 | SEQ ID NO: 64 | CGTGAAACCTAGTCTGAATCTGGG |
| CT | 23S | 23S-F3-1, s14 | SEQ ID NO: 65 | CGAACTGAAACATCTTAGTAAGCAG |
| CT | 23S | 23S-B3, s14 | SEQ ID NO: 66 | CTCCTTTCGTCTACGGGACTA |
| CT | 23S | 23S-FIP1, s14 | SEQ ID NO: 67 | ATCAGCTCGGTTTAGGCTATTCCCGAAAAGAAATCG |
| | | | | AAGAGATTCCCTG |
| CT | 23S | 23S-BIP, s14 | SEQ ID NO: 68 | GCTCGGGGTTGTAGGATTGAGGATACCTGTATCATC |
| | | | | CATCTTTCCAGAT |
| CT | 23S | 23S-LF, s14 | SEQ ID NO: 69 | CTTTCGCTCGCCGCTAC |
| CT | 23S | 23S-LB, s14 | SEQ ID NO: 70 | GGATCAGGACTCCTAGTTGAACAC |
| CT | 23S | 23S-F3, n1 | SEQ ID NO: 71 | CTTACAAGCGGTCGGAGA |
| CT | 23S | 23S-B3, n1 | SEQ ID NO: 72 | CAGGTACTAGTTCGGTCCTC |
| CT | 23S | 23S FIP, n1 | SEQ ID NO: 73 | CCCTTAACCTCGCCGTTTAGCCCCGTAAGGGTCAAG |
| | | | | GTT |
| CT | 23S | 23S BIP, n1 | SEQ ID NO: 74 | CCGGAGCGAAAGCGAGTTTGCTCACTTGGTTTCGTG |
| | | | | TC |
| CT | 23S | 23S-LF, n1 | SEQ ID NO: 75 | TCCCTGGCTCATCATGCA |
| CT | 23S | 23S-LB, n1 | SEQ ID NO: 76 | GAGCGAAGAGTCGTTTGGTT |
| CT | 16S | 16S-F3, s1bis | SEQ ID NO: 77 | GGAGCAATTGTTTCGACG |
| CT | 16S | 16S-B3, s1 | SEQ ID NO: 78 | TGTCTCAGTCCCAGTGTT |
| CT | 16S | 16S FIP, s1 | SEQ ID NO: 79 | GCCCAAATATCGCCACATTCGGGCGGAAGGGTTAGT |
| | | | | AATG |
| CT | 16S | 16S BIP, s1 | SEQ ID NO: 80 | GACCTTTCGGTTAAGGGAGAGTCGACGTCATAGCCT |
| | | | | TGGTAG |
| CT | 16S | 16S-LF, s1 | SEQ ID NO: 81 | CGTTTCCAACCGTTATTCCC |
| CT | 16S | 16S-LB, s1 | SEQ ID NO: 82 | AGTTGGTGGGGTAAAGGC |
| CT | 16S | 16S-F3, s6 | SEQ ID NO: 83 | TTAGTGGCGGAAGGGTTAG |
| CT | 16S | 16S-B3, s6 | SEQ ID NO: 84 | TCTCAATCCGCCTAGACG |
| CT | 16S | 16S FIP, s6 | SEQ ID NO: 85 | AACGTTACTCGGATGCCCAAATGGAATAACGGTTGG |
| | | | | AAACGG |
| CT | 16S | 16S BIP, s6 | SEQ ID NO: 86 | AGGACCTTTCGGTTAAGGGAGATAGCCTTGGTAGGC |
| | | | | CTTTAC |
| CT | 16S | 16S-LF, s6 | SEQ ID NO: 87 | ATCGCCACATTCGGTATTAGC |
| CT | 16S | 16S-LB, s6 | SEQ ID NO: 88 | GTGATATCAGCTAGTTGGTGGG |
| CT | 16S | 16S-F3, s7 | SEQ ID NO: 89 | GAACGGAGCAATTGT |
| CT | 16S | 16S-B3, s7 | SEQ ID NO: 90 | CTGATATCACATAGACTCTC |
| CT | 16S | 16S-FIP, s7 | SEQ ID NO: 91 | CCGTTTCCAACCGTTATTCTCGACGATTGTTTAGTG |
| CT | 16S | 16S BIP, s7 | SEQ ID NO: 92 | TACCGAATGTGGCGATATTTCGAAAGGTCCTAAGAT |
| | | | | C |
| CT | 16S | 16S-LF, s7 | SEQ ID NO: 93 | CTATGCATTACTAACCCTTC |
| CT | 16S | 16S-LB, s7 | SEQ ID NO: 94 | CATCCGAGTAACGTTAAAG |
| NG | 23S | 23S-F3-n2 | SEQ ID NO: 95 | CCGGCTAAGGTCCCAAAT |
| NG | 23S | 23S-B3- n2 | SEQ ID NO: 96 | TCGCACTTCTGATACCTCC |
| NG | 23S | 23S-FIP-n2 | SEQ ID NO: 97 | TCGACCAGTGAGCTATTACGCTGAAGTGGGAAGGCA |
| | | | | CAGA |
| NG | 23S | 23S-BIP-n2 | SEQ ID NO: 98 | CTATAACCGAAGCTGCGGATGCATCAGCCTACAGAA |
| | | | | CGCTC |
| NG | 23S | 23S-LF-n2 | SEQ ID NO: 99 | GCTTCTAAGCCAACATCCTGG |
| NG | 23S | 23S-LB-n2 | SEQ ID NO: 100 | CGGTTTACCGGCATGGTAG |
| NG | 23S | 23S-F3-s1 | SEQ ID NO: 101 | AGAGAACTCGGGAGAAGGAA |
| NG | 23S | 23S-B3-S1 | SEQ ID NO: 102 | TCGCTACCTTAGGACCGTTA |
| NG | 23S | 23S-FIP-s1 | SEQ ID NO: 103 | CAGCCACCTATTCTCTGCGACCGGAGAAGGTATGCC |
| | | | | CTCTAAG |
| NG | 23S | 23S-BIP-s1 | SEQ ID NO: 104 | CGTATAGGGTGTAACGCCTGCCGGCTTCGATCCGAT |
| | | | | GCTT |
| NG | 23S | 23S-LF-s1 | SEQ ID NO: 105 | GGCTTACGGAGCAAGTCCT |
| NG | 23S | 23S-LB-s1 | SEQ ID NO: 106 | CGGTGCCGGAAGGTTAATTG |
| NG | 23S | 23S-F3-s2 | SEQ ID NO: 107 | GGAGAAGGAACTCGGCAA |
| NG | 23S | 23S-B3-s2 | SEQ ID NO: 108 | CTTCGATCCGATGCTTGC |
| NG | 23S | 23S-FIP-s2 | SEQ ID NO: 109 | AGCCACCTATTCTCTGCGACCGGAGAAGGTATGCCC |
| | | | | TCTAA |
| NG | 23S | 23S-BIP-s2 | SEQ ID NO: 110 | GAGCACTCTTGCCAACACGAACAATTAACCTTCCGGC |
| | | | | ACC |
| NG | 23S | 23S-LF-s2 | SEQ ID NO: 111 | CTTACGGAGCAAGTCCTTAACC |
| NG | 23S | 23S-LB-s2 | SEQ ID NO: 112 | GTATAGGGTGTAACGCCTGC |
| NG | 23S | 23S-BIP-new2 | SEQ ID NO: 113 | AGCACTCTGCCAACACGAACCACGGCCTTCCAATTAA |
| | | | | C |
| NG | 23S | 23S-LB-new2 | SEQ ID NO: 114 | GTATAGGGTGTCACGCCTGC |
| NG | 23S | 23S-F3-s3 | SEQ ID NO: 115 | AGAGAATAGGTGGCTGCGA |
| NG | 23S | 23S-B3-s3 | SEQ ID NO: 116 | AGACAGTGTGGCCATCGT |
| NG | 23S | 23S-FIP-s3 | SEQ ID NO: 117 | GCAGGCGTCACACCCTATACGGTTTATTAAAAACACA |
| | | | | GCACTCTGCC |
| NG | 23S | 23S-BIP-s3 | SEQ ID NO: 118 | CGGTAAACGGCGGCCGTAAATTCGTGCGGGTCGGA |
| | | | | AC |
| NG | 23S | 23S-LF-s3 | SEQ ID NO: 119 | CTATACGTCCACTTTCGTGTTG |
| NG | 23S | 23S-LB-s3 | SEQ ID NO: 120 | TAACGGTCCTAAGGTAGCGAA |
| NG | 16S | 16S-F3-n1 | SEQ ID NO: 121 | GAGCGCAACCCTTGTCAT |
| NG | 16S | 16S-B3-n1 | SEQ ID NO: 122 | TCCGACTTCATGCACTCGA |
| NG | 16S | 16S-FIP-n1 | SEQ ID NO: 123 | GAGGACTTGACGTCATCCCCACGGGCACTCTAATGA |
| | | | | GACTGC |
| NG | 16S | 16S-BIP-n1 | SEQ ID NO: 124 | ATGGTCGGTACAGAGGGTAGCCAGTGCAATCCGGA |
| | | | | CTACGAT |
| NG | 16S | 16S-LF-n1 | SEQ ID NO: 125 | CTTCCTCCGGCTTGTCACCG |
| NG | 16S | 16S-LB-n1 | SEQ ID NO: 126 | AGGCGGAGCCAATCTCACAAAAC |
| NG | rsmB | rsm-F3-1 | SEQ ID NO: 127 | ATGCCGAAAGCTATTTGG |
| NG | rsmB | rsm-B3-1 | SEQ ID NO: 128 | GCCGTCTTTCGGGTTA |
| NG | rsmB | rsm-FIP-1 | SEQ ID NO: 129 | CTTCTTCCAACGTAACCGCATAGTGGCGGAAGGTAT |
| | | | | CG |
| NG | rsmB | rsm-BIP-1 | SEQ ID NO: 130 | GCCGGTAAACCGTCTGCCCGAAGTCCTGTACCG |
| NG | rsmB | rsm-LF-1 | SEQ ID NO: 131 | CGTCCAACGCCTTAGC |
| NG | rsmB | rsm-LB-1 | SEQ ID NO: 132 | TTGCCGAAGGACTGGT |
| NG | rsmB | rsm-F3-2 | SEQ ID NO: 133 | CTGGTGGCGGAAGGT |
| NG | rsmB | rsm-B3-2 | SEQ ID NO: 134 | ATCCGTTCGCCGTCT |
| NG | rsmB | rsm-FIP-2 | SEQ ID NO: 135 | GGCACGGCTTCTTCCAAATCGCGGCTAAGGC |
| NG | rsmB | rsm-BIP-2 | SEQ ID NO: 136 | TTTGCCGAAGGACTGGTGATACGCCGCCTGC |
| NG | rsmB | rsm-LF-2 | SEQ ID NO: 137 | GTAACCGCATATTCGTCCA |
| NG | rsmB | rsm-LB-2 | SEQ ID NO: 138 | TCGGTACAGGACTTCGG |
| NG | rsmB | rsm-F3-3 | SEQ ID NO: 139 | AAGCTATTTGGAAAAACTGGT |
| NG | rsmB | rsm-FIP-3 | SEQ ID NO: 140 | CTTCTTCCAACGTAACCGCATAGAAGGTATCGCGGCT |
| NG | rsmB | rsm-BIP-3 | SEQ ID NO: 141 | CGTGCCGGTAAACCGTCGCCGAAGTCCTGT |
| NG | rsmB | rsm-LF-3 | SEQ ID NO: 142 | CATATTCGTCCAACGCCTTA |
| NG | rsmB | rsm-LB-3 | SEQ ID NO: 143 | TGCCGAAGGACTGGT |
| NG | rsmB | rsm-F3-4 | SEQ ID NO: 144 | TTGGACGAATATGCGGTT |
| NG | rsmB | rsm-B3-4 | SEQ ID NO: 145 | CCGTCTGAAAGCCCAAA |
| NG | rsmB | rsm-FIP-4 | SEQ ID NO: 146 | AGATACGCCGCCTGCTGCGTTGGAAGAAGCCGTG |
| NG | rsmB | rsm-BIP-4 | SEQ ID NO: 147 | TGGAACTGGCGGACTGCGGCGATATTGTCCTCCAC |
| NG | rsmB | rsm-LF-4 | SEQ ID NO: 148 | CCGAAGTCCTGTACCGAC |
| NG | rsmB | rsm-LB-4 | SEQ ID NO: 149 | CGTTACCGCCTTGGACA |
| NG | rsmB | rsm-F3-5 | SEQ ID NO: 150 | TTGGACGAATATGCGGTTA |
| NG | rsmB | rsm-FIP-5 | SEQ ID NO: 151 | CGCCGTCTTTCGGGTTAAGGAGCCGTGCCGGTA |
| NG | rsmB | rsm-BIP-5 | SEQ ID NO: 152 | GCATATTTTGGAACTGGCGGACGATATTGTCCTCCAC |
| | | | | ACGC |
| NG | rsmB | rsm-LF-5 | SEQ ID NO: 153 | ACGCCGAAGTCCTGT |
| NG | rsmB | rsm-LB-5 | SEQ ID NO: 154 | CGTTACCGCCTTGGAC |
| NG | rsmB | rsm-F3-6 | SEQ ID NO: 155 | GCAATGCCGAAAGCTATTTGG |
| NG | rsmB | rsm-B3-6 | SEQ ID NO: 156 | AAATCCGTTCGCCGTCTTTC |
| NG | rsmB | rsm-FlP-6 | SEQ ID NO: 157 | GGCTTCTTCCAACGTAACCGCATTGGTGGCGGAAGG |
| | | | | TATCG |
| NG | rsmB | rsm-BIP-6 | SEQ ID NO: 158 | TGCCCGGTTTTGCCGAAGGTAAGGAGATACGCCGCC |
| | | | | TG |
| NG | rsmB | rsm-LF-6 | SEQ ID NO: 159 | ATTCGTCCAACGCCTTAGCC |
| NG | rsmB | rsm-LB-6 | SEQ ID NO: 160 | ACTGGTGTCGGTACAGGACTT |
| NG | rsmB | rsm-BIP-s3bis | SEQ ID NO: 161 | CCGTCTGCCCGGTCGCCGAAGTCCTGT |
| NG | rplF | rplF-F3-s1 | SEQ ID NO: 162 | TTGGAACAGAGGCATTGGT |
| NG | rplF | rplF-B3-s1 | SEQ ID NO: 163 | CAACTTGTTTATCCGAGCCAG |
| NG | rplF | rplF-FIP-s1 | SEQ ID NO: 164 | GCTGACTAATGCGCGAGCAGGCATTCTGATGTAGCC |
| | | | | ATTGAA |
| NG | rplF | rplF-BIP-s1 | SEQ ID NO: 165 | GGTTATCGTGCTCAAGCACAAGGTCTGTTTGGCTAG |
| | | | | GAGTTTGA |
| NG | rplF | rplF-LF-s1 | SEQ ID NO: 166 | ACCAGACATTGCATTTGCTTGT |
| NG | rplF | rplF-LB-s1 | SEQ ID NO: 167 | TGCCTGAAGGTGTCTCCG |
| NG | rplF | rplF-F3-s2 | SEQ ID NO: 168 | GCATTCTGATGTAGCCATTG |
| NG | rplF | rplF-B3-s2 | SEQ ID NO: 169 | CTCTGTTTGGCTAGGAGT |
| NG | rplF | rplF-FIP-52 | SEQ ID NO: 170 | TCTTCTCAAAACCTTCTGAAACACCAAGCAAATGCAA |
| | | | | TGTCTGG |
| NG | rplF | rplF-BIP-52 | SEQ ID NO: 171 | CGTGCTCAAGCACAAGGTAATTGAACGGAGACACCT |
| | | | | TC |
| NG | rplF | rplF-LF-s2 | SEQ ID NO: 172 | ACCATATTGCTGACTAATGCG |
| NG | rplF | rplF-LB-s2 | SEQ ID NO: 173 | ATCCGATCGTATATGAAATGCC |
| NG | rplF | rplF-F3-s3 | SEQ ID NO: 174 | GCAGTAAACAAGCAAATGC |
| NG | rplF | rplF-FIP-s3 | SEQ ID NO: 175 | TCTTCTCAAAACCTTCTGAAACACCAATGTCTGGTAC |
| | | | | TGCTCG |
| NG | rplF | rplF-FIP-s4 | SEQ ID NO: 176 | CTTCTCAAAACCTTCTGAAACACCTAATGTCTGGTAC |
| | | | | TGCTCG |
| NG | rplF | rplF-FIP-s5 | SEQ ID NO: 177 | TCTTCTCAAAACCTTCTGAAACACCGCAGTAAACAAG |
| | | | | CAAATGC |
| NG | rplF | rplF-F3-s6 | SEQ ID NO: 178 | TTGGAACAGAGGCATTGGT |
| NG | rplF | rplF-BIP-s6s4 | SEQ ID NO: 179 | CAACTTGTTTATCCGAGCCAG |

### Probes

Detection of the LAMP amplified products can be achieved via a variety of methods. In a preferred embodiment, detection of product is conducted by adding a fluorescently-labeled probe to the primer mix. The term used herein “probe” refers to a single-stranded nucleic acid molecule comprising a portion or portions that are complementary, or substantially complementary, to a target sequence. In certain implementations, the fluorescently-labeled probe is a molecular beacon.

The molecular beacon can be composed of nucleic acid only such as DNA or RNA, or it can be composed of a peptide nucleic acid (PNA) conjugate. The fluorophore can be any fluorescent organic dye or a single quantum dot. The quenching moiety desirably quenches the luminescence of the fluorophore. Any suitable quenching moiety that quenches the luminescence of the fluorophore can be used. A fluorophore can be any fluorescent marker/dye known in the art. Examples of suitable fluorescent markers include, but are not limited to, Fam, Hex, Tet, Joe, Rox, Tamra, Max, Edans, Cy dyes such as Cy5, Fluorescein, Coumarin, Eosine, Rhodamine, Bodipy, Alexa, Cascade Blue, Yakima Yellow, Lucifer Yellow, Texas Red, and the family of ATTO dyes. A quencher can be any quencher known in the art. Examples of quenchers include, but are not limited to, Dabcyl, Dark Quencher, Eclipse Dark Quencher, ElleQuencher, Tamra, BHQ and QSY (all of them are Trade-Marks). The skilled person would know which combinations of dye/quencher are suitable when designing a probe. In an exemplary embodiment, fluorescein (FAM) is used in conjunction with Blackhole Quencher™ (BHQ™) (Novato, Calif). Binding of the molecular beacon to amplified product can then be directly, visually assessed. Alternatively, the fluorescence level can be measured by spectroscopy in order to improve sensitivity.

A variety of commercial suppliers produce standard and custom molecular beacons, including Abingdon Health (UK; www.abingdonhealth.com), Attostar (US, MN; www.attostar.com), Biolegio (NLD; www.biolegio.cont), Biomers.net (DEU; www.biomers.net), Biosearch Technologies (US, CA; www.biosearchtech.com), Eurogentec (BEL; www.eurogentec.com), Gene Link (US, NY; www.genelink.com) Integrated DNA Technologies (US, IA; www.idtdna.com), Isogen Life Science (NLD; www.isogen-lifescience.com), Midland Certified Reagent (US, TX; www.oligos.com), Eurofins (DEU; www.eurofinsgenomics.eu), Sigma-Aldrich (US, TX; www.sigmaaldrich.com), Thermo Scientific (US, MA; www.thermoscientific.com), TIB MOLBIOL (DEU; www.tib-molbiol.de), TriLink Bio Technologies (US, CA; www.trilinkbiotech.com). A variety of kits, which utilize molecular beacons are also commercially available, such as the Sentinel™ Molecular Beacon Allelic Discrimination Kits from Stratagene (La Jolla, Calif.) and various kits from Eurogentec SA (Belgium, eurogentec.com) and Isogen Bioscience BV (The Netherlands, isogen.com).

The oligonucleotide probes and primers of the invention are optionally prepared using essentially any technique known in the art. In certain embodiments, for example, the oligonucleotide probes and primers described herein are synthesized chemically using essentially any nucleic acid synthesis method, including, e.g., according to the solid phase phosphoramidite triester method described by Beaucage and Caruthers (1981), Tetrahedron Setts. 22(20):1859-1862, which is incorporated by reference, or another synthesis technique known in the art, e.g., using an automated synthesizer, as described in Needham-VanDevanter et al. (1984) Nucleic Acids Res. 12:6159-6168, which is incorporated by reference. A wide variety of equipment is commercially available for automated oligonucleotide synthesis. Multi-nucleotide synthesis approaches (e.g., tri-nucleotide synthesis, etc.) are also optionally utilized. Moreover, the primer nucleic acids described herein optionally include various modifications. To further illustrate, primers are also optionally modified to improve the specificity of amplification reactions as described in, e.g., U.S. Pat. No. 6,001,611, issued Dec. 14, 1999, which is incorporated by reference. Primers and probes can also be synthesized with various other modifications as described herein or as otherwise known in the art.

In addition, essentially any nucleic acid (and virtually any labeled nucleic acid, whether standard or non-standard) can be custom or standard ordered from any of a variety of commercial sources, such as Integrated DNA Technologies, the Midland Certified Reagent Company, Eurofins, Biosearch Technologies, Sigma Aldrich and many others.

### Test Samples

Test samples are generally derived or isolated from subjects, typically mammalian subjects, more typically human subjects. In some embodiments, the subjects are suspected of hosting an infectious agent, for example, having a Chlamydia infection or N. Gonorrhoeae infection. Exemplary samples or specimens include blood, plasma, serum, urine, feces, synovial fluid, spinal fluid, seminal fluid, seminal plasma, prostatic fluid, vaginal fluid, cervical fluid, uterine fluid, cervical scrapings, amniotic fluid, anal scrapings, mucus, sputum, tissue, and the like. Essentially any technique for acquiring these samples is optionally utilized including, e.g., scraping, venipuncture, swabbing, biopsy, or other techniques known in the art.

The term “infectious agent” refers to any organism or microorganism, including bacteria, yeast, fungi, viruses, protists (protozoan, micro-algae), archaebacteria, and eukaryotes that infiltrates another living thing (the host). The term “infectious agent” refers to living matter and viruses comprising nucleic acid that can be detected and identified by the methods of the invention. Examples of infectious agents include bacterial pathogens such as: Aeromonas hydrophila and other species (spp.); Bacillus anthraces; Bacillus cereus; Botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia trachomatis; Clostridium botulinum; Clostridium botulinum; Clostridium perfringens; Coccidioides immitis; Coccidioides posadasii; Cowdria ruminantium (Heartwater); Coxiella burnetii; Enterovirulent Escherichia co//group (EEC Group) such as Escherichia coli—enterotoxigenic (ETEC), Escherichia coli—enteropathogenic (EPEC), Escherichia coli—O157:H7 enterohemorrhagic (EHEC), and Escherichia coli—enteroinvasive (EIEC); Ehrlichia spp. such as Ehrlichia chaffeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Neisseria gonorrhoeae, Peronosclerospora philippinensis; Phakopsora pachyrhizi; Plesiomonas shigelloides; Ralstonia solanacearum race 3, biovar 2; Rickettsia prowazekii; Rickettsia rickettsia; Salmonella spp.; Schlerophthora rayssiae varzeae; Shigella spp.; Staphylococcus aureus; Streptococcus; Synchytrium endobioticum; Vibrio cholerae non-O1; Vibrio cholerae O1; Vibrio parahaemolyticus and other Vibrios; Vibrio vulnificus; Xanthomonas oryzae; Xylella fastidiosa (citrus variegated chlorosis strain); Yersinia enterocolitica and Yersinia pseudotuberculosis; and Yersinia pestis. Further examples of organisms include viruses such as: African horse sickness virus; African swine fever virus; Akabane virus; Avian influenza virus (highly pathogenic); Bhanja virus; Blue tongue virus (Exotic); Camel pox virus; Cercopithecine herpesvirus 1 ; Chikungunya virus; Classical swine fever virus; Coronavirus (SARS); Crimean-Congo hemorrhagic fever virus; Dengue viruses; Dugbe virus; Ebola viruses; Encephalitic viruses such as Eastern equine encephalitis virus, Japanese encephalitis virus, Murray Valley encephalitis, and Venezuelan equine encephalitis virus; Equine morbillivirus; Flexal virus; Foot and mouth disease virus; Germiston virus; Goat pox virus; Hantaan or other Hanta viruses; Hendra virus; Issyk-kul virus; Koutango virus; Lassa fever virus; Louping ill virus; Lumpy skin disease virus; Lymphocytic choriomeningitis virus; Malignant catarrhal fever virus (Exotic); Marburg virus; Mayaro virus; Menangle virus; Monkeypox virus; Mucambo virus; Newcastle disease virus (WND); Nipah Virus; Norwalk virus group; Oropouche virus; Orungo virus; Peste Des Petits Ruminants virus; Piry virus; Plum Pox Potyvirus; Poliovirus; Potato virus; Powassan virus; Rift Valley fever virus; Rinderpest virus; Rotavirus; Semliki Forest virus; Sheep pox virus; South American hemorrhagic fever viruses such as Flexal, Guanarito, Junin, Machupo, and Sabia; Spondweni virus; Swine vesicular disease virus; Tickborne encephalitis complex (flavi) viruses such as Central European tickborne encephalitis, Far Eastern tick-borne encephalitis, Russian spring and summer encephalitis, Kyasanur forest disease, and Omsk hemorrhagic fever; Variola major virus (Smallpox virus); Variola minor virus (Alastrim); Vesicular stomatitis virus (Exotic); Wesselbron virus; West Nile virus; Yellow fever virus; and South American hemorrhagic fever viruses such as Junin, Machupo, Sabia, Flexal, and Guanarito.

Advantageously, the invention enables reliable rapid detection of target nucleic acids in a test sample. In some embodiments, the test sample is clinical sample, such as a urine sample.

To further illustrate, prior to analyzing the target nucleic acids described herein, those nucleic acids may be purified or isolated from samples that typically include complex mixtures of different components. Cells in collected samples are typically lysed to release the cell contents. For example, cells in the biological sample can be lysed by contacting them with various enzymes, chemicals, and/or lysed by other approaches known in the art, which degrade, e.g., bacterial cell walls. In some embodiments, nucleic acids are analyzed directly in the cell lysate. In other embodiments, nucleic acids are further purified or extracted from cell lysates prior to detection. Essentially any nucleic acid extraction methods can be used to purify nucleic acids in the samples utilized in the methods of the present invention. Exemplary techniques that can be used to purifying nucleic acids include, e.g., affinity chromatography, hybridization to probes immobilized on solid supports, liquid-liquid extraction (e.g., phenol-chloroform extraction, etc.), precipitation (e.g., using ethanol, etc.), extraction with filter paper, extraction with micelle-forming reagents (e.g., cetyl-trimethyl-ammonium-bromide, etc.), binding to immobilized intercalating dyes (e.g., ethidium bromide, acridine, etc.), adsorption to silica gel or diatomic earths, adsorption to magnetic glass particles or organo silane particles under chaotropic conditions, and/or the like. Sample processing is also described in, e.g., U.S. Pat. Nos. 5,155,018, 6,383,393, and 5,234,809, which are each incorporated by reference.

A test sample may optionally have been treated and/or purified according to any technique known by the skilled person, to improve the amplification efficiency and/or qualitative accuracy and/or quantitative accuracy. The sample may thus exclusively, or essentially, consist of nucleic acid(s), whether obtained by purification, isolation, or by chemical synthesis. Means are available to the skilled person, who would like to isolate or purify nucleic acids, such as DNA, from a test sample, for example to isolate or purify DNA from cervical scrapes (e.g., QIAamp-DNA Mini-Kit; Qiagen, Hilden, Germany).

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B(1992).

### Example 1: Target Selection and Primer Probe Design

16S and 23S gene sequences for multiple serovars of C. trachomatis, closely related species such as Chlamydophila pneumoniae and Chlamydia psittasci, and for other species commonly found in the urine or vaginal fluid were retrieved from the NCBI database. Sequences were aligned using Clustal omega (Sievers, et al. 2011. Molecular Systems Biology 7:539) and regions with unique specific bases to C. trachomatis species were identified. Loop mediated amplification primers were designed using LAMP designer (Premier Biosoft). For added specificity, molecular beacons or probes targeting the amplified products were designed manually or using Beacon designer (Premier Biosoft). Designed primer sets and beacons were further analyzed for specificity using BLAST against the human genome and the NCBI nucleotide database. Various primer sets and probes were designed and screened for reaction speed.

Sequences for Neisseria gonorrhoeae and closely related species including Neisseria meningitidis, Neisseria lactamica, and Neisseria sicca were obtained from the National Center for Biotechnology Information (NCBI) or Pathosystems Resource Integration Center (PATRIC) databases. Sequences were aligned using Clustal Omega (Sievers, et al. (2011). Molecular Systems Biology 7:539) or MAFFT (Katoh, Standley 2013. Molecular Biology and Evolution 30:772-780) and regions unique to N. gonorrhoeae were selected for primer and molecular beacon probe design.

Primer/probe based detection assays were designed to utilize isothermal loop mediated amplification (LAMP) targeting RNA through the addition of a Reverse transcriptase (RT-LAMP) to the reaction. A molecular beacon probe with 5′ fluorophore/3′ quencher modifications (6-Carboxyfluorescein and Black Hole Quencher 1 in most instances or Atto 565N and Black Hole Quencher 2 where indicated) was included to provide target-specific fluorescent detection. N gonorrhoeae and C. trachomatis RT-LAMP primer sets (Table 1 and Table 2) were designed using a combination of software programs including PremierBiosoft's LAMP Designer, Beacon Designer, an in-house command line based script and manual designs. Resulting assay amplicons and molecular beacons were additionally Blasted against the NCBI nucleotide database, including the human transcriptome, and against individual non-gonorrhoeae species within the genus Neisseria to further predict assay specificity.

The inventive primer sets for both C. trachomatis (CT) and N. gonorrhoeae (NG) and closely related species are summarized in Table 2, which include, at a minimum, a forward inner primer (FIP) and backward inner primer (BIP). Additionally, the primer sets typically also include at least two additional primers selected from the forward outer primer (F3), backward outer primer (B3), forward loop primer (LF) and backward loop primer (LB).

TABLE 2
LAMP Primer Sets

| Organ- | Tar- | Primer | Primer | | | | | | |
|---|---|---|---|---|---|---|---|---|---|
| ism | get | Set ID | Set # | F3 | B3 | FIP | BIP | LF | LB |
| CT | 23S | Ps6 | Set-1 | SEQ ID NO: 1 | SEQ ID NO: 2 | SEQ ID NO: 3 | SEQ ID NO: 4 | SEQ ID NO: 5 | SEQ ID NO: 6 |
| CT | 23S | Ps7 | Set-2 | SEQ ID NO: 1 | SEQ ID NO: 2 | SEQ ID NO: 7 | SEQ ID NO: 4 | SEQ ID NO: 8 | SEQ ID NO: 6 |
| CT | 23S | Ps8 | Set-3 | SEQ ID NO: 9 | SEQ ID NO: 10 | SEQ ID NO: 11 | SEQ ID NO: 12 | SEQ ID NO: 8 | SEQ ID NO: 13 |
| CT | 23S | Ps11 | Set-4 | SEQ ID NO: 14 | SEQ ID NO: 15 | SEQ ID NO: 16 | SEQ ID NO: 17 | SEQ ID NO: 5 | SEQ ID NO: 18 |
| CT | 23S | Ps12 | Set-5 | SEQ ID NO: 19 | SEQ ID NO: 20 | SEQ ID NO: 21 | SEQ ID NO: 22 | SEQ ID NO: 23 | SEQ ID NO: 24 |
| CT | 23S | Ps13 | Set-6 | SEQ ID NO: 25 | SEQ ID NO: 26 | SEQ ID NO: 27 | SEQ ID NO: 22 | SEQ ID NO: 29 | SEQ ID NO: 30 |
| CT | 23S | Pn2 | Set-7 | SEQ ID NO: 31 | SEQ ID NO: 32 | SEQ ID NO: 33 | SEQ ID NO: 34 | SEQ ID NO: 35 | SEQ ID NO: 36 |
| CT | 23S | Pn3 | Set-8 | SEQ ID NO: 37 | SEQ ID NO: 38 | SEQ ID NO: 39 | SEQ ID NO: 40 | SEQ ID NO: 41 | SEQ ID NO: 42 |
| CT | 23S | Ps1 | Set-9 | SEQ ID NO: 43 | SEQ ID NO: 44 | SEQ ID NO: 45 | SEQ ID NO: 46 | SEQ ID NO: 5 | SEQ ID NO: 47 |
| CT | 23S | Ps2(1) | Set-10 | SEQ ID NO: 48 | SEQ ID NO: 49 | SEQ ID NO: 50 | SEQ ID NO: 51 | SEQ ID NO: 52 | SEQ ID NO: 53 |
| CT | 23S | Ps7 | Set-11 | SEQ ID NO: 54 | SEQ ID NO: 55 | SEQ ID NO: 56 | SEQ ID NO: 57 | SEQ ID NO: 8 | SEQ ID NO: 58 |
| CT | 23S | Ps12 | Set-12 | SEQ ID NO: 59 | SEQ ID NO: 60 | SEQ ID NO: 61 | SEQ ID NO: 62 | SEQ ID NO: 63 | SEQ ID NO: 64 |
| CT | 23S | Ps14 | Set-13 | SEQ ID NO: 65 | SEQ ID NO: 66 | SEQ ID NO: 67 | SEQ ID NO: 68 | SEQ ID NO: 69 | SEQ ID NO: 70 |
| CT | 23S | Pn1 | Set-14 | SEQ ID NO: 71 | SEQ ID NO: 72 | SEQ ID NO: 73 | SEQ ID NO: 74 | SEQ ID NO: 75 | SEQ ID NO: 76 |
| CT | 23S | Ps6 | Set-15 | SEQ ID NO: 1 | SEQ ID NO: 2 | SEQ ID NO: 3 | SEQ ID NO: 4 | — | — |
| CT | 23S | Ps7 | Set-16 | SEQ ID NO: 1 | SEQ ID NO: 2 | SEQ ID NO: 7 | SEQ ID NO: 4 | — | — |
| CT | 23S | Ps8 | Set-17 | SEQ ID NO: 9 | SEQ ID NO: 10 | SEQ ID NO: 11 | SEQ ID NO: 12 | — | — |
| CT | 23S | Ps11 | Set-18 | SEQ ID NO: 14 | SEQ ID NO: 15 | SEQ ID NO: 16 | SEQ ID NO: 17 | — | — |
| CT | 23S | Ps12 | Set-19 | SEQ ID NO: 19 | SEQ ID NO: 20 | SEQ ID NO: 21 | SEQ ID NO: 22 | — | — |
| CT | 23S | Ps13 | Set-20 | SEQ ID NO: 25 | SEQ ID NO: 26 | SEQ ID NO: 27 | SEQ ID NO: 28 | — | — |
| CT | 23S | Pn2 | Set-21 | SEQ ID NO: 31 | SEQ ID NO: 32 | SEQ ID NO: 33 | SEQ ID NO: 34 | — | — |
| CT | 23S | Pn3 | Set-22 | SEQ ID NO: 37 | SEQ ID NO: 38 | SEQ ID NO: 39 | SEQ ID NO: 40 | — | — |
| CT | 23S | Ps1 | Set-23 | SEQ ID NO: 43 | SEQ ID NO: 44 | SEQ ID NO: 45 | SEQ ID NO: 46 | — | — |
| CT | 23S | Ps2(1) | Set-24 | SEQ ID NO: 48 | SEQ ID NO: 49 | SEQ ID NO: 50 | SEQ ID NO: 51 | — | — |
| CT | 23S | Ps7 | Set-25 | SEQ ID NO: 54 | SEQ ID NO: 55 | SEQ ID NO: 56 | SEQ ID NO: 57 | — | — |
| CT | 23S | Ps14 | Set-26 | SEQ ID NO: 65 | SEQ ID NO: 66 | SEQ ID NO: 67 | SEQ ID NO: 68 | — | — |
| CT | 23S | Pn1 | Set-27 | SEQ ID NO: 71 | SEQ ID NO: 72 | SEQ ID NO: 73 | SEQ ID NO: 74 | — | — |
| CT | 23S | Ps6 | Set-28 | — | — | SEQ ID NO: 3 | SEQ ID NO: 4 | SEQ ID NO: 5 | SEQ ID NO: 6 |
| CT | 23S | Ps7 | Set-29 | — | — | SEQ ID NO: 7 | SEQ ID NO: 4 | SEQ ID NO: 8 | SEQ ID NO: 6 |
| CT | 23S | Ps8 | Set-30 | — | — | SEQ ID NO: 11 | SEQ ID NO: 12 | SEQ ID NO: 8 | SEQ ID NO: 13 |
| CT | 23S | Ps11 | Set-31 | — | — | SEQ ID NO: 16 | SEQ ID NO: 17 | SEQ ID NO: 5 | SEQ ID NO: 18 |
| CT | 23S | Ps12 | Set-32 | — | — | SEQ ID NO: 21 | SEQ ID NO: 22 | SEQ ID NO: 23 | SEQ ID NO: 24 |
| CT | 23S | Ps13 | Set-33 | — | — | SEQ ID NO: 27 | SEQ ID NO: 28 | SEQ ID NO: 29 | SEQ ID NO: 30 |
| CT | 23S | Pn2 | Set-34 | — | — | SEQ ID NO: 33 | SEQ ID NO: 34 | SEQ ID NO: 35 | SEQ ID NO: 36 |
| CT | 23S | Pn3 | Set-35 | — | — | SEQ ID NO: 39 | SEQ ID NO: 40 | SEQ ID NO: 41 | SEQ ID NO: 42 |
| CT | 23S | Ps1 | Set-36 | — | — | SEQ ID NO: 45 | SEQ ID NO: 46 | SEQ ID NO: 5 | SEQ ID NO: 47 |
| CT | 23S | Ps2(1) | Set-37 | — | — | SEQ ID NO: 50 | SEQ ID NO: 51 | SEQ ID NO: 52 | SEQ ID NO: 53 |
| CT | 23S | Ps7 | Set-38 | — | — | SEQ ID NO: 56 | SEQ ID NO: 57 | SEQ ID NO: 8 | SEQ ID NO: 58 |
| CT | 23S | Ps14 | Set-39 | — | — | SEQ ID NO: 67 | SEQ ID NO: 68 | SEQ ID NO: 69 | SEQ ID NO: 70 |
| CT | 23S | Pn1 | Set-40 | — | — | SEQ ID NO: 73 | SEQ ID NO: 74 | SEQ ID NO: 75 | SEQ ID NO: 76 |
| CT | 23S | Ps6 | Set-41 | — | — | SEQ ID NO: 3 | SEQ ID NO: 4 | — | — |
| CT | 23S | Ps7 | Set-42 | — | — | SEQ ID NO: 7 | SEQ ID NO: 4 | — | — |
| CT | 23S | Ps8 | Set-43 | — | — | SEQ ID NO: 11 | SEQ ID NO: 12 | — | — |
| CT | 23S | Ps11 | Set-44 | — | — | SEQ ID NO: 16 | SEQ ID NO: 17 | — | — |
| CT | 23S | Ps12 | Set-45 | — | — | SEQ ID NO: 21 | SEQ ID NO: 22 | — | — |
| CT | 23S | Ps13 | Set-46 | — | — | SEQ ID NO: 27 | SEQ ID NO: 28 | — | — |
| CT | 16S | Ps1 | Set-47 | SEQ ID NO: 77 | SEQ ID NO: 78 | SEQ ID NO: 79 | SEQ ID NO: 80 | SEQ ID NO: 81 | SEQ ID NO: 82 |
| CT | 16S | Ps6 | Set-48 | SEQ ID NO: 83 | SEQ ID NO: 84 | SEQ ID NO: 85 | SEQ ID NO: 86 | SEQ ID NO: 87 | SEQ ID NO: 88 |
| CT | 16S | Ps7 | Set-49 | SEQ ID NO: 89 | SEQ ID NO: 90 | SEQ ID NO: 91 | SEQ ID NO: 92 | SEQ ID NO: 93 | SEQ ID NO: 94 |
| CT | 16S | Ps1 | Set-50 | SEQ ID NO: 77 | SEQ ID NO: 78 | SEQ ID NO: 79 | SEQ ID NO: 80 | — | — |
| CT | 16S | Ps6 | Set-51 | SEQ ID NO: 83 | SEQ ID NO: 84 | SEQ ID NO: 85 | SEQ ID NO: 86 | — | — |
| CT | 16S | Ps7 | Set-52 | SEQ ID NO: 89 | SEQ ID NO: 90 | SEQ ID NO: 91 | SEQ ID NO: 92 | — | — |
| CT | 16S | Ps1 | Set-53 | — | — | SEQ ID NO: 79 | SEQ ID NO: 80 | SEQ ID NO: 81 | SEQ ID NO: 82 |
| CT | 16S | Ps6 | Set-54 | — | — | SEQ ID NO: 85 | SEQ ID NO: 86 | SEQ ID NO: 87 | SEQ ID NO: 88 |
| CT | 16S | Ps7 | Set-55 | — | — | SEQ ID NO: 91 | SEQ ID NO: 92 | SEQ ID NO: 93 | SEQ ID NO: 94 |
| CT | 16S | Ps1 | Set-56 | — | — | SEQ ID NO: 79 | SEQ ID NO: 80 | — | — |
| CT | 16S | Ps6 | Set-57 | — | — | SEQ ID NO: 85 | SEQ ID NO: 86 | — | — |
| CT | 16S | Ps7 | Set-58 | — | — | SEQ ID NO: 91 | SEQ ID NO: 92 | — | — |
| NG | 23S | Pn2 | Set-59 | SEQ ID NO: 95 | SEQ ID NO: 96 | SEQ ID NO: 97 | SEQ ID NO: 98 | SEQ ID NO: 99 | SEQ ID NO: 100 |
| NG | 23S | Pn2 | Set-90 | SEQ ID NO: 95 | SEQ ID NO: 96 | SEQ ID NO: 97 | SEQ ID NO: 98 | SEQ ID NO: 99 | SEQ ID NO: 100 |
| NG | 23S | Ps1 | Set-60 | SEQ ID NO: 101 | SEQ ID NO: 102 | SEQ ID NO: 103 | SEQ ID NO: 104 | SEQ ID NO: 105 | SEQ ID NO: 106 |
| NG | 23S | Ps2 | Set-61 | SEQ ID NO: 107 | SEQ ID NO: 108 | SEQ ID NO: 109 | SEQ ID NO: 110 | SEQ ID NO: 111 | SEQ ID NO: 112 |
| NG | 23S | Pnew2 | Set-62 | SEQ ID NO: 107 | SEQ ID NO: 108 | SEQ ID NO: 109 | SEQ ID NO: 113 | SEQ ID NO: 111 | SEQ ID NO: 114 |
| NG | 23S | Ps3 | Set-63 | SEQ ID NO: 115 | SEQ ID NO: 116 | SEQ ID NO: 117 | SEQ ID NO: 118 | SEQ ID NO: 119 | SEQ ID NO: 120 |
| NG | 23S | Ps1 | Set-64 | SEQ ID NO: 101 | SEQ ID NO: 102 | SEQ ID NO: 103 | SEQ ID NO: 104 | SEQ ID NO: 105 | SEQ ID NO: 106 |
| NG | 23S | Pn2 | Set-65 | SEQ ID NO: 95 | SEQ ID NO: 96 | SEQ ID NO: 97 | SEQ ID NO: 98 | — | — |
| NG | 23S | Ps1 | Set-66 | SEQ ID NO: 101 | SEQ ID NO: 102 | SEQ ID NO: 103 | SEQ ID NO: 104 | — | — |
| NG | 23S | Ps2 | Set-67 | SEQ ID NO: 107 | SEQ ID NO: 108 | SEQ ID NO: 109 | SEQ ID NO: 110 | — | — |
| NG | 23S | Pnew2 | Set-68 | SEQ ID NO: 107 | SEQ ID NO: 108 | SEQ ID NO: 109 | SEQ ID NO: 113 | — | — |
| NG | 23S | Ps3 | Set-69 | SEQ ID NO: 115 | SEQ ID NO: 116 | SEQ ID NO: 117 | SEQ ID NO: 118 | — | — |
| NG | 23S | Ps1 | Set-70 | SEQ ID NO: 101 | SEQ ID NO: 102 | SEQ ID NO: 103 | SEQ ID NO: 104 | — | — |
| NG | 23S | Pn2 | Set-71 | — | — | SEQ ID NO: 97 | SEQ ID NO: 98 | SEQ ID NO: 99 | SEQ ID NO: 100 |
| NG | 23S | Ps1 | Set-72 | — | — | SEQ ID NO: 103 | SEQ ID NO: 104 | SEQ ID NO: 105 | SEQ ID NO: 106 |
| NG | 23S | Ps2 | Set-73 | — | — | SEQ ID NO: 109 | SEQ ID NO: 110 | SEQ ID NO: 111 | SEQ ID NO: 112 |
| NG | 23S | Pnew2 | Set-74 | — | — | SEQ ID NO: 109 | SEQ ID NO: 113 | SEQ ID NO: 111 | SEQ ID NO: 114 |
| NG | 23S | Ps3 | Set-75 | — | — | SEQ ID NO: 117 | SEQ ID NO: 118 | SEQ ID NO: 119 | SEQ ID NO: 120 |
| NG | 23S | Ps1 | Set-76 | — | — | SEQ ID NO: 103 | SEQ ID NO: 104 | SEQ ID NO: 105 | SEQ ID NO: 106 |
| NG | 16S | Pn1 | Set-77 | SEQ ID NO: 121 | SEQ ID NO: 122 | SEQ ID NO: 123 | SEQ ID NO: 124 | SEQ ID NO: 125 | SEQ ID NO: 126 |
| NG | 16S | Pn1 | Set-78 | SEQ ID NO: 121 | SEQ ID NO: 122 | SEQ ID NO: 123 | SEQ ID NO: 124 | — | — |
| NG | 16S | Pn1 | Set-79 | — | — | SEQ ID NO: 123 | SEQ ID NO: 124 | SEQ ID NO: 125 | SEQ ID NO: 126 |
| NG | rsmB | Ps1 | Set-80 | SEQ ID NO: 127 | SEQ ID NO: 128 | SEQ ID NO: 129 | SEQ ID NO: 130 | SEQ ID NO: 131 | SEQ ID NO: 132 |
| NG | rsmB | Ps2 | Set-81 | SEQ ID NO: 133 | SEQ ID NO: 134 | SEQ ID NO: 135 | SEQ ID NO: 136 | SEQ ID NO: 137 | SEQ ID NO: 138 |
| NG | rsmB | Ps3 | Set-82 | SEQ ID NO: 139 | SEQ ID NO: 128 | SEQ ID NO: 140 | SEQ ID NO: 141 | SEQ ID NO: 142 | SEQ ID NO: 143 |
| NG | rsmB | Ps4 | Set-83 | SEQ ID NO: 144 | SEQ ID NO: 145 | SEQ ID NO: 146 | SEQ ID NO: 147 | SEQ ID NO: 148 | SEQ ID NO: 149 |
| NG | rsmB | Ps5 | Set-84 | SEQ ID NO: 150 | SEQ ID NO: 145 | SEQ ID NO: 151 | SEQ ID NO: 152 | SEQ ID NO: 153 | SEQ ID NO: 154 |
| NG | rsmB | Ps6 | Set-85 | SEQ ID NO: 155 | SEQ ID NO: 156 | SEQ ID NO: 157 | SEQ ID NO: 158 | SEQ ID NO: 159 | SEQ ID NO: 160 |
| NG | rsmB | Ps3 | Set-86 | SEQ ID NO: 139 | SEQ ID NO: 128 | SEQ ID NO: 140 | SEQ ID NO: 141 | SEQ ID NO: 142 | SEQ ID NO: 143 |
| NG | rsmB | Ps4 | Set-87 | SEQ ID NO: 144 | SEQ ID NO: 145 | SEQ ID NO: 146 | SEQ ID NO: 147 | SEQ ID NO: 148 | SEQ ID NO: 149 |
| NG | rsmB | Ps5 | Set-88 | SEQ ID NO: 150 | SEQ ID NO: 145 | SEQ ID NO: 151 | SEQ ID NO: 152 | SEQ ID NO: 153 | SEQ ID NO: 154 |
| NG | rsmB | Ps6 | Set-89 | SEQ ID NO: 155 | SEQ ID NO: 156 | SEQ ID NO: 157 | SEQ ID NO: 158 | SEQ ID NO: 159 | SEQ ID NO: 160 |
| NG | rsmB | Ps3bis | Set-91 | SEQ ID NO: 139 | SEQ ID NO: 128 | SEQ ID NO: 140 | SEQ ID NO: 161 | SEQ ID NO: 142 | SEQ ID NO: 143 |
| NG | rsmB | Ps1 | Set-92 | SEQ ID NO: 127 | SEQ ID NO: 128 | SEQ ID NO: 129 | SEQ ID NO: 130 | — | — |
| NG | rsmB | Ps2 | Set-93 | SEQ ID NO: 133 | SEQ ID NO: 134 | SEQ ID NO: 135 | SEQ ID NO: 136 | — | — |
| NG | rsmB | Ps3 | Set-94 | SEQ ID NO: 139 | SEQ ID NO: 128 | SEQ ID NO: 140 | SEQ ID NO: 141 | — | — |
| NG | rsmB | Ps4 | Set-95 | SEQ ID NO: 144 | SEQ ID NO: 145 | SEQ ID NO: 146 | SEQ ID NO: 147 | — | — |
| NG | rsmB | Ps5 | Set-96 | SEQ ID NO: 150 | SEQ ID NO: 145 | SEQ ID NO: 151 | SEQ ID NO: 152 | — | — |
| NG | rsmB | Ps6 | Set-97 | SEQ ID NO: 155 | SEQ ID NO: 156 | SEQ ID NO: 157 | SEQ ID NO: 158 | — | — |
| NG | rsmB | Ps3 | Set-98 | SEQ ID NO: 139 | SEQ ID NO: 128 | SEQ ID NO: 140 | SEQ ID NO: 141 | — | — |
| NG | rsmB | Ps4 | Set-99 | SEQ ID NO: 144 | SEQ ID NO: 145 | SEQ ID NO: 146 | SEQ ID NO: 147 | — | — |
| NG | rsmB | Ps5 | Set-100 | SEQ ID NO: 150 | SEQ ID NO: 145 | SEQ ID NO: 151 | SEQ ID NO: 152 | — | — |
| NG | rsmB | Ps6 | Set-101 | SEQ ID NO: 155 | SEQ ID NO: 156 | SEQ ID NO: 157 | SEQ ID NO: 158 | — | — |
| NG | rsmB | Pn2 | Set-102 | SEQ ID NO: 95 | SEQ ID NO: 96 | SEQ ID NO: 97 | SEQ ID NO: 98 | — | — |
| NG | rsmB | Ps1 | Set-103 | — | — | SEQ ID NO: 129 | SEQ ID NO: 130 | SEQ ID NO: 131 | SEQ ID NO: 132 |
| NG | rsmB | Ps2 | Set-104 | — | — | SEQ ID NO: 135 | SEQ ID NO: 136 | SEQ ID NO: 137 | SEQ ID NO: 138 |
| NG | rsmB | Ps3 | Set-105 | — | — | SEQ ID NO: 140 | SEQ ID NO: 141 | SEQ ID NO: 142 | SEQ ID NO: 143 |
| NG | rsmB | Ps4 | Set-106 | — | — | SEQ ID NO: 146 | SEQ ID NO: 147 | SEQ ID NO: 148 | SEQ ID NO: 149 |
| NG | rsmB | Ps5 | Set-107 | — | — | SEQ ID NO: 151 | SEQ ID NO: 152 | SEQ ID NO: 153 | SEQ ID NO: 154 |
| NG | rsmB | Ps6 | Set-108 | — | — | SEQ ID NO: 157 | SEQ ID NO: 158 | SEQ ID NO: 159 | SEQ ID NO: 160 |
| NG | rsmB | Ps3 | Set-109 | — | — | SEQ ID NO: 140 | SEQ ID NO: 141 | SEQ ID NO: 142 | SEQ ID NO: 143 |
| NG | rsmB | Ps4 | Set-110 | — | — | SEQ ID NO: 146 | SEQ ID NO: 147 | SEQ ID NO: 148 | SEQ ID NO: 149 |
| NG | rsmB | Ps5 | Set-111 | — | — | SEQ ID NO: 151 | SEQ ID NO: 152 | SEQ ID NO: 153 | SEQ ID NO: 154 |
| NG | rsmB | Ps6 | Set-112 | — | — | SEQ ID NO: 157 | SEQ ID NO: 158 | SEQ ID NO: 159 | SEQ ID NO: 160 |
| NG | rsmB | Pn2 | Set-113 | — | — | SEQ ID NO: 97 | SEQ ID NO: 98 | SEQ ID NO: 99 | SEQ ID NO: 100 |
| NG | rsmB | Ps1 | Set-114 | — | — | SEQ ID NO: 129 | SEQ ID NO: 130 | — | — |
| NG | rplF | Ps1 | Set-115 | SEQ ID NO: 162 | SEQ ID NO: 163 | SEQ ID NO: 164 | SEQ ID NO: 165 | SEQ ID NO: 166 | SEQ ID NO: 167 |
| NG | rplF | Ps2 | Set-116 | SEQ ID NO: 168 | SEQ ID NO: 169 | SEQ ID NO: 170 | SEQ ID NO: 171 | SEQ ID NO: 172 | SEQ ID NO: 173 |
| NG | rplF | Ps3 | Set-117 | SEQ ID NO: 174 | SEQ ID NO: 169 | SEQ ID NO: 175 | SEQ ID NO: 171 | SEQ ID NO: 172 | SEQ ID NO: 173 |
| NG | rplF | Ps4 | Set-118 | SEQ ID NO: 174 | SEQ ID NO: 169 | SEQ ID NO: 176 | SEQ ID NO: 171 | SEQ ID NO: 172 | SEQ ID NO: 173 |
| NG | rplF | Ps5 | Set-119 | SEQ ID NO: 168 | SEQ ID NO: 169 | SEQ ID NO: 177 | SEQ ID NO: 171 | SEQ ID NO: 172 | SEQ ID NO: 173 |
| NG | rplF | Ps6 | Set-120 | SEQ ID NO: 178 | SEQ ID NO: 169 | SEQ ID NO: 176 | SEQ ID NO: 179 | SEQ ID NO: 172 | SEQ ID NO: 173 |

### Example 2: Amplification Reaction Kinetics

A negative urine matrix was spiked with titred C. trachomatis (serially diluted in PBS, Zeptometrix CN#0801775) at two different concentrations (10³ IFU/mL and 10 IFU/mL). Nucleic acids were extracted using standard extraction methods and the sample was amplified using LAMP primers (SEQ ID NOs: 1-6). YoPro™ dye (Life Technologies; green fluorescent carbocyanine nucleic acid stain) was used for the detection of the amplified product. In this example, a 25 μL reaction contained 1× Isothermal Amplification Buffer (New England Biolabs) supplemented with 4.8 mM or 6 mM MgCl₂, 1.4 mM or 1.6 mM dNTP, 200nM YO-PRO-1 dye (Life Technologies), primers (2 μM of F3 and B3, when present; 1.6 μM of FIP and BIP; 8 μM of LF and LB, when present), 8 or 12 Units of Bst2 polymerase (New England Biolabs), 7.5 Units RTx Warmstart (reverse transcriptase; New England Biolabs), and the extracted nucleic acid (as template) or water (as no template control). The reactions were incubated at 63° or 65° C. and kinetics were monitored using a Roche real-time Lightcycler96 (Roche).

This example shows that using this set of primers and the loop mediated amplification method, fast amplification kinetics are achieved. Results are summarized in Table 3, in which the Time to Positive (Tp) was calculated by the instrument. Results are classified by the time to position: A having Tp in less or equal to 8 minutes, B having Tp between 8 minutes and 12 minutes (inclusive), and C having Tp greater than 12 minutes.

TABLE 3
Time to Positive Dye Detection

| | | Primer | Tₚ 10³ | Tₚ 10 |
|---|---|---|---|---|
| Organism | Target | Set # | IFU/mL | IFU/mL |
| CT | 23S | Set-1 | A | A |
| CT | 23S | Set-2 | A | B |
| CT | 23S | Set-3 | A | A |
| CT | 23S | Set-4 | B | C |
| CT | 23S | Set-5 | A | B |
| CT | 23S | Set-6 | A | A |
| CT | 16S | Set-47 | C | C |

A negative urine matrix was spiked with titred N. gonorrhoeae (serially diluted in PBS, Zeptometrix CN # 0801482) at 10 CFU/ml. Nucleic acids were extracted from the spiked sample or from negative urine using standard extraction methods and the sample was amplified using LAMP primer sets described in Table 2.

YoPro™ dye (Life Technologies; green fluorescent carbocyanine nucleic acid stain) was used for the detection of the amplified product. The master mix was prepared as described above for CT. Results are summarized in Table 4, in which the Time to Positive (Tp) was calculated by the instrument. Results are classified by the time to positive (Tp) from reaction initiation as follows: “A” indicates a Tp of less than or equal to 8 minutes, “B” indicates a Tp of between 8 minutes and 12 minutes (inclusive), “C” indicates a Tp of between 12 minutes and 25 minutes (inclusive), and “D” indicates a Tp of greater than 25 minutes or no amplification detected (No Call).

TABLE 4
Time to Positive (Dye Detection)

| | | Primer | 10 | Neg. | |
|---|---|---|---|---|---|
| Organism | Target | Set # | CFU/mL | Urine | NTC |
| NG | 23S | Set-59 | A | C | C |
| NG | 23S | Set-60 | A | D | B |
| NG | 23S | Set-61 | A | D | D |
| NG | 23S | Set-62 | B | D | D |
| NG | 23S | Set-63 | B | C | C |
| NG | 16S | Set-77 | D | D | D |
| NG | rsmB | Set-80 | A | D | D |
| NG | rsmB | Set-81 | A | D | C |
| NG | rsmB | Set-82 | D | D | D |
| NG | rsmB | Set-83 | C | D | C |
| NG | rsmB | Set-84 | C | C | B |
| NG | rsmB | Set-85 | A | D | D |

### Example 3: Cross-Reactivity Using Dye Detection

Amplification reactions containing some of the above primers sets for detection of C. and the intercalating dye resulted in the detection of an amplification product when using water or negative urine extraction or the DNA of closely related specie such as C. pneumoniae or C. psittaci as templates at frequencies ranging between 0% to 75% of the time (Table 5), within variable intervals of our cut off window for the assay time. Results are classified by the time to position: A having Tp in less or equal to 8 minutes, B having Tp between 8 minutes and 12 minutes (inclusive), C having Tp greater than 12 minutes, and D having no amplification detected.

TABLE 5
Cross Reactivity - Dye Detection

| | | Primer | Negative Urine | | NTC | | C. pneumoniae | | C. psittaci | |
|---|---|---|---|---|---|---|---|---|---|---|
| Organism | Target | Set # | extraction | | (water) | | DNA | | DNA | |
| CT | 23S | Set-1 | C | 2 of 4 | C | 1 of 2 | C | 2 of 2 | C | 2 of 2 |
| CT | 23S | Set-2 | D | 0 of 4 | D | 0 of 2 | C | 1 of 2 | D | 0 of 2 |
| CT | 23S | Set-3 | D | 0 of 4 | B | 1 of 2 | C | 1 of 2 | C | 2 of 2 |
| CT | 23S | Set-4 | C | 1 of 4 | D | 0 of 2 | D | 0 of 2 | C | 1 of 2 |
| CT | 23S | Set-5 | C | 1 of 4 | D | 0 of 2 | D | 0 of 2 | C | 1 of 2 |
| CT | 23S | Set-6 | C | 3 of 4 | C | 1 of 2 | C | 2 of 2 | C | 1 of 2 |

A subset of the primer sets specific for detection of N. gonorrhoeae described in Example 2 were additionally tested for specificity by comparing reactions with 10⁹ copies of N. gonorrhoeae gDNA template (NG) to reactions with 10⁹ copies of gDNA from closely related Neisseria species, Neisseria meningitides (NM), Neisseria lactamica (NL), and Neisseria sicca (NS). When the amplification reactions were performed as described in Example 2, each of the primer sets tested had significant cross-reactivity against additional Neisseria species (Table 6). As expected, due to the high concentration of template, the LAMP reactions occur very quickly. Results are classified by the time to positive (Tp) from reaction initiation as follows: “A” indicates a Tp of less than or equal to 5 minutes, “B” indicates a Tp of between 5 minutes and 8 minutes (inclusive), “C” indicates a Tp of between 8 minutes and 15 minutes (inclusive), and “D” indicates a Tp of greater than 26 minutes or no amplification detected. Each of the primer sets showed cross reactivity with several of the closely related Neisseria species.

TABLE 6
Cross-Reactivity (Dye Detection) for NG primer sets

| | | Primer | | | | | Neg. | |
|---|---|---|---|---|---|---|---|---|
| Organism | Target | Set # | NG | NM | NL | NS | Urine | NTC |
| NG | 23S | Set-61 | A | B | C | B | D | D |
| NG | 23S | Set-62 | A | A | B | A | D | D |
| NG | rsmB | Set-80 | A | D | A | B | D | D |
| NG | rsmB | Set-85 | A | D | C | B | D | D |

### Example 4: Beacon Design Location Effect on Assay Kinetics

For added specificity molecular beacons were designed along these primers sets to make sure only signal from the CT or NG target is detected (sequences listed in Table 7). Each molecular beacon probe was designed with 5′ fluorophore/3′ quencher modifications (6-Carboxyfluorescein (FAM) and Black Hole Quencher 1 (BHQ1)) included to provide target-specific fluorescent detection.

TABLE 7
Probe Sequences

| | | | Beacon | | | | |
|---|---|---|---|---|---|---|---|
| Organism | Target | Beacon ID | # | Fluor | Quench | Sequence (5′ to 3′) | NEW SEQ ID |
| CT | 23S | Bs1 | MB1 | FAM | BHQ1 | CGCGATCAGGACTCCTAGTTGAACACATCTGGATCGCG | SEQ ID NO: 180 |
| CT | 23S | Bs14 | MB2 | FAM | BHQ1 | CGTCCAGGACTCCTAGTTGAACACATCTGGACG | SEQ ID NO: 181 |
| CT | 23S | Bs4bis | MB3 | FAM | BHQ1 | CGCGACTCAGGACTCCTAGTTGAACACATCTGGAGTCG | SEQ ID NO: 182 |
| | | | | | | CG | |
| CT | 23S | Bs6bis | MB4 | FAM | BHQ1 | CGCGAGTAGGATTGAGGATAAAGGATCAGGACTCGCG | SEQ ID NO: 183 |
| CT | 23S | Bs8bis | MB5 | FAM | BHQ1 | CGCGCACATCTGGAAAGATGGATGATACAGGGTGCGCG | SEQ ID NO: 184 |
| CT | 23S | Bs5 | MB6 | FAM | BHQ1 | CGCGATCCGGATAAAGGATCAGGACTCCTAGTTGGGAT | SEQ ID NO: 185 |
| | | | | | | CGCG | |
| CT | 23S | Bs12 | MB7 | FAM | BHQ1 | CGCGATCAGACCGACCTCAACACCTGAGATCGCG | SEQ ID NO: 186 |
| CT | 23S | Bs13 | MB8 | FAM | BHQ1 | CGCAGTGAGAGAAAGACCGACCTCAACACTGCG | SEQ ID NO: 187 |
| CT | 23S | Bs10 | MB9 | FAM | BHQ1 | CGCGATCCTGTGTAGCGGCGAGCGAAAGATCGCG | SEQ ID NO: 188 |
| CT | 23S | B inner | MB10 | FAM | BHQ1 | CGCGATCAGATCCATGGCATAAGTAACGGATCGCG | SEQ ID NO: 189 |
| | | n1 | | | | | |
| CT | 23S | B inner | MB11 | FAM | BHQ1 | CGCGATCGGTGAAGATCCATGGCATAAGTAACGCGATC | SEQ ID NO: 190 |
| | | n2 | | | | GCG | |
| CT | 23S | Bn3 | MB12 | FAM | BHQ1 | CGCGATCCATGGCATAAGTAACGATAAAGGGAGTGAGG | SEQ ID NO: 191 |
| | | | | | | ATCGCG | |
| CT | 23S | Bn4 | MB13 | FAM | BHQ1 | CGCGATCATGACGGAGTAAGTTAAGCACGCGATCGCG | SEQ ID NO: 192 |
| CT | 23S | Bs8 | MB14 | FAM | BHQ1 | CGCGATGTCTGGAAAGATGGATGATACAGCATCGCG | SEQ ID NO: 193 |
| CT | 23S | Bs6 | MB15 | FAM | BHQ1 | CGCGATCTGAGGATAAAGGATCAGGACTCGATCGCG | SEQ ID NO: 194 |
| CT | 23S | Bs9 | MB16 | FAM | BHQ1 | CGCGATCCCTGTGTAGCGGCGAGCGAGATCGCG | SEQ ID NO: 195 |
| CT | 23S | Bn1 (F | MB17 | FAM | BHQ1 | CGCGATCGCAAAAGGCACGCCGTCAAGATCGCG | SEQ ID NO: 196 |
| | | loop) | | | | | |
| CT | 23S | Bs18 | MB18 | FAM | BHQ1 | CGGCTCGGGGTTGTAGGATTGAGGATACGAGCCG | SEQ ID NO: 197 |
| CT | 23S | Bs19 | MB19 | FAM | BHQ1 | CCGGAGCCTACAACCCCGAGCCTTATCAGCTCCGG | SEQ ID NO: 198 |
| | | antisense | | | | | |
| CT | 23S | Bs20 | MB20 | FAM | BHQ1 | CGCGCAGCTCGGTTTAGGCTATTCCCCTGCGCG | SEQ ID NO: 199 |
| | | antisense | | | | | |
| CT | 23S | Bs21 | MB21 | FAM | BHQ1 | CGCGGTCTCTCCTTTCGTCTACGGGACCGCG | SEQ ID NO: 200 |
| | | antisense | | | | | |
| CT | 16S | Bs1 | MB22 | FAM | BHQ1 | CGCGATCATCCGAGTAACGTTAAAGAAGGGGATCGCG | SEQ ID NO: 201 |
| CT | 16S | Bs4 | MB23 | FAM | BHQ1 | CGCGATCTGGCGATATTTGGGCATCCGAGATCGCG | SEQ ID NO: 202 |
| NG | 23S | Bs1 | MB24 | FAM | BHQ1 | CGCGATCCACGAGCACTCTTGCCAACACGATCGCG | SEQ ID NO: 203 |
| NG | 23S | Bs3 | MB25 | FAM | BHQ1 | CGCGATCGGAGCACTCTTGCCAACACGAAAGCGATCGC | SEQ ID NO: 204 |
| | | | | | | G | |
| NG | 23S | Bs7 | MB26 | FAM | BHQ1 | CGCGATCAGCACTCTGCCAACACGAAAGATCGCG | SEQ ID NO: 205 |
| NG | 23S | Bn2 inner | MB27 | FAM | BHQ1 | CGCGATCCGTCCTGCGCGGAAGATGTAACGGGATCGCG | SEQ ID NO: 206 |
| NG | 23S | Bn1 inner | MB28 | FAM | BHQ1 | CGCGATCCTGCGCGGAAGATGTAACGATCGCG | SEQ ID NO: 207 |
| NG | 23S | Bs2 | MB29 | FAM | BHQ1 | CGCGATCGCACGAGCACTCTTGCCCGATCGCG | SEQ ID NO: 208 |
| NG | 23S | Bs4 | MB30 | FAM | BHQ1 | CGCGATCACTTGTTTATTAAAAACACGGATCGCG | SEQ ID NO: 209 |
| NG | 23S | Bs5 | MB31 | FAM | BHQ1 | CGCGACCCGACTTGTTTATTAAAAACACGAGCACGGTC | SEQ ID NO: 210 |
| | | | | | | GCG | |
| NG | 23S | Bs6 | MB32 | FAM | BHQ1 | CGCGACCCCGACTTGTTTATTAAAAACACGAGCACGGG | SEQ ID NO: 211 |
| | | | | | | TCGCG | |
| NG | 23S | Bn1 loop | MB33 | FAM | BHQ1 | CGCGATCAGCAGCCATCATTTAAAGAAAGGATCGCG | SEQ ID NO: 212 |
| NG | rsmB | Bs1 | MB34 | FAM | BHQ1 | CAGGCCGGTTTTGCCGAAGGACTGGTGTCGGCCTG | SEQ ID NO: 213 |
| NG | rsmB | Bs3 | MB35 | FAM | BHQ1 | CGCGAAGGACTGGTGTCGGTACAGGACTTCGCG | SEQ ID NO: 214 |
| NG | rsmB | Bs2 | MB36 | FAM | BHQ1 | CTAGCGAAGGACTGGTGTCGCTAG | SEQ ID NO: 215 |
| NG | rsmB | Bs4 | MB37 | FAM | BHQ1 | CGCGATGTTTGCCGAAGGACTGGTGTCATCGCG | SEQ ID NO: 216 |
| NG | rpIF | Bs1 | MB38 | FAM | BHQ1 | CGCGATCGAAGAAATTACAATTGATGGGCGTGATCGCG | SEQ ID NO: 217 |
| NG | rpIF | Bs2 | MB39 | FAM | BHQ1 | CGCGATCGAAGAAATTACAATTGATAGGCGGATCGCG | SEQ ID NO: 218 |

### Example 5: Detection of 23S CT Using Oligonucleotide Probes That Bind to the DS Region of an Amplicon

We tested combinations of primer sets and oligonucleotide probes to generate amplicons that have probe target sequences in the DS region, the Loop region, or both (DS/Loop) in the amplicon, to determine whether we can detect amplicons using oligonucleotide probes that bind, at least partially to a DS region of an amplicon. In this example, the target nucleic acid is 23S from C. trachomatis (CT).

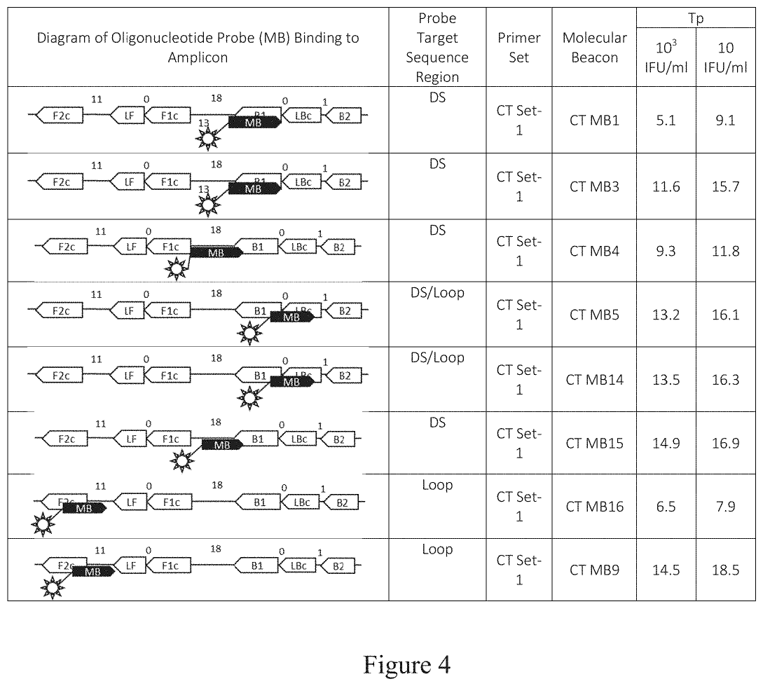

From A negative urine matrix was spiked with titred C. trachomatis (serially diluted in PBS, Zeptometrix CN#0801775) at two different concentrations (10³ IFU/mL and 10 IFU/mL). Nucleic acids were extracted using standard extraction methods and the sample was amplified using a LAMP primer set (Sets described in Table 2, SEQ ID NOs) and one of the molecular beacons (Table 7) was used for the detection of the amplified product. In this example, a 25 μL reaction contained 1× Isothermal Amplification Buffer or Thermopol DF buffer (New England Biolabs) supplemented with 4.8 mM or 6 mM MgCl₂, 1.4 mM or 1.6 mM dNTP, 200 nM YO-PRO-1 dye (Life Technologies), primers (2 μM of F3 and B3, if present; 1.6 μM or 2 μM of FIP and BIP; 8 μM of LF and LB, if present), 8 or 12 Units of Bst2 polymerase (New England Biolabs), 7.5 Units RTx Warmstart (reverse transcriptase; New England Biolabs), and the extracted nucleic acid (as template) or water (as no template control). The reactions were incubated at 63° C. or 65° C. and kinetics were monitored using a Roche real-time Lightcycler96 (Roche). FIG. 4 shows a diagram of the amplicon generated by each primer set and the location of oligonucleotide probe binding to the amplicon (i.e., the probe target sequence). FIG. 4 also reports the time from LAMP initiation to signal detection for each primer-probe combination at high (10³ IFU/mL) and low (10 IFU/mL) target nucleic acid concentrations.

As shown in FIG. 4, molecular beacons that bind to the DS region, or within a portion of the DS region and the Loop region (DS/Loop), can be used to detect the presence of an amplicon in a sample with both a high and a low concentration of target nucleic acid. The time to detection for each LAMP assay using molecular beacons that bind to at least a portion of the DS region of the amplicon is comparable to LAMP assays using oligonucleotide probes that do not bind to any portion of the DS region (Loop).

### Example 6: Detection Times for C. trachomatis and N gonorrhoeae Target Nucleic Acids Using Probes Binding to DS and/or Loop, Regions of a LAMP Amplicon

A negative urine matrix was spiked with titred C. trachomatis or N gonorrhoeae (serially diluted in PBS, Zeptometrix CN#0801775) at two different concentrations (10³ IFU/mL and 10 IFU/mL). Nucleic acids were extracted using standard extraction methods and the sample was amplified using a LAMP primer set (Sets described in Table 2, SEQ ID NOs) and one of the molecular beacons (Table 7) was used for the detection of the amplified product. In this example, a 25 μL reaction contained 1× Isothermal Amplification Buffer or Thermopol DF buffer (New England Biolabs) supplemented with 4.8 mM or 6 mM MgCl₂, 1.4 mM or 1.6 mM dNTP, 200 nM YO-PRO-1 dye (Life Technologies), primers (2 μM of F3 and B3, if present; 1.6 μM or 2 μM of FIP and BIP; 8 μM of LF and LB, if present), 8 or 12 Units of Bst2 polymerase (New England Biolabs), 7.5 Units RTx Warmstart (reverse transcriptase; New England Biolabs), and the extracted nucleic acid (as template) or water (as no template control). The reactions were incubated at 63° C. or 65° C. and kinetics were monitored using a Roche real-time Lightcycler96 (Roche).

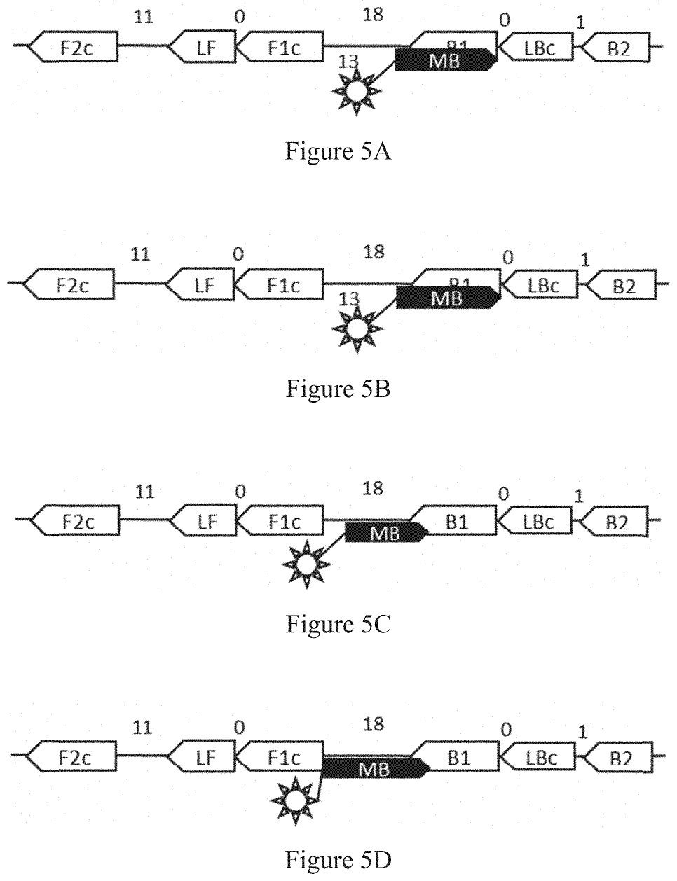

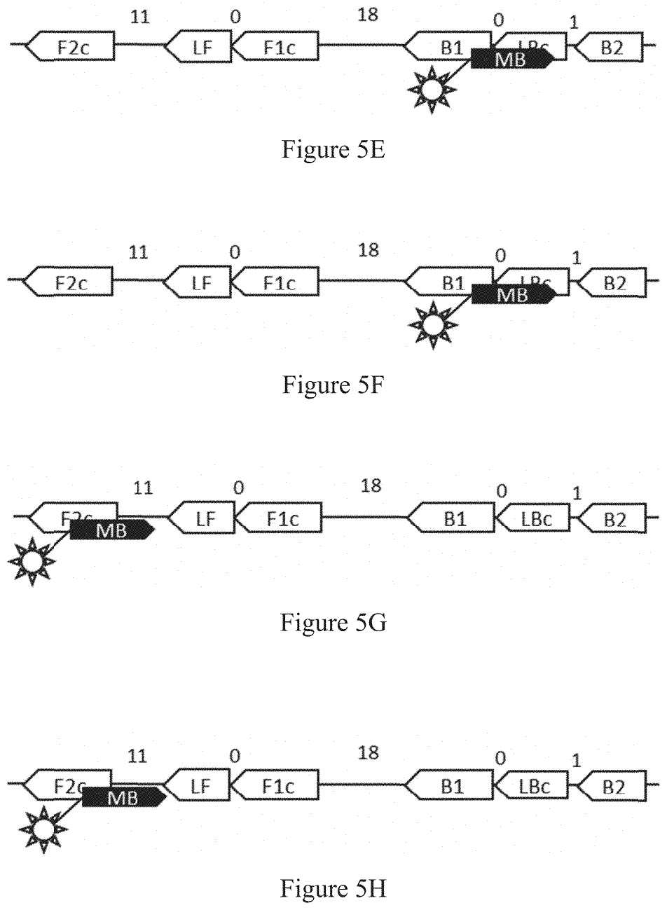

Table 8 provides details on each LAMP primer set and oligonucleotide probe combination used for nucleic acid target detection. The probe binding region (DS, Loop, or DS/Loop) is indicated, with diagrams showing the binding location of the oligonucleotide probes to amplicons generated by the paired set for selected combinations in FIGS. 5A-32B, as indicated in Table 8. The time to positive for each primer-probe combination is also reported in Table 8 for samples run target nucleic acid from samples containing 10³ IFU/mL or 10 IFU/mL. Results are classified by the time to positive: A having Tp in less or equal to 10 minutes, B having Tp between 10 minutes and 15 minutes (inclusive), C having Tp greater that 15 minutes. NT indicates that this combination was not tested.

The results shown in Table 8 indicate successful design and use of oligonucleotide probes that bind to at least a portion of the DS region of a LAMP amplicon for detection of the presence of absence of several different types of target nucleic acid, including RNA target nucleic acids using RT-LAMP.

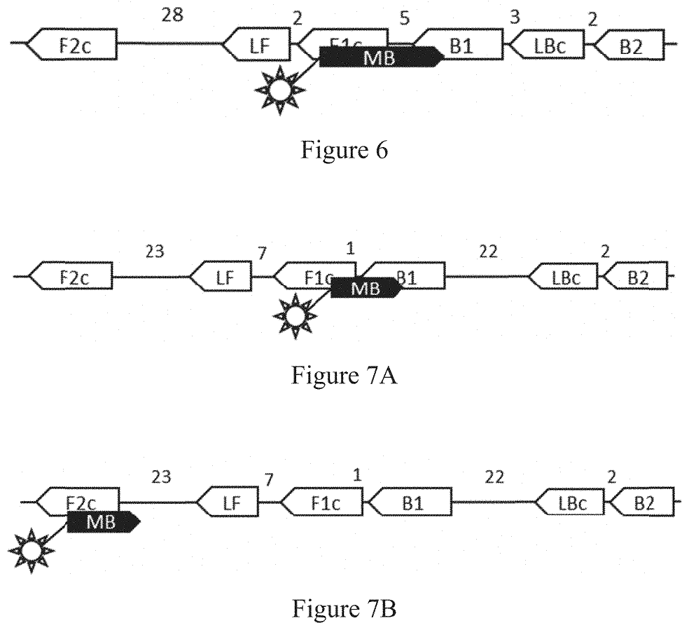

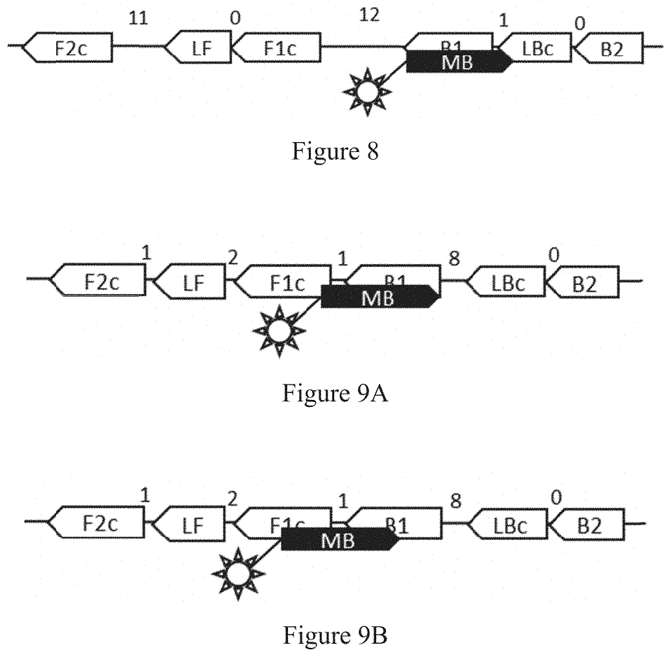

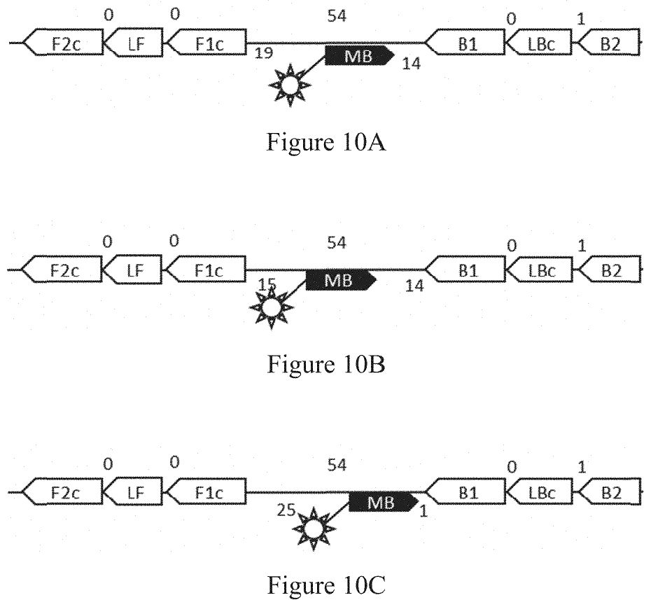

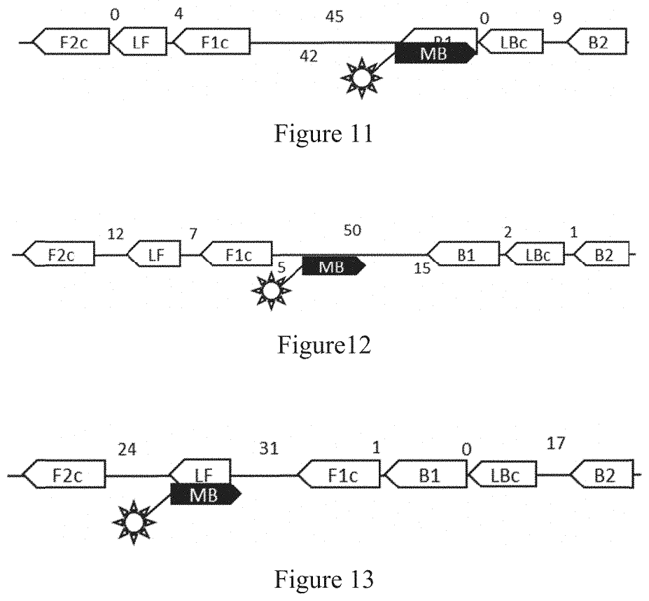

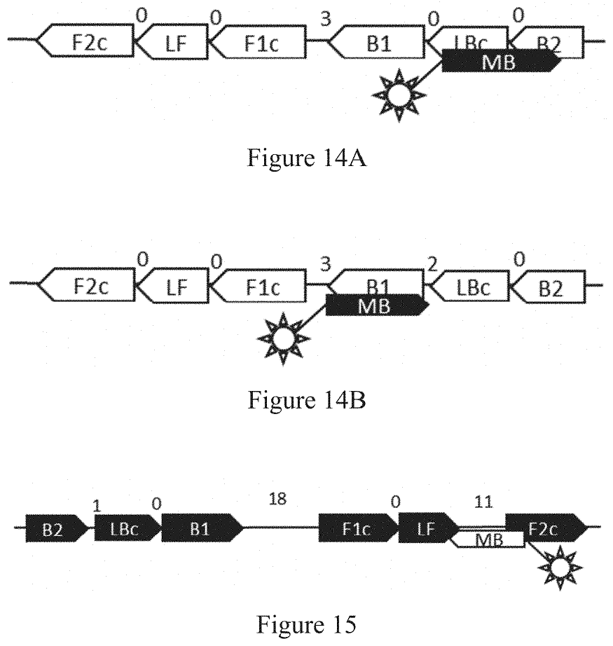

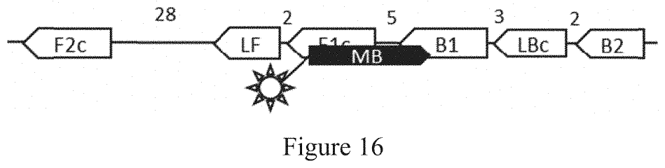

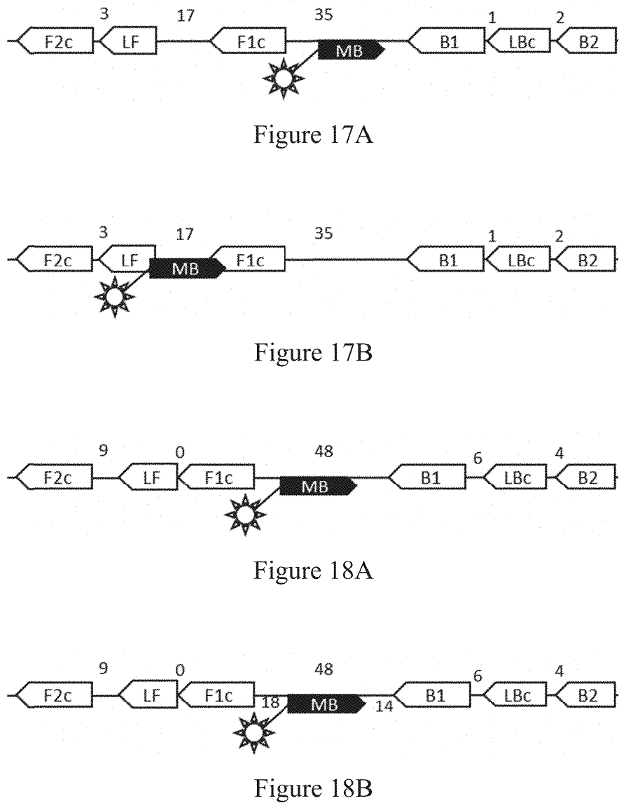

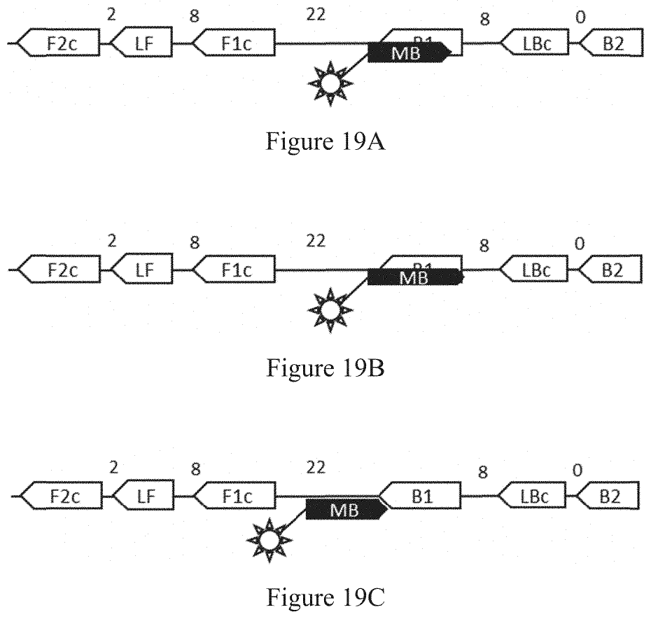

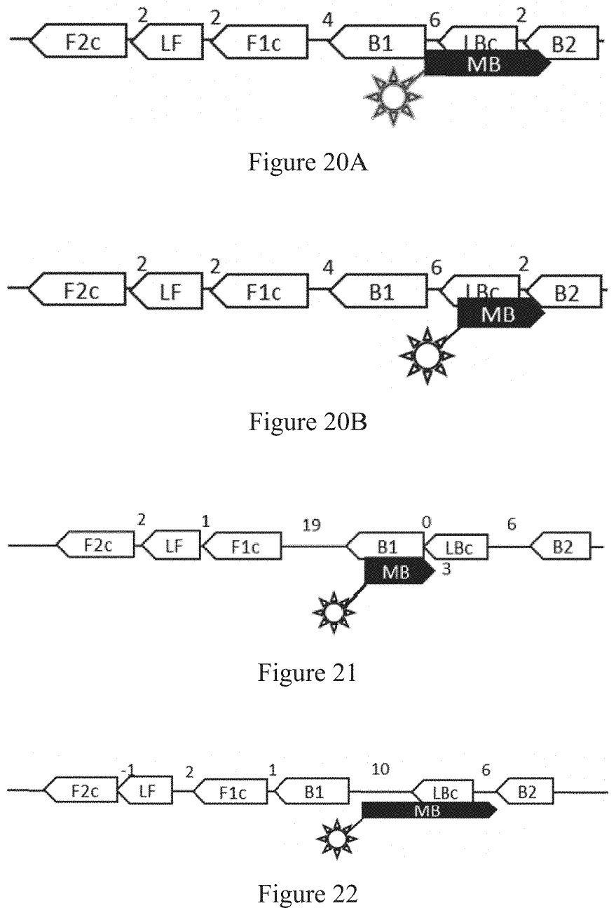

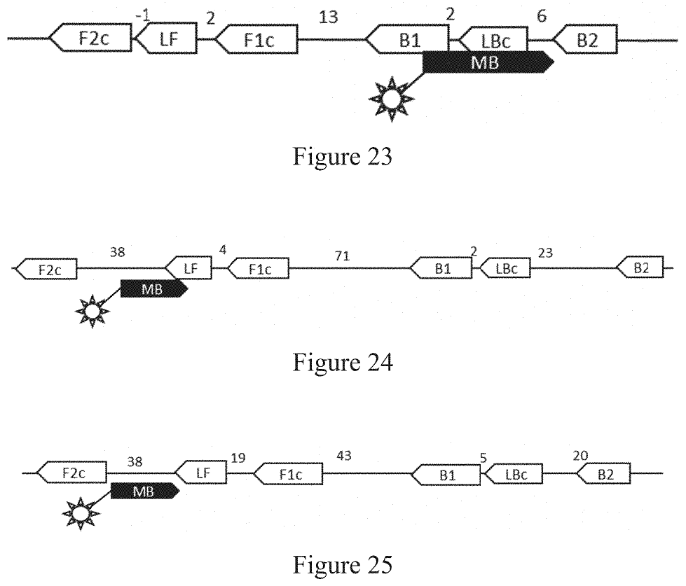

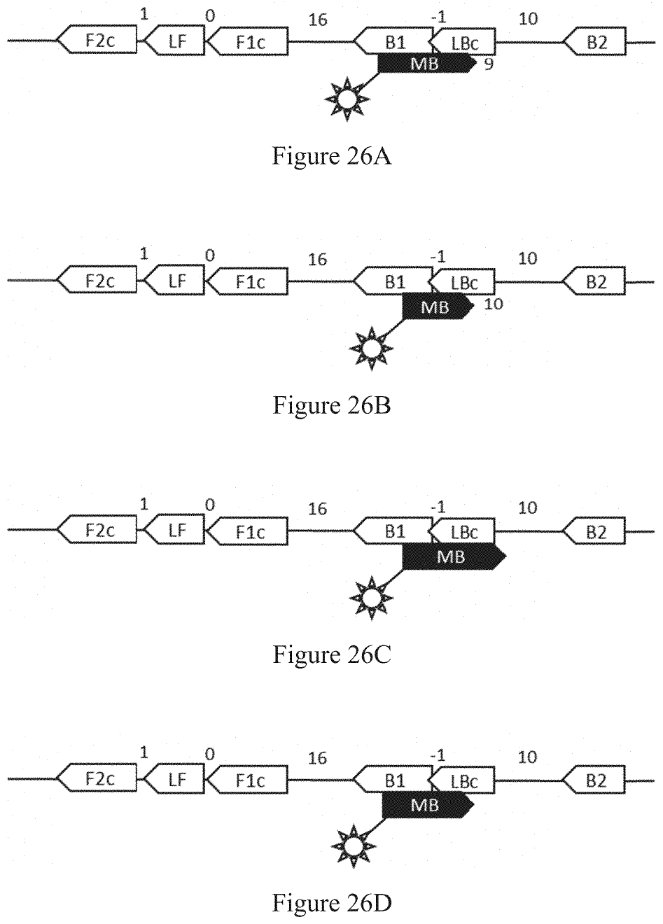

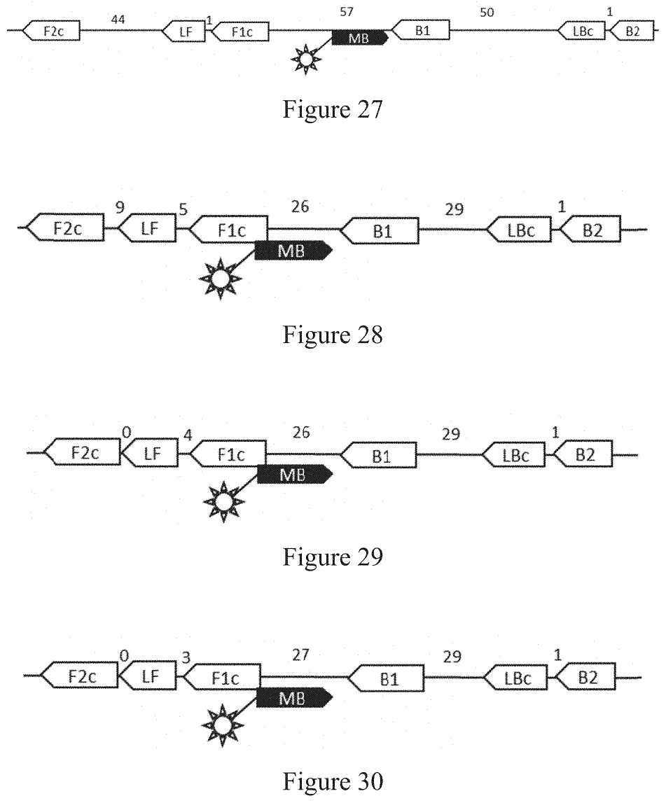

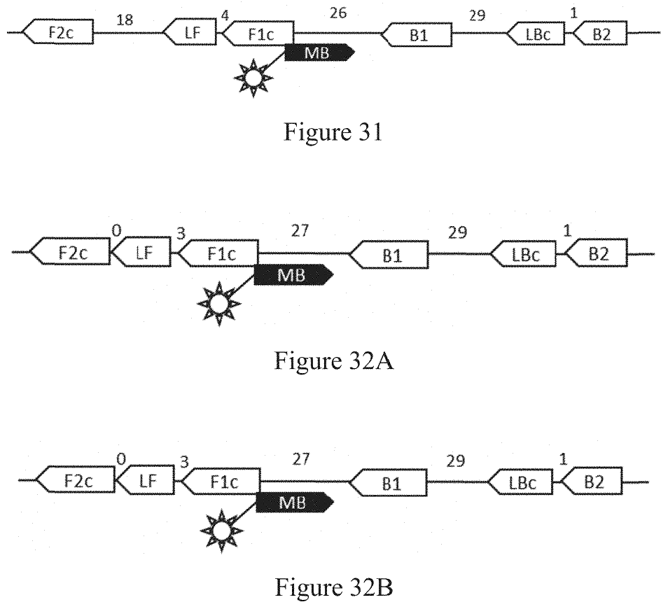

TABLE 8
Time to Positive Probe Detection

| | | | | | | Probe | | |
|---|---|---|---|---|---|---|---|---|
| | | | Primer | Beacon | | Binding | 10³ | 10 |
| Organism | Target | ID | Set # | # | Diagram | Region | IFU/mL | IFU/mL |
| CT | 23S | Ps6-Bs1 | Set-1 | MB1 | FIG. 5A | DS | A | A |
| | | | | | | | A | B |
| | | | | | | | A | B |
| CT | 23S | Ps6-Bs4bis | Set-1 | MB3 | FIG. 5B | DS | B | C |
| CT | 23S | Ps6-Bs6 | Set-1 | MB15 | FIG. 5C | DS | B | C |
| CT | 23S | Ps6-Bs6bis | Set-1 | MB4 | FIG. 5D | DS | A | B |
| CT | 23S | Ps6-Bs8-B | Set-1 | MB14 | FIG. 5E | DS/Loop | B | C |
| | | loop | | | | | | |
| CT | 23S | Ps6-Bs8bis-B | Set-1 | MB5 | FIG. 5F | DS/Loop | B | B |
| | | loop | | | | | | |
| CT | 23S | Ps6-Bs9 F | Set-1 | MB16 | FIG. 5G | Loop | A | A |
| | | loop | | | | | | |
| CT | 23S | Ps6-Bs10 | Set-1 | MB9 | FIG. 5H | Loop | C | C |
| | | | | | | | B | C |
| CT | 23S | Ps7-Bs1 | Set-2/ | MB1 | FIG. 6 | DS | B | B |
| | | | Set-11 | | | | A | A |
| CT | 23S | Ps8-Bs1 | Set-3 | MB1 | FIG. 7A | DS | A | B |
| | | | | | | | A | B |
| CT | 23S | Ps8-Bs8bis | Set-3 | MB5 | | DS/Loop | B | B |
| CT | 23S | Ps8-Bs9 | Set-3 | MB16 | FIG. 7B | Loop | B | B |
| CT | 23S | Ps11-Bs1 | Set-4 | MB1 | FIG. 8 | DS/Loop | B | C |
| CT | 23S | Ps11-Bs5 | Set-4 | MB6 | | DS | C | C |
| CT | 23S | Ps12-Bs12 | Set-5/ | MB7 | FIG. 9A | DS | A | B |
| | | | Set-12 | | | | A | NT |
| CT | 23S | Ps12-Bs13 | Set-5/ | MB8 | FIG. 9B | DS | A | B |
| | | | Set-12 | | | | A | B |
| CT | 23S | Ps13-Bs13 | Set-6 | MB8 | | DS | B | B |
| CT | 23S | Pn2-Bn1- | Set-7 | MB10 | FIG. 10A | DS | B | NT |
| | | inner | | | | | | |
| CT | 23S | Pn2-Bn2- | Set-7 | MB11 | FIG. 10B | DS | C | C |
| | | inner | | | | | | |
| CT | 23S | Pn2-Bn3- | Set-7 | MB12 | FIG. 10C | DS | A | B |
| | | inner | | | | | | |
| CT | 23S | Pn3-Bn4- | Set-8 | MB13 | FIG. 11 | DS | B | NT |
| | | inner | | | | | | |
| CT | 23S | Ps1-Bs1 | Set-9 | MB1 | FIG. 12 | DS | NT | C |
| CT | 23S | Ps2(1)-Bs1 | Set-10 | MB1 | FIG. 13 | Loop | C | C |
| CT | 23S | Ps7-Bs1 | Set-11 | MB1 | | DS | A | A |
| CT | 23S | Ps14-Bs14 | Set-13 | MB2 | FIG. 14A | Loop | A | A |
| CT | 23S | Ps14-Bs18 | Set-13 | MB18 | FIG. 14B | DS | A | B |
| CT | 23S | Pn1-Bn1-F | Set-14 | MB17 | FIG. 15 | Loop | | |
| | | loop | | | | | | |
| | | (antisense) | | | | | | |
| CT | 16S | Ps1bis-Bs1 | Set-47 | MB22 | | DS | B | C |
| CT | 16S | Ps6-Bs1 | Set-48 | MB22 | | DS | B | C |
| CT | 16S | Ps7-Bs4 | Set-49 | MB23 | FIG. 16 | DS | B | NT |
| NG | 23S | Pn2-Bn1 | Set-59 | MB28 | | DS | A | B |
| | | inner | | | | | | |
| NG | 23S | Pn2-Bn2 | Set-59 | MB27 | FIG. 17A | DS | A | B |
| | | inner | | | | | | |
| NG | 23S | Ps1-Bs1 | Set-60 | MB24 | FIG. 18A | DS | B | C |
| NG | 23S | Ps1-Bs2 | Set-60 | MB29 | FIG. 18B | DS | B | NT |
| NG | 23S | Ps1-Bs3 | Set-60 | MB25 | | DS | B | NT |
| NG | 23S | Ps1-Bs7 | Set-60 | MB26 | | DS | A | B |
| NG | 23S | Ps2-Bs1 | Set-61 | MB24 | FIG. 19A | DS | C | C |
| NG | 23S | Ps2-Bs2 | Set-61 | MB29 | FIG. 19B | DS | C | NT |
| NG | 23S | Ps2-Bs3 | Set-61 | MB25 | | DS | C | NT |
| NG | 23S | Ps2-Bs4 | Set-61 | MB30 | FIG. 19C | DS | NT | NT |
| NG | rsmB | Ps1-Bs1 | Set-80 | MB34 | FIG. 20A | Loop | A | A |
| NG | rsmB | Ps1-Bs2 | Set-80 | MB36 | FIG. 20B | Loop | B | C |
| NG | rsmB | Ps2-Bs2 | Set-81 | MB36 | FIG. 21 | DS/Loop | B | C |
| NG | rsmB | Ps3-Bs1 | Set-82 | MB34 | FIG. 22 | Loop | C | C |
| NG | rsmB | Ps3bis-Bs1 | Set-91 | MB34 | FIG. 23 | DS/Loop | C | C |
| NG | rsmB | Ps4-Bs1 | Set-83 | MB34 | FIG. 24 | Loop | B | C |
| NG | rsmB | Ps5-Bs1 | Set-84 | MB34 | FIG. 25 | Loop | B | C |
| NG | rsmB | Ps6-Bs1 | Set-85 | MB34 | FIG. 26A | DS/Loop | NT | A |
| NG | rsmB | Ps6-Bs2 | Set-85 | MB36 | FIG. 26B | DS/Loop | NT | B |
| NG | rsmB | Ps6-Bs6 | Set-85 | MB35 | FIG. 26C | DS/Loop | NT | B |
| NG | rsmB | Ps6-Bs6 | Set-85 | MB37 | FIG. 26D | DS/Loop | NT | A |
| NG | rplF | Ps1-Bs1 | Set-115 | MB38 | FIG. 27 | DS | C¹ | C² |
| NG | rplF | Ps2-Bs1 | Set-116 | MB38 | FIG. 28 | DS | C | C |
| NG | rplF | Ps3-Bs1 | Set-117 | MB38 | FIG. 29 | DS | B | B |
| NG | rplF | Ps4-Bs1 | Set-118 | MB38 | FIG. 30 | DS | B | B |
| NG | rplF | Ps5-Bs1 | Set-119 | MB38 | FIG. 31 | DS | C | C |
| NG | rplF | Ps6-Bs1 | Set-120 | MB38 | FIG. 32A | DS | C | C |
| NG | rplF | Ps6-Bs2 | Set-120 | MB39 | FIG. 32B | DS | C | C |

¹100 CFU/mL
²2 CFU/mL

Use of Molecular Beacons as compared to dye for detection resulted in a slight increase in reaction Tp, however the significant enhancement in assay specificity provided a reasonable tradeoff, no amplification was observed in the negative urine extract or water sample or DNA from a close related species within the testing period of 45 min.

### Example 7: TTP Detection Based on Genomic DNA Concentration

Chlamydia trachomatis gDNA (ATCC CN#VR-885D) was diluted using TE buffer at two different concentrations (10⁵ genome copies/μl and 10³ genome N. gonorrhoeae gDNA was diluted using TE buffer to known concentrations. The samples were amplified using a LAMP primer set (Sets described in Table 2, SEQ ID NOs) and one of the molecular beacons (Table 7) was used for the detection of the amplified product. In this example, a 25 μL reaction contained 1× Isothermal Amplification Buffer or Thermopol DF buffer (New England Biolabs) supplemented with 4.8 mM or 6 mM MgCl₂, 1.4 mM or 1.6 mM dNTP, 200 nM molecular beacon(Sigma-Aldrich), primers (0.2 μM of F3 and B3, if present; 1.6 μM or 2 μM of FIP and BIP; 0.8 μM of LF and LB, if present), 8 or 12 Units of Bst2 polymerase (New England Biolabs), 7.5 Units RTx Warmstart (reverse transcriptase; New England Biolabs), and the gDNA dilutions (as template) or water (as no template control). The reactions were incubated at 63° C. or 65° C. and kinetics were monitored using a Roche real-time Lightcycler96 (Roche). The time to positive for each primer-probe combination is reported in Table 9. Results are classified by the time to positive: A having Tp in less or equal to 10 minutes, B having Tp between 10 minutes and 15 minutes (inclusive), C having Tp greater that 15 minutes. NT indicates that this combination was not tested.

TABLE 9
Time to Positive Probe Detection based on genomic DNA concentration

| | | | | | | Genomic DNA |
|---|---|---|---|---|---|---|
| | | | | Probe | 5 × 10⁵ | concentration |
| | | Primer | Beacon | Binding | genome | (genome |
| Organism | Target | Set # | # | Region | copies/μL | copies/μL) |
| CT | 23S | Set-1 | MB19 | DS | A | 5 × 10⁵ |
| | | | | | A | 5 × 10³ |
| CT | 23S | Set-1 | MB20 | DS/Loop | A | 5 × 10⁵ |
| | | | | | A | 5 × 10³ |
| CT | 23S | Set-1 | MB21 | Loop | A | 5 × 10⁵ |
| | | | | | A | 5 × 10³ |
| NG | 23S | Set-59 | MB33 | DS/Loop | C | 1.43 × 10⁵ |
| NG | 23S | Set-60 | MB24 | DS | B | 7 × 10⁶ |
| NG | 23S | Set-60 | MB29 | DS | B | 7 × 10⁶ |
| NG | 23S | Set-60 | MB26 | DS | A | 2 × 10⁵ |
| NG | 23S | Set-61 | MB24 | DS | C | 2 × 10⁵ |
| NG | 23S | Set-61 | MB30 | DS | C | 2 × 10⁵ |
| NG | 23S | Set-61 | MB21 | DS | C | 2 × 10⁵ |
| NG | 23S | Set-61 | MB32 | DS | C | 2 × 10⁵ |
| NG | rsmB | Set-80 | MB34 | Loop | A | 6 × 10⁵ |
| NG | rsmB | Set-81 | MB36 | DS/Loop | C | 6 × 10⁵ |
| NG | rsmB | Set-82 | MB34 | Loop | C | 6 × 10⁵ |
| NG | rsmB | Set-91 | MB34 | DS/Loop | C | 6 × 10⁵ |
| NG | rsmB | Set-83 | MB34 | Loop | B | 6 × 10⁵ |
| NG | rsmB | Set-85 | MB34 | DS/Loop | A | 6 × 10⁵ |
| NG | rsmB | Set-85 | MB36 | DS/Loop | A | 6 × 10⁵ |
| NG | rsmB | Set-85 | MB35 | DS/Loop | A | 6 × 10⁵ |
| NG | rsmB | Set-85 | MB37 | DS/Loop | A | 6 × 10⁵ |

Use of Molecular Beacons for detection resulted in a slight increase in reaction Tp, however the significant enhancement in assay specificity provided a reasonable tradeoff, no amplification was observed in the negative urine extract or water sample or DNA from a close related species within the testing period of 45 min.

### Example 8: Specificity Testing

A negative urine matrix was spiked with titred C. trachomatis or with organisms commonly associated with urine infections at high loads (E. coli, C. albicans, S. aureus, P. mirabilis), sexually transmitted infections (Chlamydia trachomatis), or species closely related to C. trachomatis (C. pneumonia or C. psitascii). Bacterial stocks were serially diluted in PBS before addition to the urine matrix at the desired concentration. Corresponding extracted nucleic acids or DNAs of the test species were used as templates in RT-LAMP reactions containing the LAMP primers (Set-1) and the molecular beacon probe MB2. Reaction conditions are equivalent to those described above in Example 6. The designed primers and probe resulted in no amplification with the non-C. trachomatis species tested.

This example shows that the designed CT23S assay and its reaction formulation is highly specific and does not cross react with sequences of organisms commonly found in urine and vaginal clinical samples.

25 μl total volume reactions using 10⁹ copies of gDNA of N. gonorrhoeae or closely related Neisseria species. Use of Molecular Beacons for detection resulted in a slight increase in reaction Tp, however the significant enhancement in assay specificity provided a reasonable tradeoff (Table 10). Results are classified by the time to positive (Tp) from reaction initiation as follows: “A” indicates a Tp of less than or equal to 9 minutes, “B” indicates a Tp of between 9 minutes and 15 minutes (inclusive), and “C” indicates a Tp of greater than 15 minutes or no amplification detected (No Call). An asterisk indicates an amplification curve with a shallow slope combined with a significantly reduced maximal fluorescence relative to N. gonorrhoeae reactions (i.e., no greater than 5%).

TABLE 10
Cross-Reactivity (Molecular Beacon)

| | | Primer | Beacon | Tp | Tp | Tp | Tp | Tp |
|---|---|---|---|---|---|---|---|---|
| Organism | Target | Set # | # | NG | NM | NL | NS | NTC |
| NG | rsmB | Set-80 | MB34 | A | C | C* | C | C* |
| NG | rsmB | Set-85 | MB35 | A | C | C* | C* | C* |
| NG | 23S | Set-62 | MB24 | B | C | B | C | C |
| NG | 23S | Set-62 | MB25 | B | C | B | C | C |
| NG | 23S | Set-62 | MB26 | A | C | A | C | C |

Potentially cross reacting organisms were tested and included common urinary tract and/or vaginal microbial colonizers and the closest N. gonorrhoeae phylogenetic relatives. Template input for amplification reactions was either from purified genomic DNA (gDNA) purchased from Zeptometrix at known concentrations or nucleic acids extracted from live bacterial or yeast cells. Except where indicated (*), live titred cells or known concentrations of genomic DNA were used as input for amplification reactions. In instances marked with an asterisk, where titred material and/or known concentrations were not available, template concentration was approximated based on RTqPCR standard curve Cq's. The assay was performed using Primer Set-80 and MB34 with RT-LAMP as described above. Positive calls were determined using the accompanying real time cycler standard analysis packages (Roche LightCycler 96 Software version 1.1.0.1320 or Bio-Rad CFX Manager Software version 3.1.1517.0823).

TABLE 11
Assay Specificity

| Organism | Nucleic acid source | Concentration | % positive |
|---|---|---|---|
| Neisseria gonorrhoeae | extracted from cells | 1 × 10² CFU × mL⁻¹ | 100 |
| Neisseria gonorrhoeae | purified gDNA | 1 × 10⁹ copies × mL⁻¹ | 100 |
| Neisseria meningitidis | purified gDNA | 1 × 10⁹ copies × mL⁻¹ | 0 |
| Neisseria lactamica | purified gDNA | 1 × 10⁹ copies × mL⁻¹ | 0 |
| Neisseria sicca | purified gDNA | 1 × 10⁸ copies × mL⁻¹ | 0 |
| Neisseria sicca | purified gDNA | 1 × 10⁹ copies × mL⁻¹ | 50 |
| Neisseria sicca* | extracted from cells | 1 × 10⁷ CFU × mL⁻¹ | 75 |
| Chlamydia trachomatis | extracted from cells | 1 × 10⁴ CFU × mL⁻¹ | 0 |
| Escherichia coli* | extracted from cells | 1 × 10⁷ CFU × mL⁻¹ | 0 |
| Proteus mirabilis* | extracted from cells | 1 × 10⁷ CFU × mL⁻¹ | 0 |
| Candida albicans* | extracted from cells | 3.5 × 10⁵ CFU × mL⁻¹ | 0 |
| Staphylococcus aureus* | extracted from cells | 5.3 × 10⁶ CFU × mL⁻¹ | 0 |
| NTC | Nucleic acid free | Nuclease free H₂O | 0 |

For this assay, cross-reactive amplification was observed with N. sicca and N. lactamica nucleic acid material (Table 11). For N. sicca, amplification only occurred at concentrations above the FDA medically relevant recommendation of 1×10⁶ CFU×mL⁻¹ (U.S. Department of Health and Human Services, Food and Drug Administrations, 2011, Draft Guidance for Industry and Food and Drug Administration Staff; Establishing the Performance Characteristics of In Vitro Diagnostic Devices for Chlamydia trachomatis and/or Neisseria gonorrhoeae: Screening and Diagnostic Testing). In addition, even at the highest concentrations evaluated, N. sicca amplification was significantly delayed (≥16 minutes) relative to the average Tp for the same concentration of N. gonorrhoeae at times well beyond the assay cutoff N. lactamica nucleic acid material amplification, in addition to a significant delay relative to N. gonorrhoeae, resulted in curves with a shallow slope and a significantly reduced maximal fluorescence relative to N. gonorrhoeae reactions. Using the associated Roche or Bio-Rad real-time cycler analysis packages (vide supra) for calling reactions as positive or negative, all other organisms tested resulted in negative calls.

### Example 9: Sensitivity Testing

A negative urine matrix was spiked with titred C. trachomatis at various concentrations (10⁴ IFU/mL to 1 IFU/mL). Bacterial stock was serially diluted in PBS before addition to the urine matrix at the desired concentration Extracted samples were amplified using LAMP primers and a molecular beacon probe as indicated. Reaction conditions were equivalent to those described above in Example 3. Amplification signal was obtained with concentrations as low as 0.05 IFU/reaction (see Table 12). Results are classified by the time to positive: A having Tp in less or equal to 10 minutes, B having Tp between 10 minutes and 15 minutes (inclusive), C having Tp greater that 15 minutes. NT indicates that this combination was not tested.

TABLE 12
Sensitivity Testing with Different Primer Sets and Corresponding Beacons

| | | Primer | Beacon | 10³ | 100 | 10 | 4 | 2 |
|---|---|---|---|---|---|---|---|---|
| Organism | Target | Set # | # | IFU/mL | IFU/mL | IFU/mL | IFU/mL | IFU/mL |
| CT | 23S | Set-1 | MB1 | A | A | A | B | B |
| CT | 23S | Set-1 | MB2 | NT | NT | A | B | B |
| CT | 23S | Set-3 | MB1 | A | A | B | NT | C |
| CT | 16S | Set-49 | MB23 | B | NT | NT | C | C |

Sensitivity of a variety of assays were also evaluated (Table 13, indicated CFU is per 50 μl extraction, 5 μl of which was used per reaction). Dilutions of titred N. gonorrhoeae stocks were prepared in PBS (1× diluted from 10×, Ambion CN# AM9624 in nuclease free water, Ambion, CN# AM9932) and spiked into neat urine samples followed by extraction using standard methods. Five μL of nucleic acid from the indicated total CFU per extraction served as template for assay RTLAMP reactions. As indicated in Table 13, most assays combined with Molecular Beacons for detection were sensitive to at least 5 CFU/extraction. Results are classified by the time to positive (Tp) from reaction initiation as follows: “A” indicates a Tp of less than or equal to 9 minutes, “B” indicates a Tp of between 9 minutes and 15 minutes (inclusive), “C” indicates a Tp of greater than 15 minutes and “n.d.” indicates that the assay was not performed.

TABLE 13
Assay Sensitivity

| | | Primer | Beacon | Average Tp | | | | | |
|---|---|---|---|---|---|---|---|---|---|
| Organism | Target | Set # | # | 0.5 CFU | 1 CFU | 5 CFU | 50 CFU | 500 CFU | NTC |
| NG | rsmB | Set-80 | MB34 | B | A | A | A | A | C |
| NG | rsmB | Set-86 | MB34 | n.d. | n.d. | C | n.d. | C | C |
| NG | rsmB | Set-87 | MB34 | n.d. | n.d. | C | n.d. | C | C |
| NG | rsmB | Set-88 | MB34 | n.d. | n.d. | C | n.d. | B | C |
| NG | rsmB | Set-89 | MB35 | n.d. | n.d. | B | n.d. | n.d. | C |
| NG | 23S | Set-90 | MB27 | n.d. | A | A | A | A | C |
| NG | 23S | Set-64 | MB26 | B | B | B | B | B | C |

For swab infused samples, an initial bench protocol was tested which included direct emersion of the swab into undiluted lysis buffer. Detection was very limited for the 1 CFU per extraction concentration (20%) and even more limited for the 0.5 CFU per extraction concentration (data not shown). The swab bench protocol was then adjusted to more closely mimic the urine extraction, specifically by including the same dilution of the lysis buffer with PBS as would result from addition of a urine specimen. This resulted in a 78% improvement, from 20% to 98%, in the frequency of 1 CFU extraction sample detection.

### Example 10: Limit of Detection Estimation

A negative urine matrix was spiked with titred C. trachomatis at various concentrations (10 IFU/mL, 4 IFU/mL, and 2 IFU/mL). Similarly swabs (BD BBL culture Swab EZ Collection and Transport System single swab Fisher Cat# 220144) were infused with C. trachomatis diluted to the same concentrations as used in the urine. Bacterial stock was serially diluted in PBS before addition to the urine matrix or infused to the swab at the desired concentration. For each experiment (for each bacterial serial dilution), one nucleic acid extraction was performed from CT in urine or on a swab at 10 IFU/mL, 10 extractions from samples at 4 IFU/mL, 10 extractions from samples at 2 IFU/mL and one extraction from negative urine or swab matrix. The experiment was repeated 3 times on different days by different operators. One tenth of each extracted sample was amplified using the LAMP primers (Set-1) and the molecular beacon probe MB2 listed in Table 7. In this example the 25 ul reaction contained the Isothermal buffer 1× (New England Biolabs) supplemented with 6.8 mM MgCl₂, 1.6 mM dNTP, 200 nM of molecular beacon (Sigma Aldrich), primers (2 μM of F3 and B3; 0.2 μM of FIP and BIP; 8 μM of LF and LB), 12 Units of Bst2 polymerase (New England Biolabs), 7.5 Units RTx Warmstart (New England Biolabs), and nucleic acid template or water (as no template control). The reactions were incubated at 63° C. and kinetics were monitored using the Roche real-time Lightcycler96 (Roche). Two RT-LAMP reactions were run per extraction. Reactions were scored positive if their Cq were below 15 cycles. The frequency detection of CT in urine or swab was calculated based on the number of positive reactions divided by the total number of reactions (Table 14). All reactions originating from samples at 10 IFU/mL were positives, those originating from negative swab or urine samples were negative. The limit of detection for this assay is estimated to be around 4 IFU/mL for both urine and swab samples. Bacterial load is the concentration in the starting material (urine or swab) 0.5 mL is used for the extractions. Detection was determined to be positive if Tp was less than 15 minutes.

TABLE 14
Limit of Detection

| | Specimen | Bacterial | Number of wells | % |
|---|---|---|---|---|
| Organism | Type | Load | detected | detected |
| CT | Urine | 4 IFU/mL | 64/68 | 94.1 |
| CT | | 2 IFU/mL | 35/60 | 58.3 |
| CT | Swab | 4 IFU/mL | 60/64 | 93.7 |
| CT | | 2 IFU/mL | 39/60 | 65 |

### Example 11: Limited Primer Sets

To assess the contribution of each primer set to the RTLAMP reaction, we also investigated use of just the inner primers or the inner primers plus the loop primers and compared those reactions to the complete 6 primer RTLAMP reaction, using a Molecular Beacon for detection, for both CT and NG targets. Table 15 provides an example using an assay comprised of Set-1(Ps6) and MB1 (specific for CT 23S) at different target concentrations. Interestingly and noteworthy, the reaction still proceeds when the F3/B3 primers (Set-28) are excluded. The absence of F3/B3 appears to have an impact on sensitivity, specifically consistency at low concentrations (Table 15, indicated IFU is per mL of sample, 0.5 mL are used for the extraction, 5 uL of which was used per RTLAMP reaction). The reaction does proceed if only the inner primers are included (Set-41) with substantial delays in the onset of reaction at the highest concentration tested and the sensitivity being poor. Results are classified by the time to positive: A having Tp in less or equal to 10 minutes, B having Tp between 10 minutes and 15 minutes (inclusive), C having Tp greater that 15 minutes. ND indicates that no amplification was detected.

TABLE 15
Contribution of Primer Pairs

| | Primer | Assay Tp | | | | |
|---|---|---|---|---|---|---|
| Organism | Set # | 500 IFU | 10 IFU/mL | 2 IFU/mL | | NTC |
| CT | Set-1 | A | B | B | B | ND |
| CT | Set-28 | A | B | B | ND | ND |
| CT | Set-41 | C | ND | ND | ND | ND |

The assay was repeated for NG targets using Set-80 (Psl) and MB34, specific for NG rsmB. Interestingly and noteworthy, the reaction still proceeds when the F3/B3 primers (set-103) are excluded. The absence of F3/B3 appears to have an impact on sensitivity, specifically consistency at low concentrations (Table 16, indicated CFU is per extraction, 5 uL of which was used per RTLAMP reaction). The reaction does not proceed if only the inner primers are included (Set-114). Results are classified by the time to positive (Tp) from reaction initiation as follows: “A” indicates a Tp of less than or equal to 9 minutes, “B” indicates a Tp of between 9 minutes and 15 minutes (inclusive), and “C” indicates a Tp of greater than 15 minutes or no amplification detected (No Call).

TABLE 16
Contribution of Primer Pairs

| | Primer | Assay Tp | | | | |
|---|---|---|---|---|---|---|
| Organism | Set # | 500 CFU | 5 CFU | 1 CFU | | NTC |
| NG | Set-80 | A | B | B | B | C |
| NG | Set-103 | A | B | C | B | C |
| NG | Set-114 | C | C | C | C | C |

### Other Embodiments

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A composition comprising a LAMP primer set and an oligonucleotide probe comprising a detectable label,
wherein said LAMP primer set, when used in a LAMP amplification reaction in the presence of a target nucleic acid, generates an amplicon comprising a first region or a second region, wherein the first region comprises a B1 region and an F1c region and extends from the 5′ end of the B1 region to the 3′ end of the F1c region, and wherein the second region comprises an F1 region and a B1c region and extends from the 5′ end of the F1 region to the 3′ end of the B1c region, and wherein the amplicon further comprises a probe target sequence; and
wherein the oligonucleotide probe binds specifically to said amplicon at the probe target sequence, wherein the probe target sequence overlaps with the first region or the second region.

2. The composition of claim 1, wherein the target nucleic acid comprises a B2 and a B1 region in this order from a 5′ terminal side, and an F2c and an F1c region in this order from a 3′ terminal side.

3. The composition of claim 2, wherein the LAMP primer set comprises:
a forward inner primer comprising an F1c region and an F2 region, wherein said F1c region of the forward inner primer comprises a sequence substantially identical to the F1c region of said target nucleic acid and wherein said F2 region of the forward inner primer comprises a sequence substantially complementary to the F2c region of the target nucleic acid; and
a backward inner primer comprising a B1c region and a B2 region, wherein said B1c region of the backward inner primer comprises a sequence substantially complementary to a B1 region of the target nucleic acid and wherein said B2 region of the backward inner primer comprises a sequence substantially identical to the B2 region of the target nucleic acid sequence.

4. The composition of claim 3, wherein said target nucleic acid further comprises an F3c region 3′ of said F2c region and a B3 region 5′ of said B2 region, and wherein said LAMP primer set further comprises a forward outer primer and a backward outer primer, wherein said forward outer primer comprises a sequence substantially complementary to the F3c region of the target nucleic acid and wherein said backward outer primer comprises a sequence substantially identical to the B3 region of the target nucleic acid.

5. The composition of claim 3, wherein said LAMP primer set further comprises a loop forward primer and a loop backward primer, wherein said loop forward primer comprises a sequence substantially identical to a sequence between said F1c and said F2c region of said target nucleic acid, and wherein said loop backward primer comprises a sequence substantially complementary to a sequence between said B1 and said B2 region of said target nucleic acid.

6. The composition of claim 1, wherein the oligonucleotide probe comprises a sequence substantially complementary to the probe target sequence.

7. The composition of claim 1, wherein the probe target sequence overlaps the first region or the second region of the amplicon by at least 3 nucleotides.

8. The composition of claim 7, wherein the probe target sequence overlaps the first region or the second region of the amplicon by at least 7 nucleotides.

9. The composition of claim 8, wherein the probe target sequence overlaps the first region or the second region of the amplicon by at least 10 nucleotides.

10. The composition of claim 9, wherein the probe target sequence is located completely within the first region or the second region of the amplicon.

11. The composition of claim 1, wherein the probe target sequence overlaps with at least 3 nucleotides, at least 7 nucleotides, at least 10 nucleotides, or all of the F1 region, the F1c region, the B1 region, or the B1c region of the amplicon.

12. (canceled)

13. (canceled)

14. The composition of claim 1, wherein said detectable label is a fluorophore.

15. The composition of claim 1, wherein said oligonucleotide probe further comprises a quencher.

16. The composition of claim 15, wherein said quencher is covalently bound to a terminus of the oligonucleotide probe.

17.-20. (canceled)

21. A method of detecting the presence or absence of a target nucleic acid in a test sample, the method comprising:
mixing the test sample with a reaction mixture comprising a strand displacement DNA polymerase and a LAMP primer set;
exposing said test sample to loop-mediated amplification reaction conditions to generate an amplicon from the target nucleic acid, if present in said test sample, wherein the amplicon comprises a probe target sequence;
contacting the test sample with an oligonucleotide probe comprising a detectable label, wherein the oligonucleotide probe binds specifically to the amplicon at the probe target sequence, if present, wherein the probe target sequence overlaps with a first region or a second region, wherein the first region comprises a B1 region and an F1c region and extends from the 5′ end of the B1 region to the 3′ end of the F1c region, and wherein the second region comprises an F1 region and a B1c region and extends from the 5′ end of the F1 region to the 3′ end of the B1c region; and
detecting the presence or absence of a signal from the detectable label, wherein the presence of said signal is indicative of the presence of the target nucleic acid in the test sample.

22. The method of claim 21, wherein said loop-mediated amplification reaction is performed at a temperature of between about 60° C. and about 67° C.

23. The method of claim 22, wherein said loop-mediated amplification reaction is performed for less than 30 minutes.

24. The method of claim 23, wherein said loop-mediated amplification reaction is performed for less than 15 minutes.

25. (canceled)

26. (canceled)

27. The method claim 21, wherein said reaction mixture further comprises a reverse transcriptase.

28. A method of detecting the presence or absence of a target nucleic acid in a test sample, the method comprising:
providing a test sample suspected of comprising a target nucleic acid, wherein said test sample comprises the composition of claim 1 and a strand displacement DNA polymerase;
exposing said test sample to conditions sufficient to generate an amplicon from the target nucleic acid, if present in said test sample, via a loop-mediated amplification reaction; and
detecting the presence or absence of a signal from the detectable label, wherein the presence of said signal is indicative of the presence of the target nucleic acid in the test sample.

29. (canceled)
